(19) United States
(12) Patent Application Publication
(10) Pub. No.: US 2020/0352991 A1
(43) Pub. Date: Nov. 12, 2020

(54) POLYMER FILMS WITH ANTIMICROBIAL AGENTS

(71) Applicant: The Trustees of Columbia University in the City of New York, New York, NY (US)

(72) Inventors: Shanta M. Modak, River Edge, NJ (US); Chathuranga C. De Silva, Cliffside Park, NJ (US); Anand Arvind Zanwar, Jalna (IN); Mahabaleshwar Hegde, New York, NY (US); Arnab Kumar Ghosh, West Bengal (IN)

(21) Appl. No.: 16/630,779

(22) PCT Filed: Jul. 27, 2018
(86) PCT No.: PCT/US18/44247

§ 371 Date: Jan. 13, 2020

Related U.S. Application Data
(60) Provisional application No. 62/537,790, filed on Jul. 27, 2017.

(51) Int. Cl. A61K 33/38; A61K 47/34; A61K 47/36; A61K 47/38; A61K 47/44; A61K 31/505; A61K 31/7036; A61K 36/886; A61K 33/30; A61K 36/28; A61K 36/537; A61K 47/10; A61L 29/08; A61K 36/55; A61K 33/18; A61K 31/4174; A61K 31/201; A61K 31/155; A61K 36/185; A61K 31/121; A61K 31/015; A61K 36/752; A61K 36/53; A61K 36/82; A61K 31/355; A61K 31/375; A61K 47/14; A61K 47/20; A61P 31/04; A61K 47/22; A61K 47/12; A61K 47/32; A61K 9/06; A61K 36/45; A61K 31/345

(52) U.S. Cl. CPC A61K 33/38; A61L 2300/204; A61K 47/36; A61K 47/38; A61K 47/44; A61K 31/505; A61K 31/7036; A61K 36/886; A61K 33/30; A61K 36/28; A61K 36/537; A61K 47/10; A61L 29/085; A61K 36/55; A61K 33/18; A61K 31/4174; A61K 31/201; A61K 31/155; A61K 36/185; A61K 31/121; A61K 31/015; A61K 36/752; A61K 36/53; A61K 36/82; A61K 31/355; A61K 31/375; A61K 47/14; A61K 47/20; A61P 31/04; A61K 47/22; A61K 47/12; A61K 47/32; A61K 9/06; A61K 36/45; A61K 31/345; A61L 2300/404; A61L 2420/02; A61L 2300/104; A61L 2300/30; A61L 2300/206; A61K 47/34

(57) ABSTRACT

The present technology relates to film forming compositions comprising antimicrobial agents, as well as to methods of inhibiting bacterial growth, controlling the rate of release of an antimicrobial agent from a film forming polymer, coating a medical device, rendering the inner lumen of a medical device biofilm resistant, and treating a human or animal.

### CROSS REFERENCE TO RELATED APPLICATIONS

This application is a National Stage Application of, and claims priority to International Application Serial No. PCT/US2018/044247 filed on Jul. 27, 2018, which claims priority to U.S. Provisional Application No. 62/537,790 filed Jul. 27, 2017, the disclosures of which are hereby incorporated by reference in their entirety.

### BACKGROUND

The present technology relates to antimicrobial compositions that are useful for a variety of applications, including the treatment of wounds and the coating of medical devices.

Surface wounds can vary in size, shape, etiology, and thus remain a challenge to treat. Proper wound care of surface wounds is challenging given the different causes and anatomic locations of wounds. Chronic non-healing wounds are a significant source of morbidity and mortality in diabetic patients. The emotional and economical toll of chronic diabetic wounds is enormous.

Novel molecular strategies to treat these wounds are based on promotion of angiogenesis, reduction of inflammation and prevention of wound infection. Currently marketed products generally include topical creams or ointments that are applied to surface wounds; however, these have disadvantages. When creams or ointments are applied topically to a wound, they tend to migrate from the wound site, flow or rub off unless a protective dressing is applied to keep them in place and in contact with the wound. However, dressings can be difficult to apply onto, or keep attached to, certain locations of the body; they also need to be changed frequently until a wound is healed. Moreover, occlusion of a wound caused by a dressing can increase the possibility of infection, since the occlusion dressing provides a warm and moist environment, optimal for bacterial multiplication.

Film forming products containing antimicrobial agents such as silver or antibiotics are available commercially. These can be used without the need for a wound dressing. However, none of these compositions provide sustained, broad spectrum antimicrobial efficiency.

There is an enhanced effort to develop drugs that accelerate wound healing. Several plants and herbs have been used experimentally to treat skin disorders including wound healing in traditional medicine. Several of these wound healing agents are explored in the present formulations. Flaxseed is one of the oldest cultivated plants in the world and is cultivated for its fiber and oil. Flaxseed oil and its derivatives are rich source of the essential fatty acid, alpha-linolenic acid, which is a biological precursor to omega-3 fatty acids. Several animal studies suggested that omega-3 fatty acids of this plant may have anti-inflammatory as well as wound healing properties.

The effects of topical application of linseed oil (flaxseed oil) topical on burn wounds healing in rat model have herein been investigated. Zinc salts and calendula oil have also been shown to enhance wound healing. Silver sulfadiazine is the most commonly used topical antimicrobial agent for controlling wound infections, especially in burns. A combination of wound healing agents and antimicrobial agents can act synergistically to promote wound healing and control infection. Therefore, combinations of one or more of flaxseed oil, zinc salt, calendula oil or silver sulfadiazine can prove an effective treatment modality for debilitating burn and chronic diabetic wounds.

A need exists for film forming products that can heal a wound, stay in place and provide sustained and predictable treatment, all while minimizing the chances of infection and avoiding the need for constant changing of dressings or reapplication of ointment. Compositions that contain botanicals as antimicrobials are also desirable.

### SUMMARY OF THE DISCLOSED TECHNOLOGY

In certain embodiments, the present technology is directed to compositions comprising a film-forming polymer and an antimicrobial. In certain embodiments, the present technology is directed to a film forming composition comprising: one or more film-forming polymer and an antimicrobial agent, wherein the film forming composition provides controlled release of the antimicrobial agent onto a surface when the film forming composition is contacted with the surface.

In certain embodiments, the present technology is directed to a film forming composition comprising: a film forming polymer; a botanical; and an antimicrobial agent;

wherein the antimicrobial agent is: a botanical; a silver salt; a zinc salt; polymyxin; chlorhexidine or its salts; benzalkonium chloride; bacitracin; neomycin; clindamycin; polymyxin; bactroban; povidone iodine; gentamicin; gentian violet; mupirocin; dicloxacillin; undecylinic acid; nitrofurazone; miconazole; a cephalosporin; cranberry seed oil; N-acetyl cysteine; berberin; copper sulfate or a combination thereof;

wherein the film forming composition provides controlled release of the antimicrobial agent onto a surface when the film forming composition is contacted with the surface.

In certain embodiments, the present technology is directed a film forming composition comprising: a film-forming polymer and an antimicrobial agent, wherein the film forming composition provides controlled release of the antimicrobial agent onto a surface when the film forming composition is contacted with the surface.

In certain embodiments, the present technology is directed to a film forming composition comprising a mixture of: (a) a pH-degradable polyacetal co-polymer or polyacetal-octanediol conjugate, or polyketal co-polymer or polyketal-octanediol conjugate, or other suitable polyacetal or polyketal conjugate; (b) a hydrophilic polymer; and (c) a hydrophobic-hydrophilic polymer.

In certain embodiments, the present technology is directed to a chlorhexidine-free coating composition that increases the infection resistance of a medical device when coated on the medical device, the coating composition comprising: a triple film forming polymer coating composition (FTP) comprising polyacetal-octanediol conjugate (PA-OCT or PA-OCT-80); a first polyurethane composition; a second polyurethane composition; a silicone adhesive; decanediol; and a solvent wherein the solvent is methanol, ethanol or tetrahydrofuran.

In certain embodiments, a composition herein increases the infection resistance of a medical device by 1,000 to 10,000 fold when coated on the medical device.

In certain embodiments, the present technology is directed to methods of treating wounds, inhibiting microbial growth, controlling the rate of release of an antimicrobial agent from a film forming polymer, coating a medical device, and rendering the inner or outer lumen (also referred to herein as “inner surface” or “outer surface”) of a medical device biofilm resistant; as well as medical devices coated with the compositions herein.

In certain embodiments, the present technology is directed to a coating composition that increases the infection resistance of a medical device when coated on the medical device, the coating composition comprising:
(a) 1 to 5% chlorhexidine;
(b) 0.1 to 1% of a zinc salt;
(c) 0.2 to 5% of a triple film forming polymer coating composition (FTP) comprising polyacetal-octanediol conjugate (PA-OCT or PA-OCT-80);
(d) 0.2 to 5% of a first polyurethane composition;
(e) 0.2 to 5% of a second polyurethane composition;
(f) 0.2 to 5% of a silicone adhesive;
(g) 0.5 to 3% decanediol; and
(h) a solvent, wherein the solvent is methanol, ethanol or tetrahydrofuran.

In certain embodiments, the present technology is directed to a method of rendering the inner lumen of a medical device biofilm resistant, the method comprising; contacting the inner lumen with a composition herein. In certain embodiments, the inner surface of the medical device is contacted with the composition for 5 to 60 seconds, and then removed from contact and dried for 24 to 48 hours. In certain embodiments, the biofilm resistance of the inner lumen of the medical device is 1,000 to 10,000 fold more than the biofilm resistance of the inner lumen of a medical device that has not been contacted with the composition.

In certain embodiments, the present technology is directed to use of a composition of claim 1 for treatment of a human or animal.

### BRIEF DESCRIPTION OF THE DRAWINGS

FIGS. 1A and 1B show the zones of inhibition of various compositions herein, compared with those of commercial products.

FIGS. 2A, 2B and 2C show duration of activity of various compositions herein on various microorganisms, compared with those of commercial products.

FIGS. 3A-3D show, duration of activity of various compositions herein on various microorganisms, compared with those of commercial products.

FIG. 4A shows the results of a film retention time study of various compositions at different rinsing temperatures.

FIG. 4B shows retention of antimicrobial efficacy after rinsing at 25° C. for a) 10% FTP-A, b) 15% FTP-A and c) Cream-A. The efficacy was measured using an ex-vivo pig-skin model. Test organism was S. aureus.

FIG. 5 shows the effect of polyacetal polymer concentration in a film forming triple polymer on the release of antimicrobial agent characterized by zone of inhibition, at various concentrations of polymer.

FIG. 6 shows quantitative bacterial adherence of compositions herein and commercially available urinary catheters.

FIG. 7 shows qualitative bacterial adherence of compositions herein and commercially available urinary catheters.

FIGS. 8A and 8B show results of Ex vivo pigskin rapid kill after 2 hours for S. aureus.

FIGS. 9A and 9B show results of Ex vivo pigskin rapid kill after 2 hours for P. aeruginosa.

FIGS. 10A and 10B show results of testing on adherence for catheters coated with compositions in accordance with certain embodiments herein.

### DETAILED DESCRIPTION

Unless otherwise indicated, all percentages discussed herein refer to weight percent.

As used herein, the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “alkyl” denotes a branched, unbranched, or cyclic saturated hydrocarbon having from one to the number of carbon atoms designated (e.g., C₁-C₁₀ alkyl). Examples of alkyl include, but are not limited to, methyl, ethyl, n-propyl, isopropyl, n-butyl, t-butyl, n-hexyl, n-octyl, and the like. It will also be appreciated that the prefix “n” denotes an unbranched, acyclic group. For example, “C3 n-alkyl” denotes an unbranched propyl group, which can also be referred to as “n-propyl”. For a diol comprising a Cn alkyl group, the Cn alkyl group can be arranged in any number of ways known to a person of ordinary skill in the art (e.g., branched, unbranched, cyclic).

As used herein, the term “diol” denotes a compound that comprises at least two hydroxyl groups. Representative diols include, but are not limited to, therapeutic agents that comprise at least two hydroxyl groups. A therapeutic agent that contains a diol comprises at least two hydroxyl groups and a “therapeutic agent core”.

As used herein, the term “therapeutic agent core” denotes a therapeutic agent without (in the absence of) two of the at least two hydroxyl groups in the therapeutic agent.

Therapeutic agents include, but are not limited to, drugs, agricultural agents, proteins, small molecule therapeutics, carbohydrate and peptides.

Agricultural agents include, but are not limited to, pesticides, herbicides, fungicides, insecticides, nematode control agents, antihelmintics, and nutrients.

A drug that contains a diol comprises at least two hydroxyl groups and a “drug core”. As used herein the term “drug core” denotes a drug without (in the absence of) two of the at least two hydroxyl groups in the drug.

As used herein, “surface wound” means any wound to the surface of a patient's body (including but not limited to the skin, nails, scalp, mucosa, any oral surface including tongue, inside of cheek, palate or throat), including but not limited to a burn, ulcer, abrasion, cut, diabetic wound or decubitus ulcers.

As used herein, “antimicrobial” or “antimicrobial agent” means an agent that kills microorganisms or stops their growth. These include, but are not limited to, antibacterial agents, antifungal agents, antiviral agents, microbiocidal agents, antibiotics, bactericidal agents, bacteriostatic agents, disinfectants and antiseptics.

As used herein, “a botanical” means a composition from a plant source, including an essential oil, essential oil ingredient, or botanical extract.

As used herein, “essential oil” (EO) is a volatile oil obtained from a plant or an animal source that comprises one or more active agent (also referred to herein as an Isolated Component or “IC” or “constituent” or “ingredient” or “botanical ingredient” or “essential oil ingredient”) which can be, for example but not by way of limitation, a monoterpene or sesquiterpene hydrocarbon, alcohol, ester, ether, aldehyde, ketone, or oxide. Essential oils are commonly extracted by distillation, expression, extraction, resin tapping, wax embedding or cold pressing. Isolated components generally fall into the following categories: acids, alcohols (e.g., monoterpenols or sesquiterpenols), aldehydes, coumarins, esters, ketones, lactones, terpenes (e.g., monoterpenes or sesquiterpenes), oxides, or phenols.

As used herein, “botanical extract” means a composition from a plant source (a botanical) that is prepared by soaking the botanical in a solvent (e.g., water or alcohol). A botanical extract refers to the resultant liquid, which contains the essential oil with the solvent. As described in Examples and data herein, the terminology “(100/oil)” denotes 100% extract or oil.

As used herein, “medical device” means any instrument, apparatus or other article that can be inserted into, or otherwise contacted with, the body of a patient, for diagnosis, treatment, prevention or monitoring of a disease, injury or medical condition.

In certain embodiments, the present technology is directed to methods of treating minor wounds and controlling infection for shorter period of time using a film forming gel comprising two gelling agents, wherein the film forming gel contains one or more wound healing agents or antibacterial agents, and releases the wound healing agents or antibacterial agents within a short period of time.

In certain embodiments, the technology is directed to a film comprising three polymers and a broad-spectrum antimicrobial to treat surface wounds. By incorporating antimicrobial agents, wound-healing agents, and emollients into a film-forming triple polymer (FTP) composition, this technology provides multiple days of antimicrobial activity without the need for daily dressing changes.

In certain embodiments, the FTP described herein can be incorporated into bandages. In particular, broad-spectrum antimicrobial activity has been found to be sustained for 4 days or more.

In certain embodiments, the technology is also directed to a film forming composition with one or more antimicrobial agents suitable for coating medical devices, such that the composition increases the infection resistance of a medical device when coated on the medical device. In further embodiments, the compositions according to the technology herein can reduce infection in surgery, or allow for sustained application of topical treatments for dermatological conditions.

The film forming compositions herein can, in certain embodiments, act like a bandage—that is, they solidify rapidly to protect the surface of the wound, while avoiding the problems associated with occlusion by known bandages. Thus, they can eliminate the need for a separate bandage to cover a wound, and can be the only covering on a wound.

In certain embodiments, the compositions herein rapidly form a film upon application the skin; or can act like a bandage or dressing (thus obviating the need for the additional bandage or dressing) in that they do not rub off.

In certain embodiments, the wound healing agents present in the compositions herein (including the antimicrobial agents) can be released in a controlled manner, thereby prolonging the period in which the treatment is effective, resulting in lower toxicity and a reduction of the negative effect of the antimicrobial agents on the wound's healing process.

Another advantage of the embodiments herein is that the compositions eliminate the need for regular application of topical antimicrobial agents or wound healing agents (such as antibiotic creams and the like), and regular change of dressings. Thus, the compositions herein are easily available for unassisted, self-application by a user. Further, in certain embodiments when the compositions are applied, they create a film in a manner such that they do not wipe off easily. This increases convenience for a user and avoids the necessity of constant reapplication or limit of motion and activity.

Polymers

In certain embodiments, the compositions herein comprise a hydrophilic polymer.

In certain embodiments, the hydrophilic polymer is chitosan or a derivative thereof. Chitosan is a linear polysaccharide derived from the shells of crustaceans, and is composed of randomly distributed β-(1→4)-linked D-glucosamine (deacetylated unit) and N-acetyl-D-glucosamine (acetylated unit). It has been used in connection with bandages for reducing bleeding and has antimicrobial properties. As used herein, a “derivative” of chitosan refers to, for example, chitosan pyrrolidone carboxylic acid, for example, a compound known as Kytamer PCA from Dow Chemical Company.

In certain embodiments, an exemplary composition herein is a film forming triple polymer composition (FTP) that includes a mixture of three polymers. These can be, in various embodiments:

(a) a pH-degradable polyacetal or polyketal co-polymer;

(b) a hydrophilic polymer; and

(c) a hydrophilic-hydrophobic (amphoteric) polymer.

In certain embodiments, the pH-degradable polyacetal co-polymer or polyketal co-polymer is a polyacetal-octanediol conjugate, or a polyketal-octanediol conjugate; or any other alkanediol derivative of polyacetal polymer or polyketal polymer. In various embodiments, any polyacetal or polyketal derivative can be used. Examples include, but are not limited to: polyacetal or polyketal homo- and co-polymers, polyacetal or polyketal main-chain conjugates, polyacetal or polyketal side-chain conjugates, and polyacetal or polyketal block-co-polymers.

In certain embodiments, the hydrophilic polymer is a chitosan-derived hydrophilic polymer. Other useful hydrophilic polymers include hydrophilic polymers such as carboxymethylcellulose (CMC), methylcellulose (MC), hydroxyethylcellulose (HEC), hydroxypropyl cellulose (HPC), ethylcellulose (EC) Hydroxypropyl methyl cellulose, or water-soluble MC and hydroxypropyl MC polymers, derived from pine pulp and commercially known as “Methocel” (available from the Dow Chemical Co., Midland, Mich., USA), nonionic triblock copolymers composed of a central hydrophobic chain of polyoxypropylene (poly(propylene oxide)) flanked by two hydrophilic chains of polyoxyethylene (poly(ethylene oxide)), known as poloxamers; and derivatives or combinations of any of these; in amounts of, e.g., 0.1 to 1% or 0.1 to 5%.

In certain embodiments, the hydrophilic-hydrophobic polymer can be a cellulose ether. The hydrophilic-hydrophobic polymer is a hydrophobically modified hydroxypropylmethylcellulose. One exemplary hydrophobically modified hydroxypropylmethylcellulose is hydroxypropylmethylcellulose stearoxy ether, available under the trade name Sangelose® from Daido Chemical Corporation (Osaka, Japan).

In certain embodiments, the compositions herein comprise a film forming hydrophilic polymer, hydrophilic-hydrophobic polymer (for example, a chitosan or chitosan derivative with a cellulose ether such as, e.g., hydroxypropyl methylcellulose (HPMC), a derivative of cellulose (pulp), modified with a stearyl group (available under the trade name Sangelose® from Daido Chemical Corporation, Osaka, Japan).

In certain embodiments, the compositions for coating medical devices herein comprise a film forming hydrophilic polyurethane polymer of various hardness. These can include, e.g., any medical-grade aliphatic polyether polyurethanes, such as those available under the trade name (Tecofex® polyurethane 93A and 60D) (FDP-M).

In certain embodiments, the compositions for coating medical devices herein comprise film forming hydrophilic polyurethane polymer of various hardness—for example, those available under the trade names Tecoflex®, e.g., polyurethane 93A and 60D from Lubrizol Corporation (Wickliffe, Ohio, USA) and polyacetal-octanediol or polyketal-octanediol polymer (FTP-M).

In certain embodiments, the compositions herein include or are combined with biodegradable or thermoresponsive polymers such as those disclosed in International Patent Application No. PCT/US2015/063669 (published as WO/2016/090103 on Jun. 9, 2016), incorporated herein by reference. These include but are not limited to, in various embodiments, any of the following suitable compositions disclosed in that publication, and listed below.

In certain embodiments, a class of compounds of formula (I):

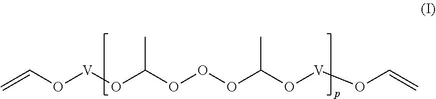

wherein V is

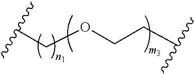

each D may be the same or different and is

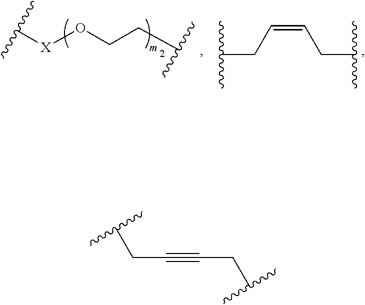

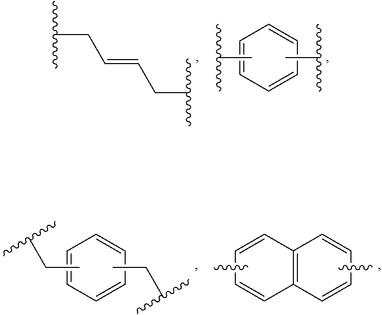

or a therapeutic agent core; each
n₁ may be the same or different and is a integer between 2 and 10;
each m₁ may be the same or different and is an integer between 0 and 20;
each X may be the same or different and is C₂-C₁₀ alkyl;
each m₂ may be the same or different and is an integer between 0 and 20; and
p is an integer between 3 and 200.

In certain embodiments, each D may be the same or different and is

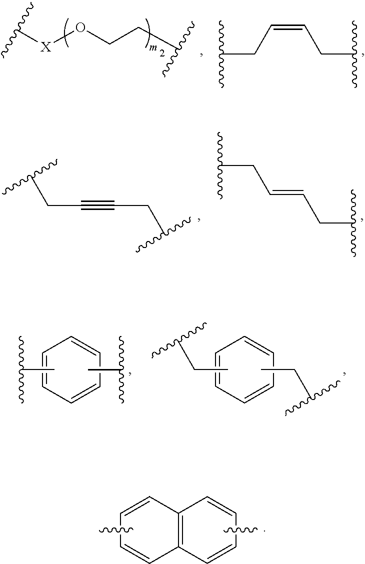

or a therapeutic agent core; and water or a liquid chemical carrier.

In certain embodiments, each D may be the same or different and is

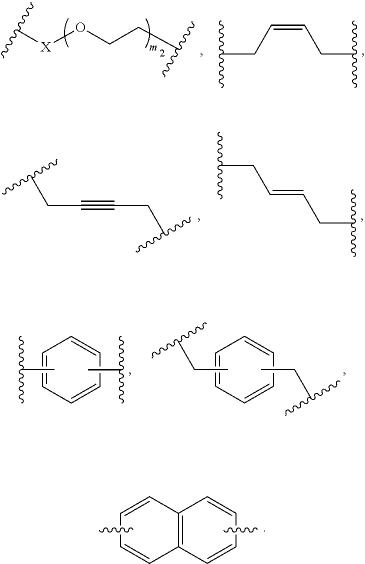

or a therapeutic agent core; or a pharmaceutical composition thereof.

In certain embodiments, a biodegradable gel comprising a compound of formula (I) cross-linked with a linker at a terminus of the compound of formula (I), and wherein the linker is bonded to a plurality of compounds of formula (I).

In certain embodiments, a method of making a gel, comprising cross-linking a compound of formula (I) with a trifunctional linker.

In certain embodiments, a method of delivering a therapeutic agent to a wound comprising, administering a biodegradable gel comprising a compound of formula (I) cross-linked with a linker at a terminus of the compound of formula (I), and wherein the linker is bonded to a plurality of compounds of formula (I); and a therapeutic agent, wherein said gel degrades at pH from about 5 to about 6.5 to release said therapeutic agent.

In certain embodiments, a class of compounds of formula (II):

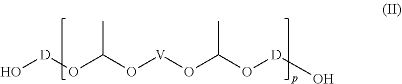

wherein,

### V is

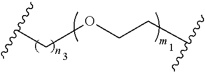

each D may be the same or different and is

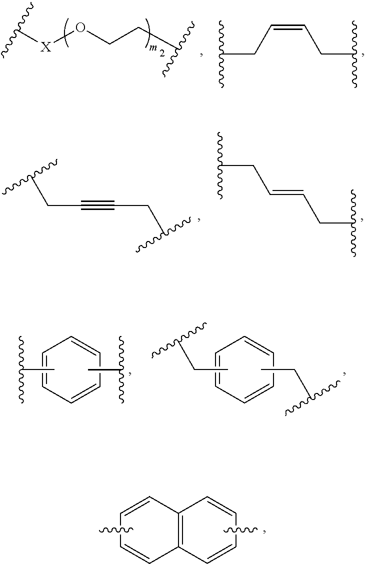

or a therapeutic agent core;
each n₁ may be the same or different and is an integer between 2 and 10;
each m may be the same or different and is an integer between 0 and 20;
each X may be the same or different and is C₂-C₁₀ alkyl;
each m₂ may be the same or different and is an integer between 0 and 20; and
p is an integer between 3 and 200.

In certain embodiments, a biodegradable gel comprising a compound of formula (II) cross-linked with a linker at a terminus of the compound of formula (II), and wherein the linker is bonded to a plurality of compounds of formula (II).

In certain embodiments, a method of making a gel, comprising cross-linking a compound of formula (II) with a trifunctional linker; in some embodiments, the trifunctional linker comprising a triisocyanate.

In certain embodiments, compositions comprising a compound of formula (I) wherein each D may be the same or different and is

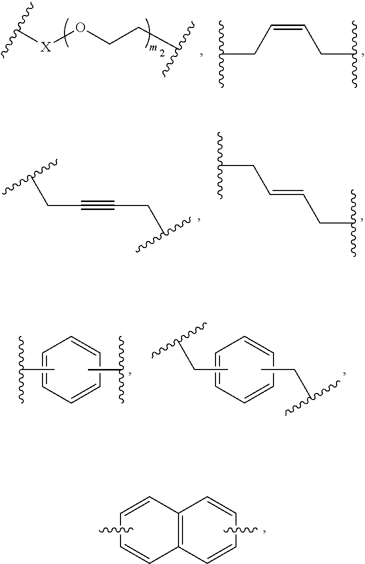

or a therapeutic agent core; and a pharmaceutically acceptable carrier.

In certain embodiments, a method for treating wounds in a subject, the method comprising administering to a subject a therapeutic amount of a compound of formula (I), wherein each D may be the same or different and is

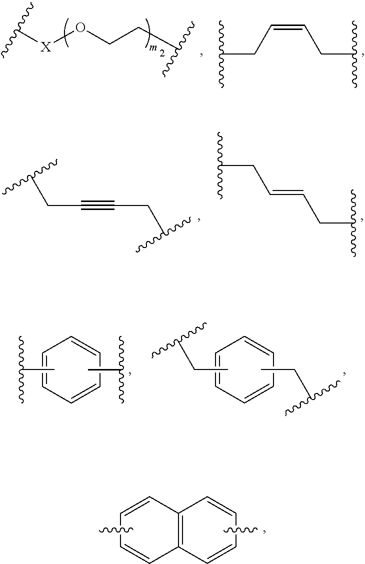

or a therapeutic agent core; or a pharmaceutical composition thereof.

In certain embodiments, compositions comprising a compound of formula (I), wherein each D may be the same or different and is

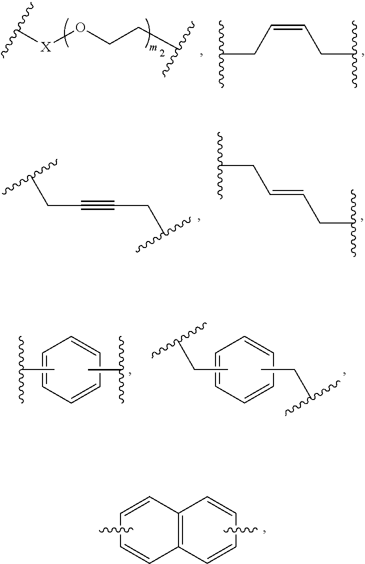

or a therapeutic agent core; and water or a liquid chemical carrier.

In certain embodiments, compositions comprising a compound of formula (II) wherein each D may be the same or different and is

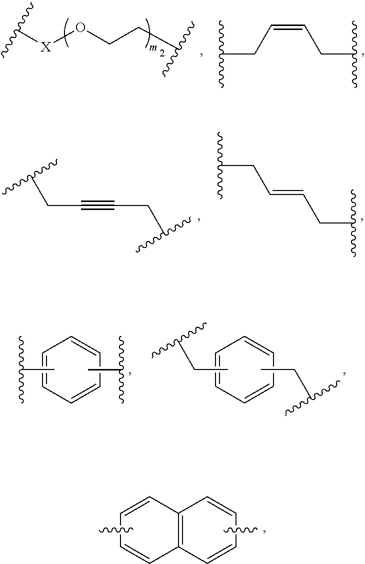

or a therapeutic agent core; and a pharmaceutically acceptable carrier.

In certain embodiments, compositions comprising a compound of formula (II), wherein each D may be the same or different and is

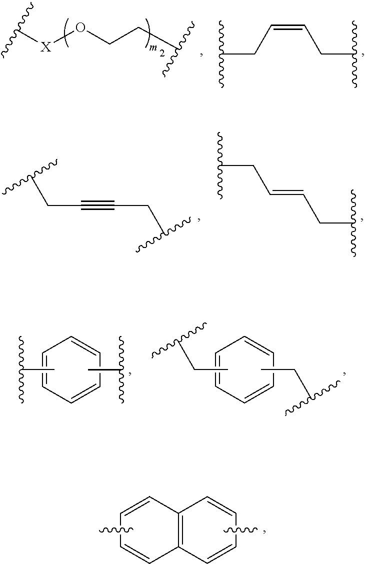

or a therapeutic agent core; and a water or a liquid chemical carrier.

In certain embodiments, a class of compounds of formula (III):

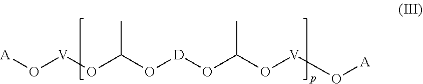

wherein,

### A is

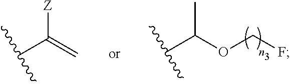

### F is

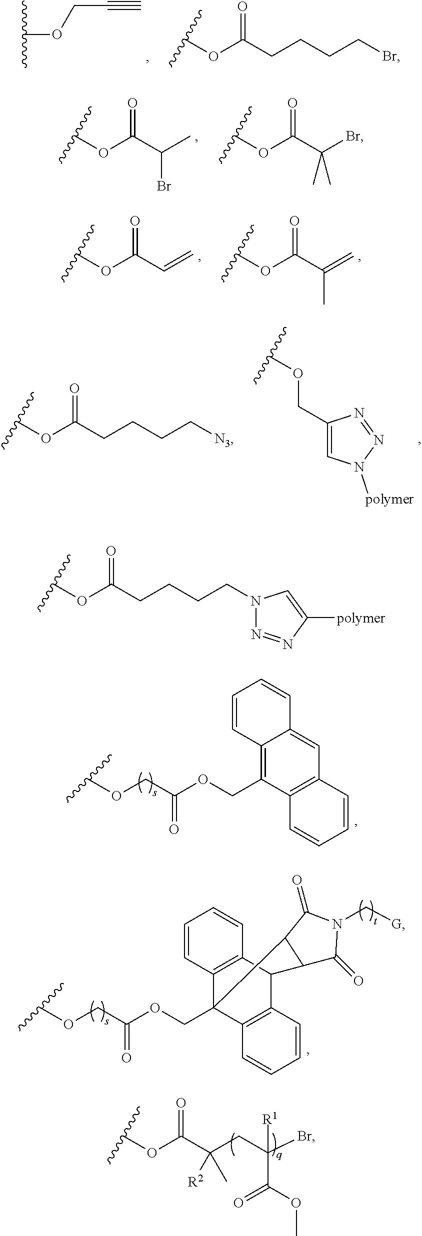

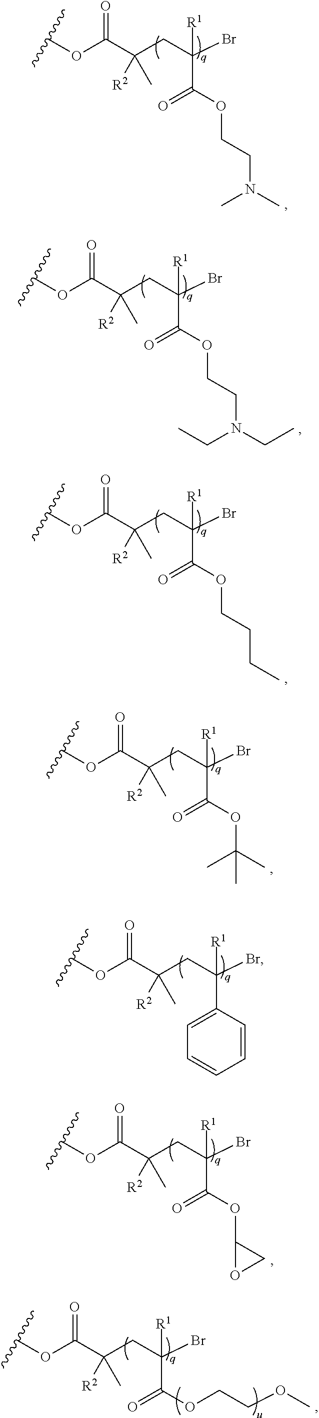

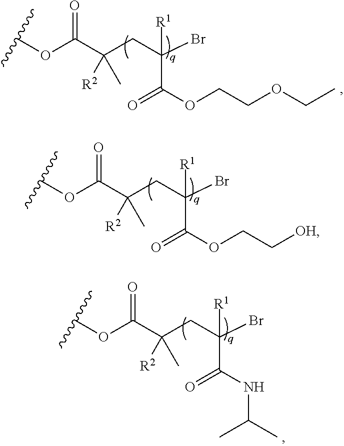

or a polymer;
Z is a polymer, aryl, hetero-aryl, or vinyl;
v is

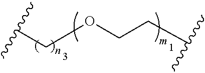

each D may be the same or different and is

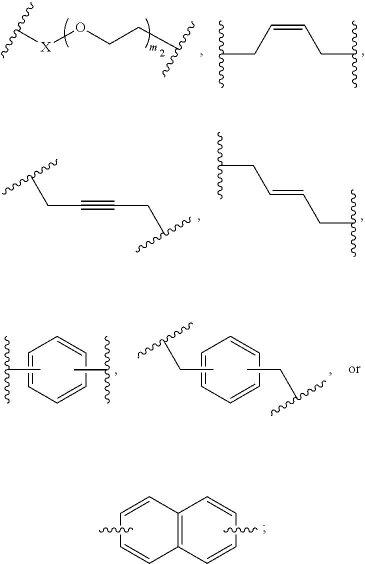

each n₁ may be the same or different and is an integer between 2 and 10; each m₁ may be the same or different and is an integer between 0 and 20;
each X may be the same or different and is C₂-C₁₀ alkyl;
each m₂ may be the same or different and is an integer between 0 and 20;
n₃ is an integer between 2 and 10;
p is an integer between 3 and 200;
q is an integer between 1 and 100;
s is an integer between 1 and 10;
t is an integer between 1 and 10;
u is an integer between 1 and 100;
G is a polymer, aryl, or alkyl;
R¹ is H or CH₃; and
R² is H or CH₃.

In certain embodiments, a class of compounds of formula (III):

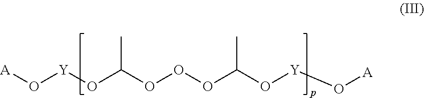

wherein,

### A is

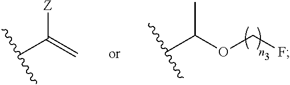

### F is

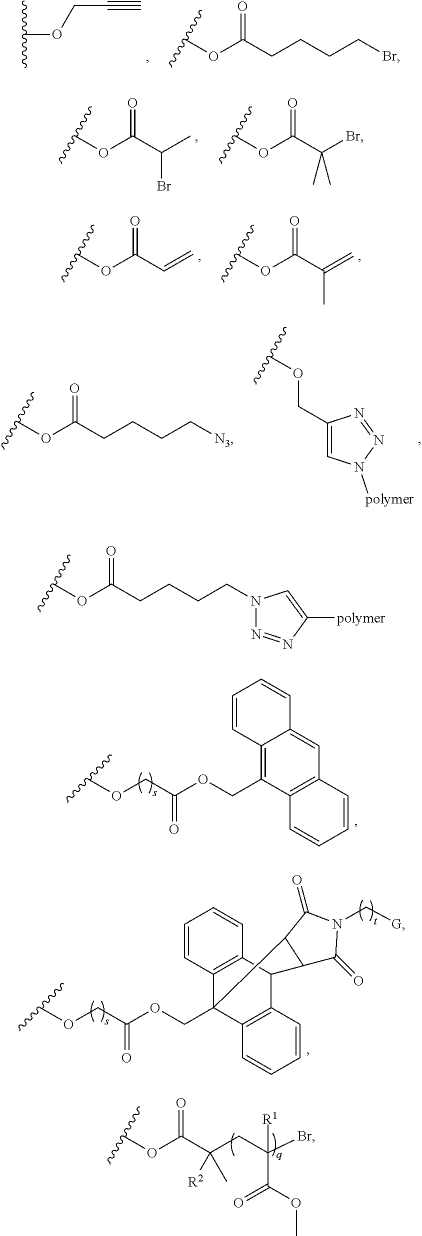

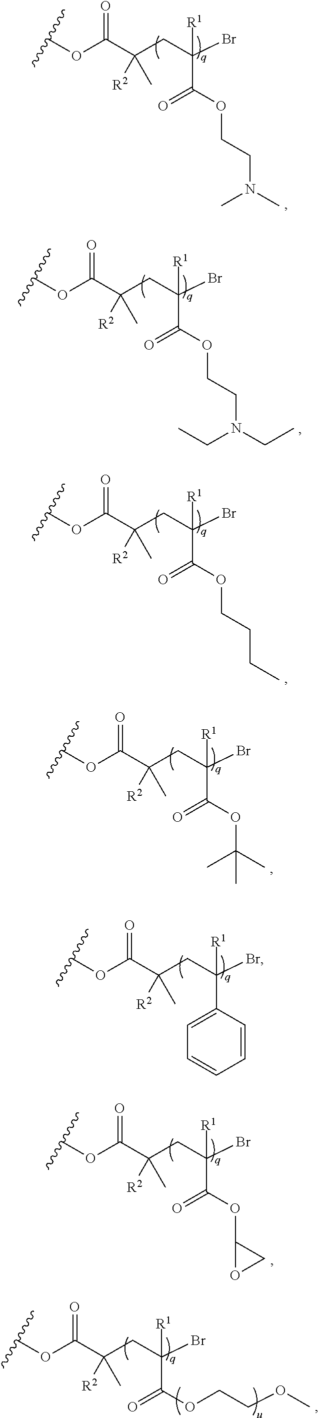

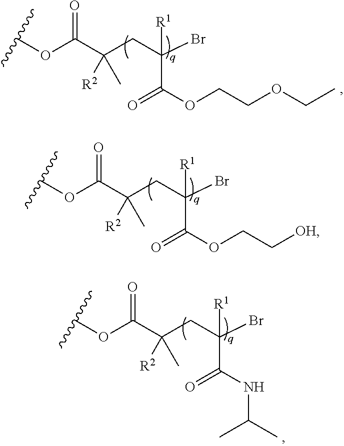

or a polymer;
Z is a polymer, aryl, hetero-aryl, or vinyl;

### V is

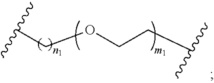

each D may be the same or different and is

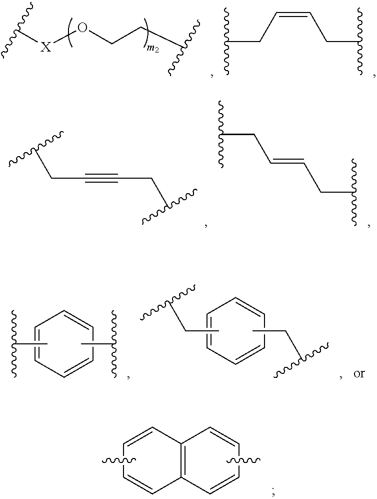

or a therapeutic agent core;
each n₁ may be the same or different and is an integer between 2 and 10;
each m₂ may be the same or different and is an integer between 0 and 20;
each X may be the same or different and is C₂-C₁₀ alkyl;
each m₂ may be the same or different and is an integer between 0 and 20;
n₃ is an integer between 2 and 10;
p is an integer between 3 and 200;
q is an integer between 1 and 100;
s is an integer between 1 and 10;
t is an integer between 1 and 10;
u is an integer between 1 and 100;
G is a polymer, aryl, or alkyl;
R₁ is H or CH₃; and
R₂ is H or CH₃.

In certain embodiments, a biodegradable gel comprising a compound of formula (III) cross-linked with a linker at an alkyne or azide terminus of the compound.

In certain embodiments, a method of making a gel, comprising crosslinking a compound of formula (III) with a trifunctional linker.

In certain embodiments, a class of compounds of formula (IV)

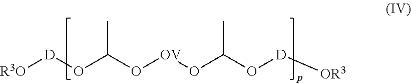

wherein,

### R³ is

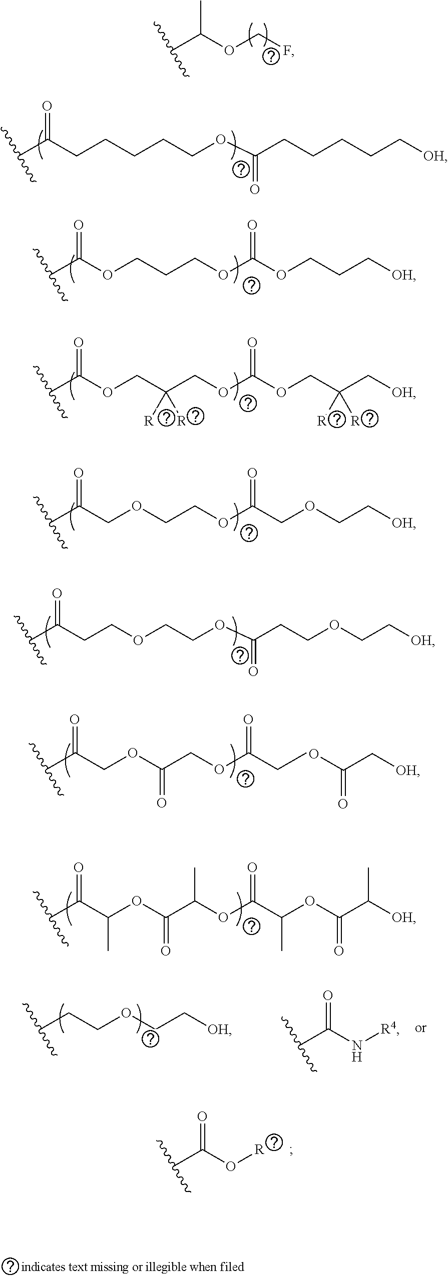

### F is

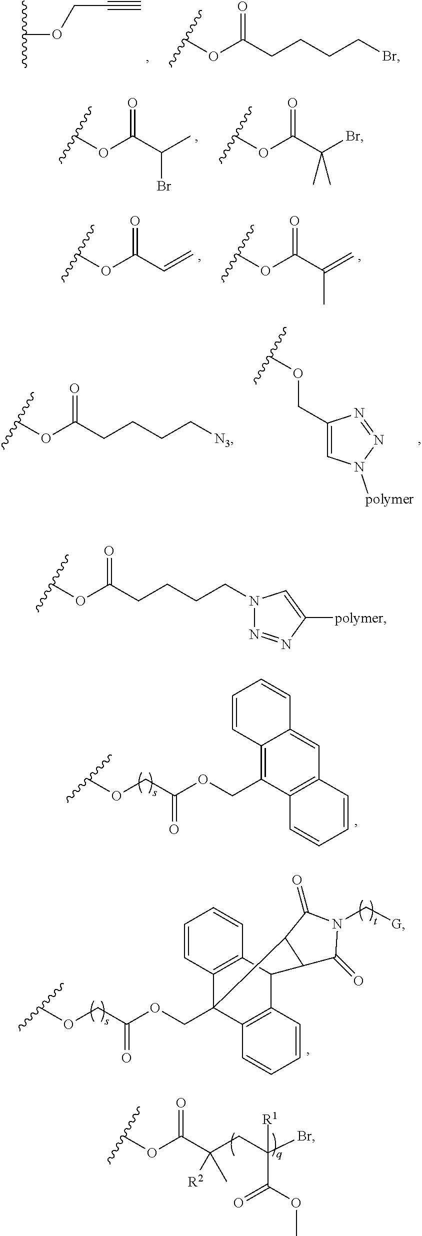

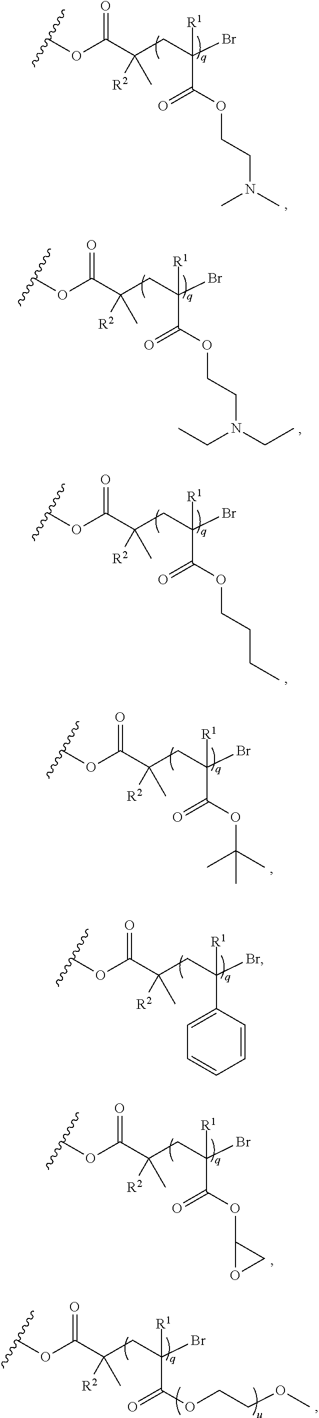

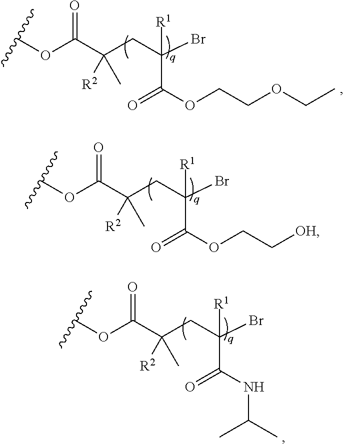

or a polymer;

### V is

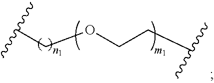

each D may be the same or different and is

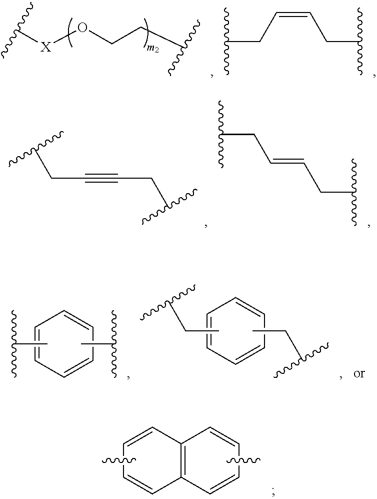

each n₁ may be the same or different and is an integer between 2 and 10;
each m₁ may be the same or different and is an integer between 0 and 20;
each X may be the same or different and is C₂-C₁₀ alkyl;
n₃ is an integer between 2 and 10;
R¹ is H or CH₃;
R² is H or CH₃;
R⁴ is aryl, alkyl, or a polymer;
R⁵ is aryl, alkyl, or a polymer;
R⁷ is H or halogen;
p is an integer between 3 and 200;
q is an integer between 1 and 100;
r is an integer between 0 and 100;
s is an integer between 1 and 10;
t is an integer between 1 and 10;
u is an integer between 1 and 100; and
G is a polymer, aryl, or alkyl.

In another aspect, the invention is directed to a class of compounds of formula (IV)

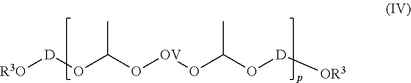

wherein,

### R³ is

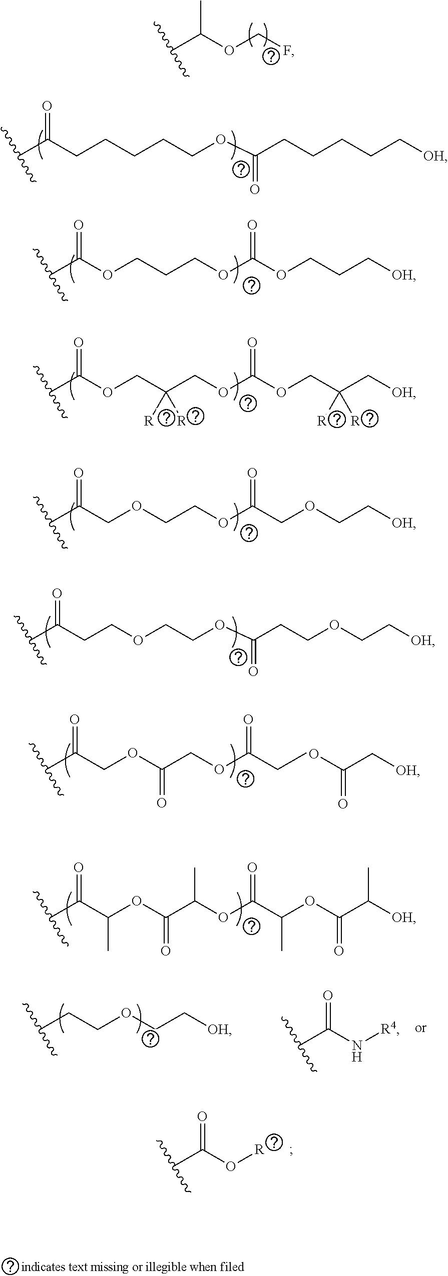

### F is

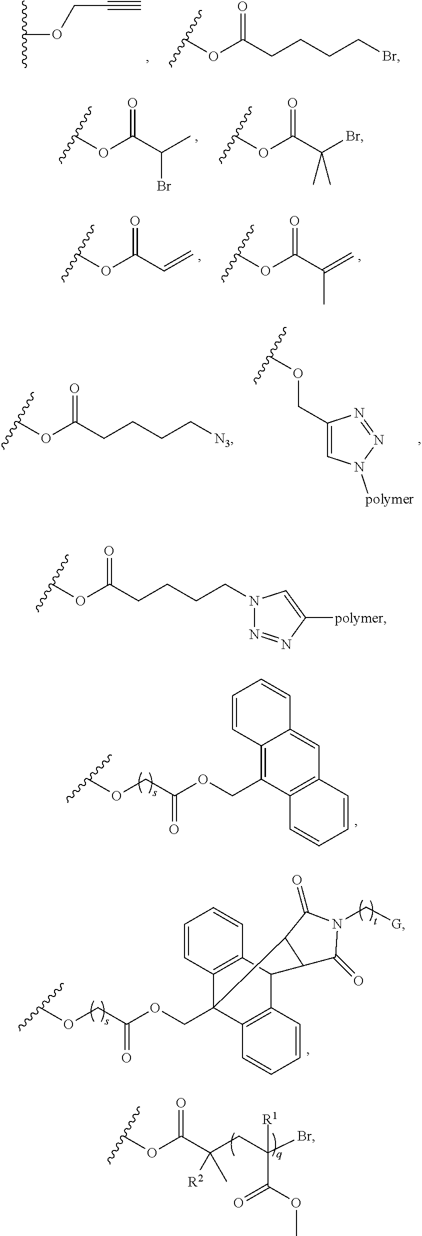

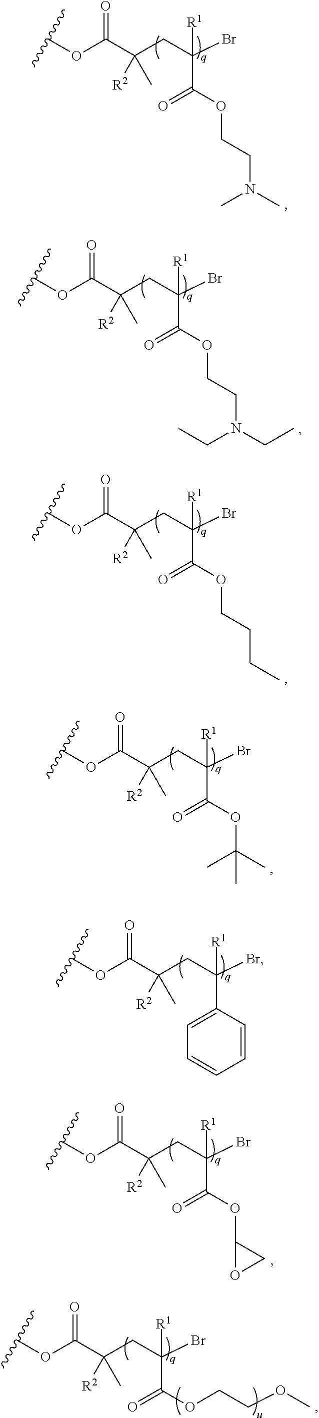

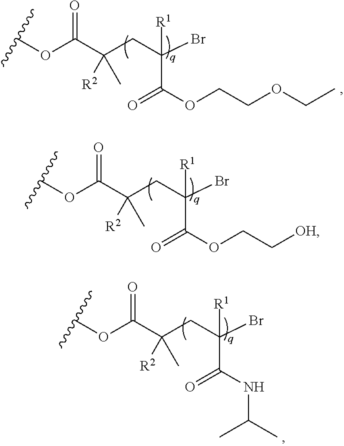

### V is

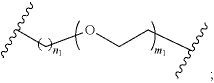

each D may be the same or different and is

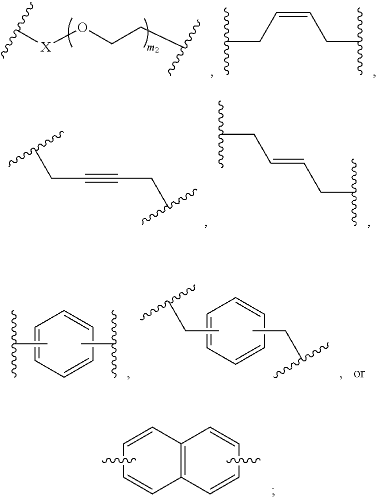

or a therapeutic agent core;
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
n₃ is an integer between 2 and 10;
R¹ is H or CH₃;
R² is H or CH₃;
R⁴ is aryl, alkyl, or a polymer;
R⁵ is aryl, alkyl, or a polymer;
R⁷ is H or halogen;
p is an integer between 3 and 200;
q is an integer between 1 and 100;
r is an integer between 0 and 100;
s is an integer between 1 and 10;
t is an integer between 1 and 10;
u is an integer between 1 and 100; and
G is a polymer, aryl, or alkyl.

In certain embodiments, a biodegradable gel comprising a compound of formula (IV) cross-linked with a linker, wherein the compound is cross-linked with a linker at a hydroxyl, alkyne or azide terminus.

In certain embodiments, a method of making a gel, comprising crosslinking a compound of formula (IV) with a trifunctional linker.

In certain embodiments, a micelle comprising a compound of formula (III):

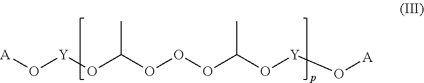

wherein

### A is

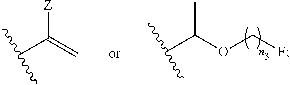

### F is

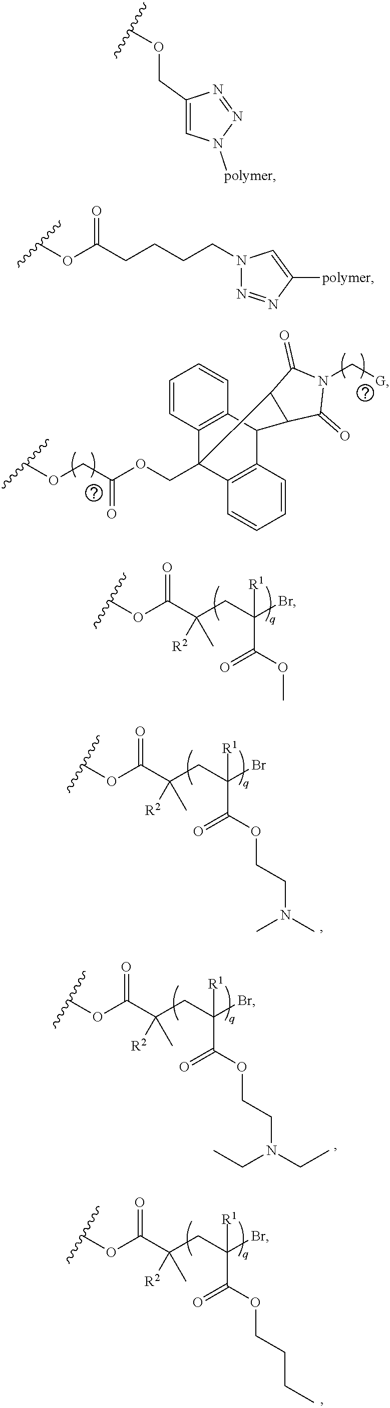

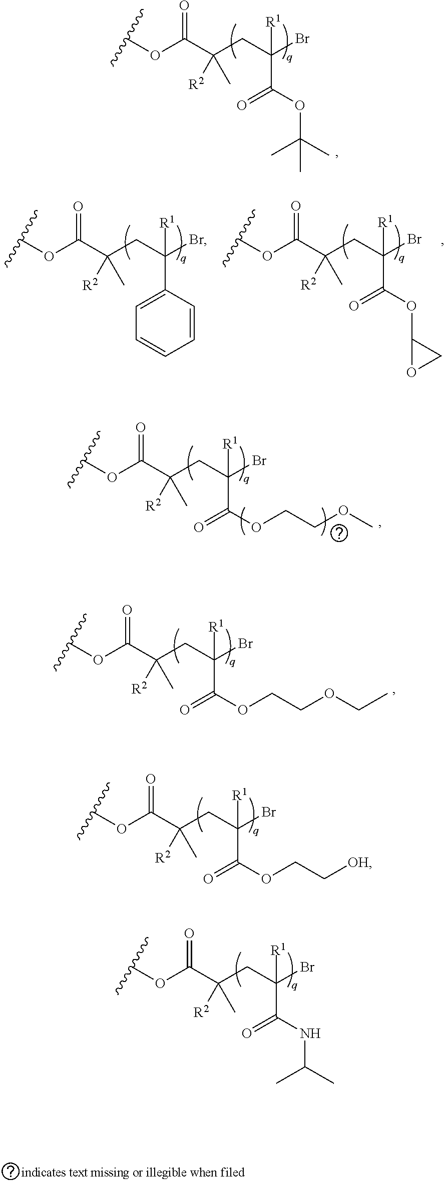

or a polymer;

### V is

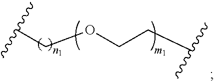

each d may be the same or different and is

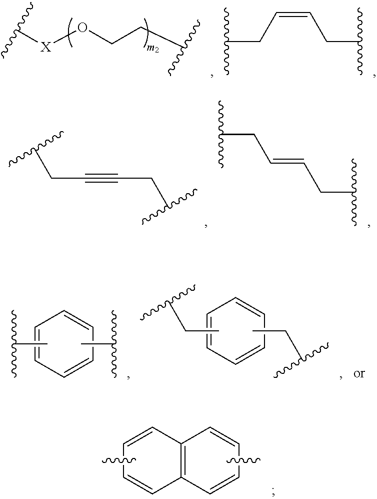

each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20; n₃ is an integer between 2 and 10;
G is a polymer, aryl, or alkyl;
Z is a polymer;
R¹ is H or CH₃; and
R² is H or CH₃;
p is an integer between 3 and 200;
q is an integer between 1 and 100;
s is an integer between 1 and 10;
t is an integer between 1 and 10;
u is an integer between 1 and 100;

a compound of formula (IV):

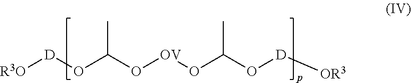

wherein

### R³ is

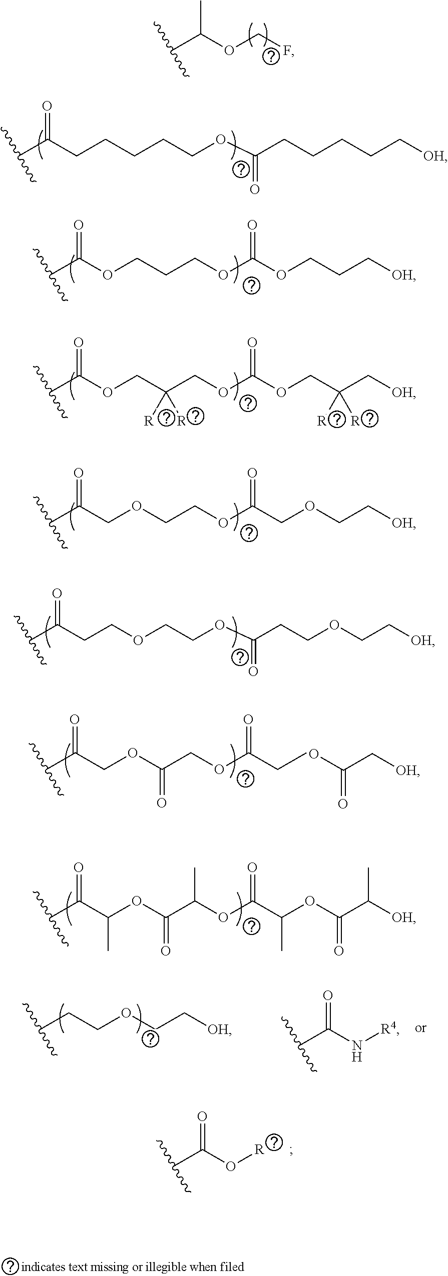

### F is

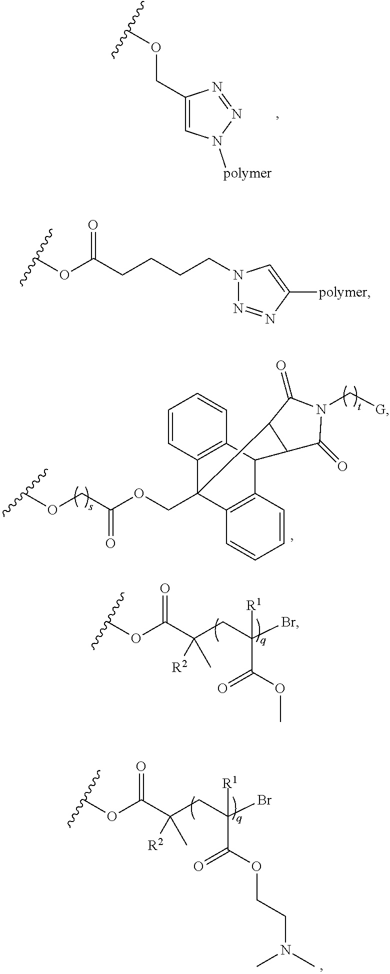

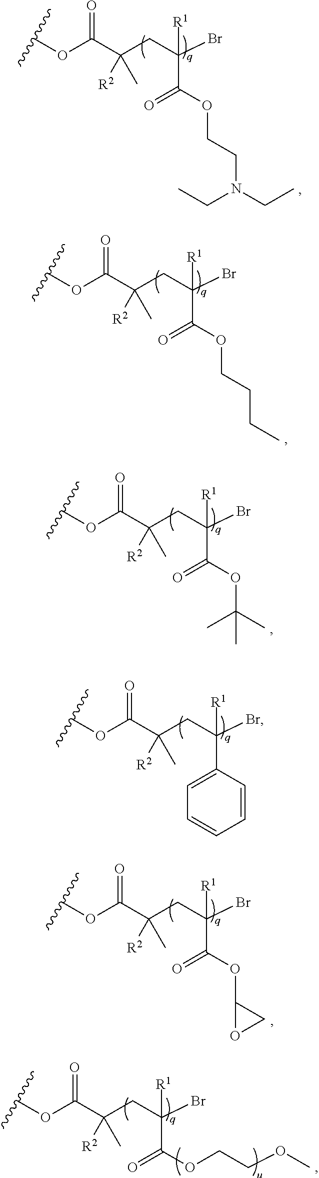

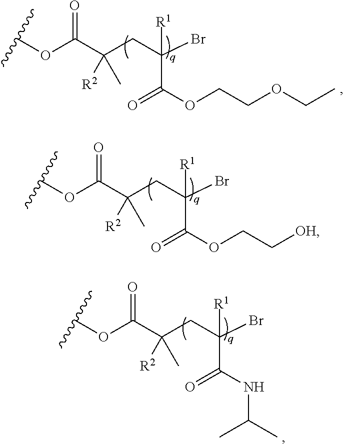

or a polymer;

### V is

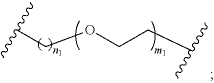

each D may be the same or different and is

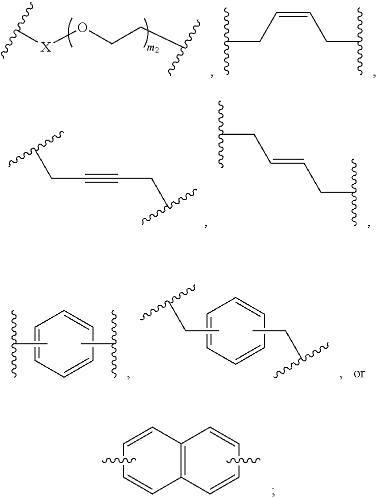

each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20; n₃ is an integer between 2 and 10;
G is a polymer;
R¹ is H or CH₃;
R² is H or CH₃;
R⁴ is a polymer;
R⁵ is a polymer;
R⁷ is H or halogen;
p is an integer between 3 and 200;
q is an integer between 1 and 100;
r is an integer between 0 and 100;
s is an integer between 1 and 10;
t is an integer between 1 and 10; and
u is an integer between 1 and 100.

In certain embodiments, a pharmaceutical composition comprising a micelle comprising a compound of formula (III) or (IV).

In certain embodiments, a class of compounds of formula (V):

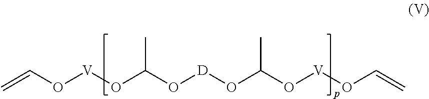

wherein,

### V is

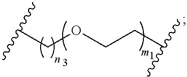

each D may be the same or different and is

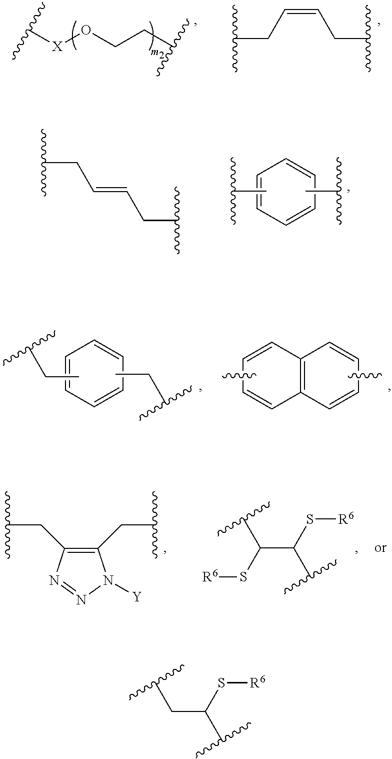

each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
p is an integer between 3 and 200;
Y is a polymer or therapeutic agent; and
R is alkyl, aryl, or a polymer.

In certain embodiments, a biodegradable gel comprising a compound of formula (V) cross-linked with a linker at a terminus of the compound, wherein the cross-linker is bonded to a plurality of compounds of formula (V).

In certain embodiments, a method of making a gel, comprising crosslinking a compound of formula (V) with a trifunctional linker.

In certain embodiments, a class of compounds of formula (VI):

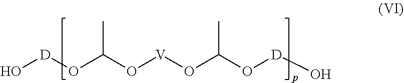

wherein,

### V is

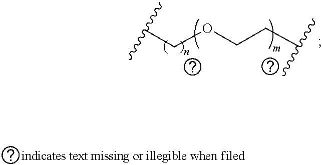

each D may be the same or different and is

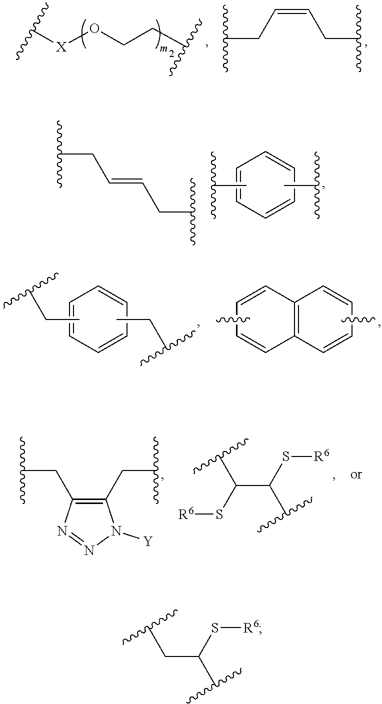

each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
p is an integer between 3 and 200;
Y is a polymer or therapeutic agent; and
R⁶ is alkyl, aryl, or a polymer.

In certain embodiments, a biodegradable gel comprising a compound of formula (VI), wherein the compound is cross-linked with a linker at a terminus of the compound; and wherein the cross-linker is bonded to a plurality of compounds of formula (VI).

In certain embodiments, a method of making a gel, comprising crosslinking a compound of formula (VI) with a trifunctional linker.

In certain embodiments, a method of delivering a therapeutic agent to a wound comprising, administering a biodegradable gel comprising a compound of formula (VI), wherein the compound is cross-linked with a linker at a terminus of the compound; and wherein the cross-linker is bonded to a plurality of compounds of formula (VI), and a therapeutic agent, wherein said gel degrades at pH from about 5 to about 6.5 to release said therapeutic agent.

In various embodiments of formulas (I) through (VI) described above, each n₁ may be the same or different and is an integer between 2 and 4; each m may be the same or different and is an integer between 0 and 2; each X may be the same or different and is C₂-C₅ alkyl or C₂-C₅ n-alkyl; each m₂ may be the same or different and is an integer between 0 and 3, or 2 or 3; and p is an integer between 3 and 100, between 3 and 200, between 10 and 200, between 10 and 100 or between 3 and 50.

In various embodiments of formulas (I) through (VI) above, q is an integer between 1 and 1000, between 1 and 500, between 1 and 100, between 100 and 1000, between 100 and 500, between 10 and 1000, between 10 and 500 or between 10 and 100.

In various embodiments of formulas (I) through (VI) above, r is an integer between 1 and 1000, between 1 and 500, between 1 and 100, between 100 and 1000, between 100 and 500, between 10 and 1000, between 10 and 500 or between 10 and 100.

In various embodiments of formulas (I) through (VI) above, s is an integer between 1 and 10, between 1 and 8, between 1 and 5, between 1 and 3, or 1 or 2.

In various embodiments of formulas (I) through (VI) above, t is an integer between 1 and 10, between 1 and 8, between 1 and 5, between 1 and 3, or 1 or 2.

In various embodiments of formulas (I) through (VI) above, u is an integer between 1 and 1000, between 1 and 500, between 1 and 100, between 100 and 1000, between 100 and 500, between 10 and 1000, between 10 and 500 or between 10 and 100.

In various embodiments, the value of [(m₁+m₂)/p] is a number between 0 and 8; or a number between 1 and 8; or a number between 0 and 6; or a number between 1 and 6; or a number between 0 and 4; or a number between 1 and 4.

In some embodiments, the trifunctional linker comprises one or more, or a plurality of any of the following: alkynes, alcohols, isocyanates or azides. In some embodiments, the trifunctional linker comprises three alkynes, three alcohols or three isocyanates. In some embodiments, the trifunctional linker comprises a triol. In some embodiments, the triol is glycerol or trimethylolpropane. In some embodiments, the trifunctional linker comprises three isocyanates.

In some embodiments, the cross-link comprises a urethane, triazole, or an ester.

In some embodiments, the cross-link comprises three urethane linkages, three ester linkages or three triazole linkages.

In some embodiments, the linker is linked to three compounds of any of formulas (I) through (VI) herein.

In some embodiments, the trifunctional linker comprises a tri-isocyanate. In some embodiments, the tri-isocyanate is triphenylmethane-4,4′,4″-triisocyanate, 1,3,5-cyclohexane triisocyanate, or 1,3,5-benzene triisocyanate.

In some embodiments, the cross-linker is linked to three polyacetals or polyketals.

In some embodiments, the cross-link comprises an acetal or a ketal. In some embodiments, the cross-link comprises three acetal linkages or three ketal linkages. In some embodiments, the cross-link forms acetal linkages or ketal linkages to a plurality of compounds of any of formulas (I) through (VI) herein.

In some embodiments, the linker forms acetal linkages or ketal linkages to one or more of compounds of any of formulas (I) through (VI) herein. In some embodiments, the linker comprises a triol or one or more triazoles, for example, three triazoles.

In some embodiments, the compounds exhibit a hydrodynamic radius of about 4.5 nm to about 75 nm, about 4.4 nm or about 75 nm.

In some embodiments, the compound has a lower critical solution temperature (LCST) from about 6° C. to about 80° C.; about 6° C. to about 70° C.; about 12° C. to about 70° C.; about 12° C. to about 38° C.; about 25° C. to about 50° C.; about 25° C. to about 45° C.; about 26° C. to about 43° C.; about 31° C. to about 43° C.; or about 37° C. to about 43° C.

In some embodiments, the m₍ₐᵥ₎ is from about 0.5 to about 2.5; or about 1.5 to about 2.5.

In some embodiments, the lower critical solution temperature transition occurs over a range of about 3-9° C.; over a range of about 3-5° C.; over a range of about 3-4° C.; over a range of about 3° C.; over a range of about 4° C.; or over a range of about 5° C.

In some embodiments, the transition temperature occurs over a range of about 3-9° C.; over a range of about 3-5° C.; over a range of about 3-4° C.; over a range of about 3° C.; over a range of about 4° C.; or over a range of about 5° C.

In some embodiments, the click functional macromonomers are poly-azide or poly-alkyne macromonomers. Poly-azide macromonomers can include any azide-terminated polymer. Exemplary poly-azide macromonomers include PEG-N3, PMMA-N3, NIPAM-N3, PDMAEDA-N3, PS-N3, PEO-N3, and PtBA-N₃. Other poly-azide macromonomers are disclosed, for example, in WO 10/053993, herein incorporated by reference in its entirety. Poly-alkyne macromonomers can include any alkyne-terminated polymer. Exemplary alkyne-terminated macromonomers include PEG-alkyne, PMMA-alkyne, NIPAM-alkyne, PDMAEDA-alkyne, PS-alkyne, PEO-alkyne, and PtBA-alkyne.

Triblock copolymers may include any ABA-type polymer wherein the B-block is a polyacetal or a polyketal. Exemplary triblock copolymers include PEG-polyacetal-PEG, PMMA-polyacetal-PMMA, PEO-polyacetal-PEO, NIPAM-polyacetal-NIPAM, PDMAEDA-polyacetal-PDMAEDA.

In some embodiments, the polymer is PEG, PMMA, PEO, NIPAM, PDMAEDA, PS, or PtBA.

In some embodiments, the therapeutic agent is a protein, peptide, drug, agricultural agent, small molecule therapeutic, antitumor agent or carbohydrate.

In some embodiments of any of formulas (I) through (VI), the therapeutic agent is a protein, peptide, drug, or carbohydrate.

In some embodiments, the polyacetal or polyketal compounds (PAs) herein show a number of advantageous and unique properties and behaviors that distinguish them from existing temperature responsive or pH-degradable polymers. For example, polyacetals are produced by reactions complete within about 2 hours. The polyacetal compounds are also the first water-soluble polymers that are intrinsically both pH-degradable and temperature responsive, with LCSTs bracketing body temperature. LCST transitions are sharp; copolymers need not be prepared to introduce degradation sites. PAs studied herein show no hysteresis in their LCST behavior. LCSTs do not depend strongly on either salt or polymer concentration. LCSTs can be controlled and predicted over essentially all practical temperatures for aqueous solutions (e.g., 6-80° C.), by using a mixture of two different diol monomers. PAs have a degradation mechanism that produces neutral products, whereas many polymers degrade to produce acidic products that can cause inflammation. In addition, aqueous PA solutions are biocompatible.

In some embodiments of any of formulas (I) through (VI) herein, the therapeutic agent core can be any of the following:

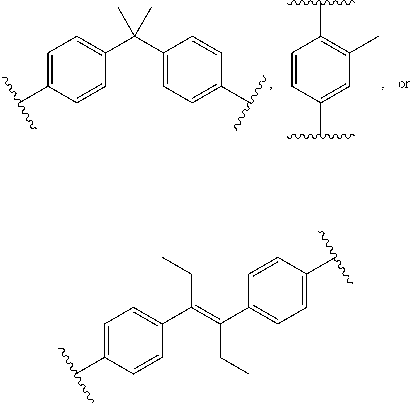

In various embodiments of formulas (I) through (VI) herein, the sum of (m₁+m₂) is greater than zero.

In some embodiments, the compound comprises a “drug core.”

In certain embodiments, the compositions herein include a polymer, and the polymer can be polystyrene, poly-t-butyl acrylate, polymethyl methacrylate or polyethylene glycol.

The above polymers are advantageous, in that they can permit release of active ingredients under specific conditions—for example, temperature range or pH range. Thus, a desired release rate can be achieved by customizing the compositions and relative amounts of the polymers. In certain embodiments, the technology herein provides a method for controlling the rate or amount of release of an antimicrobial agent, wound care agent, or any other therapeutic agent, onto a surface wound or surface of a medical device, by selecting one or more polymers known to have a certain characteristic that affects the rate of release of the agent—including, for example, a certain concentration or range of concentrations for which the polymer degradation profile matches the desired release profile or using a polymer with the desired release profile.

Antimicrobial Agent

In certain embodiments, the compositions herein include one or more antimicrobial agents—for example, impregnated into the polymers, or mixed with the polymers, or in one or more layers separate from the polymer. In certain embodiments, an antimicrobial agent can be applied first on the wound and then other components of the composition on top of the antimicrobial agent.

The antimicrobial agents can be any of those typically used either systemically or in wound care and treatment—including but not limited to: silver salts (e.g., silver sulfadiazine, silver nitrate, silver oxide, silver carbonate), chlorhexidine or its salts, benzalkonium chloride, povidone iodine, nitrofurazone, miconazole, bacitracin, neomycin, polymyxin, gentamicin, mupirocin, dicloxacillin, a cephalosporin (e.g., cephalexin, cefuroxime), clindamycin, erythromycin, bactroban, gentamicin or gentian violet; N-acetyl-L-cysteine or one or more alkanediols. The antimicrobial agents can also include, for example, fungicides (e.g., those used to treat toenail fungal infection, oral or vaginal fungal infection, or skin fungal infection); or agents used to treat acne (e.g., as a spot treatment to the skin). In certain embodiments, the films herein can incorporate one or more antifungal agents and can be applied either directly to a nail or incorporating it into nail polish or any other material then applied to the nail.

Other useful antimicrobial agents include any of the following botanical antimicrobial agents: essential oils and botanical extracts, e.g., orange oil, lemon oil, lemongrass oil, basil oil, rosemary oil, thymol, marjoram oil, fenugreek oil, tea tree oil, cranberry seed oil, menthol, camphor, cinnamon bark oil, amica flower oil, neem oil, tetrahydrocurcumin, lavender oil, lemon oil or extract, grapefruit seed extract, pomegranate oil or extract, aspenbark extract, wasabi extract, honeysuckle extract, sandalwood extract, black currant extract, benzoic acid, benzyl alcohol, berberine or phenylethanol.

Therefore, the compositions herein can contain both botanical and non-botanical antimicrobial agents, or one or the other. In various embodiments, the compositions herein contain one or more antimicrobial agents in amounts of 0.005 to 10%, 0.005 to 7.5%, 0.005 to 5%, 0.01 to 2%, 0.01 to 1% or 0.05 to 1%. In various embodiments, these percentages may describe either the total amount of antimicrobial agent, or the amount of antimicrobial agent separate from the further inclusion of a botanical antimicrobial agent in amounts of, e.g., 0.01 to 5%, 0.01 to 2%, 0.05 to 2%, 0.05 to 1%, 0.1 to 5% or 0.1 to 1%.

Wound Healing Agents

In certain embodiments, the compositions herein comprise a wound healing agent. These can include, e.g., aloe extract, aloe gel or powder, oat powder, oatmeal, oil, oat beta glucan, calendula oil, calendula extract, curcumin, Ginger extract, Rosemary oil or extract, Mango butter, Nutmeg butter, zinc salt, or witch hazel. In various embodiments, the compositions herein contain wound healing agent in an amount of 0.1 to 10%, 0.1 to 7.5%, 0.2 to 5%, 0.2 to 2.5%, 0.05 to 0.5, 0.01 to 0.3, 0.1 to 1% or 0.2 to 1% of the compositions.

Other Ingredients

In certain embodiments, the compositions further comprise an emulsifier. Exemplary emulsifiers include, e.g., oil-in-water emulsifiers and water-in-oil emulsifiers, liquid emulsifiers, solid emulsifiers, instant cold emulsifiers and emulsifiers for sprays (also known as solubilizers). In certain embodiments, useful emulsifiers include those known as polysorbates; including: polyoxyethylene sorbitan (20) monooleate (Polysorbate 80), Polyoxyethylene (20) sorbitan monolaurate (Polysorbate 20) polyoxyethylene (20) sorbitan monopalmitate (Polysorbate 40), Polyoxyethylene (20) sorbitan monostearate (Polysorbate 60), Polyoxyethylene (2) sorbitan tristearate (Polysorbate 65). Other suitable emulsifiers include, for example, glyceryl stearate and PEG 100 stearate (available commercially under the trade name Arlacel 165 from Croda (United Kingdom)); or sorbitan oleate (available commercially under the trade name Span-80 from Croda (United Kingdom)); or emulsifiers available under the trade name Polawax™ from Croda (United Kingdom). In various embodiments, the emulsifier is present in amounts of 0.1 to 10%, or 0.5 to 10%, or 1 to 5% of the compositions herein.

In certain embodiments, the compositions further contain any of the following: a solvent, an emollient, or a carrier. Exemplary solvents include, e.g., tetrahydrofuran (THF), ethanol, methanol or combinations thereof. In certain embodiments, inclusion of an emollient solvent can be advantageous. Emollient solvents include alkanediol (for example, methanol or ethanol), phenoxyethanol, benzyl alcohol, ethyl hexyl glycerin, propylene glycol, dipropylene glycol, glycerol, diglycerol. In various embodiments, any one or more solvents, emollients or carriers can be present in an amount of 0.1 to 10%, 0.2 to 5%, 0.2 to 2% or 0.3 to 2%, 1 to 5%, 5 to 20%, 5 to 30%, 20 to 80%, or 50 to 70%. In certain embodiments, a film forming composition herein comprises petrolatum, also known as petroleum jelly (for example, in amounts of 0.5 to 5% or 2 to 8%).

In certain embodiments, the compositions herein contain water; for example, in amounts of 1 to 90%, 5 to 80%, 10 to 70%, 15 to 60% or 20 to 40%. In certain embodiments, once the ingredients are included, water is added to the compositions herein to achieve 100% (that is, q.s. to 100%).

In other embodiments, they contain essentially no water (that is, less than 1%, or 0% water).

In certain embodiments, the compositions herein can contain any of the following:

A fruit acid (for example, mandelic acid), in amounts of 0.1 to 5%, 0.1 to 2%, 0.5 to 5% or 0.5 to 3% or 0.5 to 2%.

Lactic acid, in amounts of 0.01 to 1%, 0.01 to 2%, 0.05 to 2% or 0.5 to 2%.

A silicone adhesive (for example, that available under the trade name MD7-4502 from Dow Corning of Midland, Mich., USA); or that available under the trade name A-100 from Factor 2, Inc. of Lakeside, Ariz., USA) in amounts of 1 to 5% or 0.1 to 5% or 0.2 to 5% or 0.01 to 5%.

A urethane adhesive (for example, that available under the trade name Loctite M-06FL from R. S. Hughes in Sunnyvale, Calif., USA) in amounts of 0.1 to 15%, or 1 to 15%, or 1 to 10%.

Propanediol (for example, 1,3 propanediol available under the trade name Zemea from DuPont Tate & Lyle BioProducts of Loudon, Tenn., USA), in amounts of 0.1 to 10%, 0.5 to 10% or 1 to 10%.

Thus, in certain embodiments, the present technology is directed to a film forming triple polymer composition that can be prepared as follows:

(a) 5 to 30% pH-degradable polyacetal co-polymer or polyacetal-octanediol (or other) conjugate, or pH-degradable polyketal co-polymer or polyketal-octanediol (or other) conjugate;

(b) 0.5 to 5% chitosan-derived hydrophilic polymer;

(c) 0.2 to 5% hydroxypropylmethylcellulose stearoxy ether.

Coating Compositions

In certain embodiments, the compositions herein can be in the form of coating compositions for medical devices. For example, insertable medical devices such as catheters, stents, trocars or intravenous tubes can be coated with, or dipped into, the compositions herein. In certain embodiments, those described herein are referred to as triple polymer composition or complex (FTP) and can, in certain embodiments, contain a mixture of pH-degradable polyacetal co-polymer or polyketal copolymer, or polyacetal-octanediol conjugate or polyketal-octanediol conjugate (e.g., 1 to 20% w/w); and polyurethane polymer (e.g., 0.2 to 5% or 1 to 10%, or 2 to 10%, or 1 to 5% of either of a first polyurethane composition or a second polyurethane composition); and one or more antimicrobial agents discussed herein. In various embodiments, the compositions can also include one or more organic acids.

Catheter associated urinary tract infection (CAUTI) is the most common hospital associated infection (HAI) accounting for 42% of all HAIs and leading to about 80,000 deaths annually. Between 15 and 25% of patients hospitalized in the US now receive urinary catheter (UC) and almost 85% of intensive care patients require UCs. The annual cost of hospitalization from indwelling catheter related infection is estimated to be $1.3 billion in US and about $45 million in India. The incidence of CAUTI has quadrupled over the past decade and is expected to further increase with the projected rise in number of elderly patients requiring catheterization. It is well documented that most of the common uropathogens develop biofilm intraluminally or extraluminally in urinary catheters. A technological innovation that may prevent biofilm formation is a logical goal for reducing risk of CAUTI.

Commercially available antibacterial latex UCs coated with silver alloy hydrogel has debatable clinical value and cost effectiveness. In clinical studies, silver impregnated UC has shown only a minimal effect resulting in an increased cost of 80-130% over uncoated UCs. Despite this higher cost, about 40% of hospitals currently employ antimicrobial UCs. This indicates there is a clear need and demand for new, cost effective device coating technologies that are more effective in preventing CAUTI. Although silver is an effective antimicrobial agent, inability to release active amounts of silver from currently existing silver catheters has resulted in their lower efficacy in clinical settings.

Recent reports have shown that for some patients, allergic reaction to chlorhexidine has been noted. The FDA has suggested adding a warning in the label about possible chlorhexidine allergy. Therefore, a need exists for coating compositions that are free of chlorhexidine. In certain embodiments herein, the compositions are chlorhexidine-free (meaning that, in various embodiments, they contain zero chlorhexidine, or less than 1%, less than 0.1% or less than 0.01% chlorhexidine).

In various embodiments, the medical devices that can be coated herein include but are not limited to: catheters (e.g., a central venous catheter or peritoneal dialysis catheter) or endotracheal tubes or wound dressings or hernia patches comprising polyurethane, silicone, Dacron®, polytetrafluoroethylene (PTFE) or polyvinyl chloride (PVC) or any synthetic or natural polymer including cotton; or biomedical polymers comprising any of these.

Mechanism of FTP-M-AgSD Coated Medical Devices

In certain embodiments herein, both chlorhexidine-free and chlorhexidine containing antimicrobial coating compositions have been developed, as well as coating compositions containing chlorhexidine and other agents that reduce inflammatory reaction from coated medical devices. In certain embodiments, these compositions comprise 2 types of polyurethane—that is, a first polyurethane composition and a second polyurethane composition (e.g., Tecoflex® 93A and Tecoflex® 60D); a degradable polyacetal polymer, decanediol (for example, 0.5 to 20% or 1 to 3%) and silver salts (FTP-M-AgSD) or chlorhexidine (FTP-M-CHX) for coating medical devices including urinary catheters, central venous catheters, endotracheal tubes and the like.

The agents that reduce the inflammatory response used herein include zinc salts such as zinc gluconate, zinc lactate, zinc salicylate, zinc acetate, zinc citrate (for example, 0.1 to 2%, 0.1 to 1% or 0.01 to 2%), and any combination thereof. Zinc salts have been shown to reduce latex related allergy. In certain embodiments, witch hazel, panthenol, calendula oil, aloe gel, rosemary oil (for example, 0.1 to 1%) or combinations thereof can be further included to reduce the inflammatory response. These agents can also be used in the presence of silver sulfadiazine.

In certain embodiments, the technology related to the coatings herein is tailored to have a unique antimicrobial surface ideal for biofilm resistant antimicrobial medical device—that is, the device prevents adherence of bacteria and biofilm formation on its surface, but does not release therapeutic amount of antimicrobials to prevent systemic infection. In such embodiments, the daily release is marginal but sufficient to inactivate pathogens introduced initially from the insertion site around the catheter. Most of the antimicrobials remain on the lubricious FTP-M-AgSD surface for a prolonged period of time, helping to prevent subsequent microbial adherence and biofilm formation. The active ingredients can then be eluted in a prolonged time so as to achieve a longer duration of antimicrobial efficacy. Furthermore, this lubricious polymeric surface especially on the urinary catheter meet the need for an ideal catheter which makes insertion easier and thus providing more comfort to the patient avoiding the need for an overcoat with hydrogels. Current practice to improve lubricity of the catheter surface is using a hydrogel overcoat. This extra step is time consuming and costly. However, using the technology herein is advantageous in permitting the elimination of the need for a hydrogel overcoat.

PA-OCT Composition for Medical Devices and Wound Care

The PA-OCT composition (also referred to as the “PA-OCT polymer” that is referred to herein comprises a polyacetal conjugate that is part tetraethylene glycol and part 1,8-octanediol (a biodegradable or thermoresponsive polymer such as those disclosed in International Patent Application No. PCT/US2015/063669, published as WO/2016/090103 on Jun. 9, 2016). “PA-OCT-80” refers to the ratio of tetraethylene glycol:1,8-octanediol, which in this case is 20:80. Similarly, “PA-OCT-75,” “PA-OCT-50” and so forth can be prepared by adjusting the ratio of tetraethylene glycol:1,8-octanediol accordingly.

In certain embodiments, the compositions herein are maintained at a pH that is fairly close to neutral—that is, 5 to 9, 5.5 to 8.5, 6 to 8, 6.5 to 7.5, 6.8 to 7.20 or 7.

In certain exemplary embodiments, the present technology is directed to a hydrophilic film forming wound healing topical cream or gel containing one or more wound healing agents, where the one or more wound healing agents are any of the following: (a) flaxseed oil and its derivatives (for example, 0.1 to 5% or 1 to 5%); (b) calendula oil or extract (for example, 0.3 to 1%); (c) aloe vera gel (for example, 0.1 to 0.5% or 0.1 to 5%) or combinations thereof. In certain embodiments, a composition can further contain any of the following: antimicrobial agents such as silver sulfadiazine (for example, 1%) or silver oxide (for example, 0.02 to 0.3%); Neosporin® antimicrobials (Neomycin, Polymyxin B, Bacitracin combinations). In certain embodiments, povidone iodine or the like can also be included to control infection as well as enhance wound healing properties.

Preliminary results in rat model showed 30 to 50% enhanced wound healing with the topical application of wound healing cream alone as compared to commercial creams on wound created on dorsum of rats. Incorporation of antimicrobial agents in this wound healing cream can offer an advantage to wound healing over using an antimicrobial agent alone.

In certain embodiments, a film forming triple gel composition (FTP) was also prepared, comprising poyacetal-octanediol (PA-OCT) conjugate, a hydrophilic film forming polymer (e.g., chitosan), and a hydrophobically modified hydroxypropyl methylcellulose modified with an ethoxy group (e.g., Sangelose®). The FTP composition contained emlientsolvent s decanediol, and wound healing agents which were one or more of the following: (a) flaxseed oil and its derivatives (0.5 to 5% or 1 to 5%); (b) calendula oil or extract (0.3 to 1%); (c) aloe vera gel (0.1 to 5%) or combinations thereof. Antimicrobial agents such as silver sulfadiazine (1%), silver oxide (0.02 to 0.3%) and Neosporin® actives (Neomycin, Polymyxin B, Bacitracin combinations), povidone iodine and the like were also incorporated into FTP.

In various embodiments discussed herein, the film forming compositions discussed herein comprise the FTP in amounts of, for example, 0.1 to 2%, 0.1 to 5%, 0.2 to 5%, 0.5 to 5%, 1 to 5% or 1 to 10% of the overall film forming composition.

### Example 1

### Film Forming Triple Polymer Composition for Wound Care (FTP-W)

A film forming triple polymer (FTP) gel composition was prepared as follows:

(a) 5 to 30% pH degradable polyacetal polymer or polyketal polymer (PA) or any polyacetal-active or polyketal-active conjugate including polyacetal-octanediol conjugate or polyketal-octanediol conjugate (PA-OCT);

(b) 0.5 to 5% hydrophilic polymer (e.g., chitosan and derivative)

(c) 0.1 to 5% amphiphilic polymer including hydroxypropyl methylcellulose stearoxy ether (e.g., available under the trade name Sangelose®)

The PA and PA-OCT in this composition formed a film and permitted the controlled release of antimicrobials. Chitosan and Sangelose® gels were prepared in water to form hydrogels and used in this composition. These gels rendered the composition, less rigid, smooth and easily spreadable while allowing higher initial release of antimicrobials initially.

The FTP gel combination was mixed with 2 to 8% petrolatum (petroleum jelly); the petrolatum was used in this composition as an emulsifier. Other emulsifiers such as any of the polysorbates can be added in addition to (or instead of) petrolatum.

The final general combination of all parts (including antimicrobials mentioned below) is abbreviated FTP-W.

Table 1A shows some exemplary formulations tested. Numbers are expressed as percentage (w/w).

Table 1A General formula of FTP-W

TABLE 1A

| Ingredient | % Range |
|---|---|
| Petrolatum | 2 to 10 |
| Chitosan (3%) | 20 to 60 |
| Sangelose ®-90L (1%) | 15 to 40 |
| Tween 80 | 1 to 5 |
| PA4020 or PA-OCT | 2 to 20 |

### Example 2

### FTP-W Composition Containing Antimicrobials

To make up the complete formulation, 60 to 65% of the FTP composition was blended with antimicrobials and made to 100% with water or alcohol. The resulting polymer-antimicrobial blend was a viscous gel that could be spread as a film on skin.

The following FTP compositions containing wound healing agents and antimicrobials were prepared and evaluated:

(1) Silver sulfadiazine (FTP-AgSD)

(2) Neosporin® (FTP-NP)

(3) Combination of silver salts and botanical actives (FTP-SB)

The efficacy of the above groups were compared to commercially available standard of care products in both the prescription and over-the-counter (OTC) spaces.

Commercial products were:
Band-Aid® Plus Antibiotic Adhesive Bandages (Polymexin B Sulfate 10,000 units, Bacitracin Zinc 500 units, Petrolatum base).
Rite Aid® First Aid Advanced Antibacterial Adhesive Bandages (Benzalkonium chloride 0.1%).
Neosporin® (Polymexin B Sulfate 10,000 units, Bacitracin Zinc 500 units, Neomycin base 3.5 mg equivalent—Cream-NP).
Silvadene® Cream (1% Silver sulfadiazine—Cream-AgSD).
Silver ion+Botanicals incorporated in an inactive Silvadene base (Cream-SB).

Results are shown below in Tables 1B through 1D:

FTP-W 1

TABLE 1B
FTP-W-SZB5-1 (FTP-AgSD)

| Ingredient | % w/w |
|---|---|
| Petrolatum | 4 |
| Chitosan (3%) | 32 |
| Sangelose ®-90L (1%) | 20 |
| Tween 80 | 1 |
| PA4020 or PA-OCT | 10 |
| Water | 23 |
| SZB5 | 10 |
| Total | 100 |

TABLE 1C
FTP-W-SB6-1 (FTP-SB)

| Ingredient | % w/w |
|---|---|
| Petrolatum (80%) | 4 |
| Chitosan (3%) - (80%) | 32 |
| Sangelose ®-90L (1%) - (80%) | 16 |
| PA4020 or PA-OCT | 10 |
| Water | 27 |
| AgNO3 (0.5%) | 2 |
| SB6 | 9 |
| Total | 100 |

TABLE 1D
FTP-W-NP-1 (FTP-NP)

| Ingredient | % w/w |
|---|---|
| Petrolatum | 3.2 |
| Chitosan (5%) | 15.99 |
| Sangelose ®-90L (1%) | 12.79 |
| Tween 80 | 0.8 |
| PA4020 or PA-OCT | 8 |
| Decanediol | 0.8 |
| Water | 30.38 |
| NP Blend | 28.04 |
| Total | 100 |

The results for studies conducted are shown below.

Results 1: Comparison of Zone of inhibition of FTP-W antimicrobial with antimicrobial cream

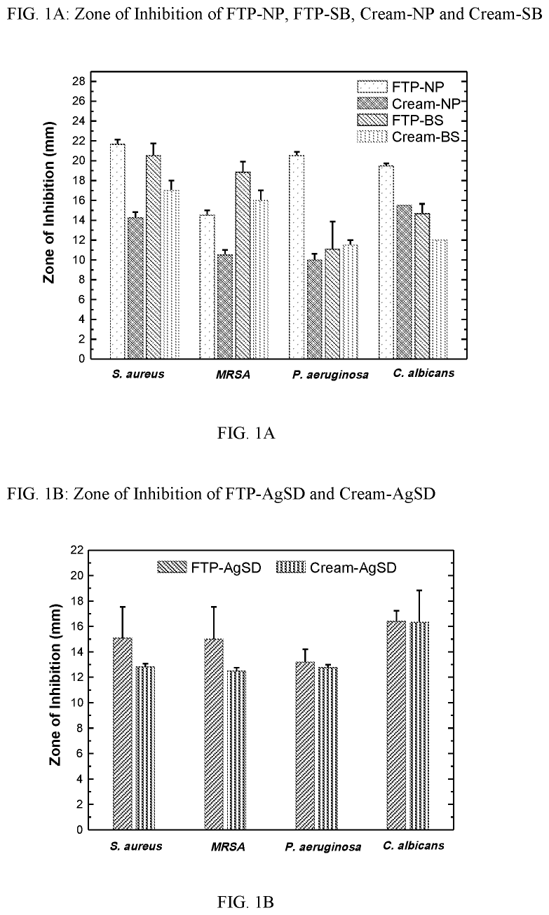

Method: Topical formulations of FTP-AgSD and FTP-SB and FTP-NP were evaluated for their initial antimicrobial efficacy against S. aureus, MRSA. P. aeruginosa and C. albicans using a diffusion well-plate method. Freshly prepared agar plate surfaces were inoculated by each of the above bacteria by spreading 0.3 ml of 10 CFUmL⁻¹ inoculum. A circular cavity of 6 mm was then cut out aseptically with a cork borer. For each well, 2 mL of FTP-W formulation was applied and placed in the incubator at 37° C. for 24 hours. For comparison, commercially available Cream-AgSD and Cream-NP were used as received from the pharmacy. The antimicrobial agent diffused into the agar medium and inhibited the growth of the microbial strain tested. This zone of inhibition was measured (subtracting the 6 mm diameter of the well), and results are shown in FIGS. 1A and 1B.

Conclusion: Incorporation of active ingredient into the FTP-W base was shown to improve the initial release of antimicrobials in almost all cases tested. The only exceptions were in FTP-BS on P. aeruginosa culture where Cream-BS showed marginally higher average efficacy than the FTP form. FTP-AgSD performed significantly better in S. aureus and MRSA but showed equal efficacy in P. aeruginosa and C. albicans.

Results 2: Comparison of zone of inhibition of FTP Band-Aid® compositions with commercial antibacterial Band-Aid® bandage

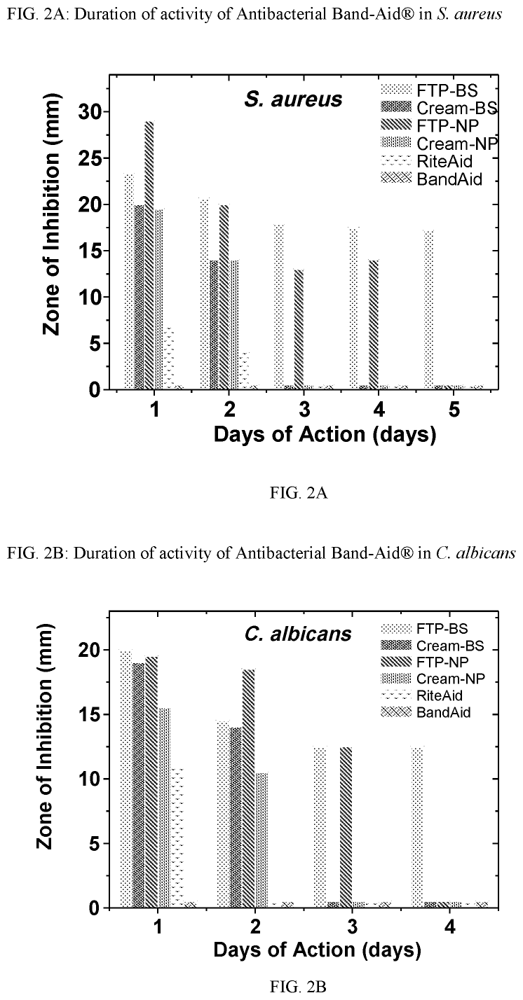

Method: FTP-W formulations and their respective cream counterparts were applied to commercially available uncoated Band-Aid® strips (1 cm×1 cm) and allowed to dry. Their antimicrobial efficacy was evaluated by placing these FTP-W and Cream coated Band-Aid® into bacteria inoculated agar plates and incubated at 37° C. for 24 hours. The zone of inhibition in each group was measured (subtracting the 8 mm length of the Band-Aid®). The duration of activity was determined by transferring the coated Band-Aid® daily to freshly seeded agar plates and measuring the zone of inhibition until there was no visible zone. FIG. 2A shows the duration of activity of the antibacterial Band-Aid® in S. aureus. FIG. 2B shows the duration of activity of the antibacterial Band-Aid® in C. albicans. FIG. 2C shows the duration of activity of the antibacterial Band-Aid® in P. aeruginosa.

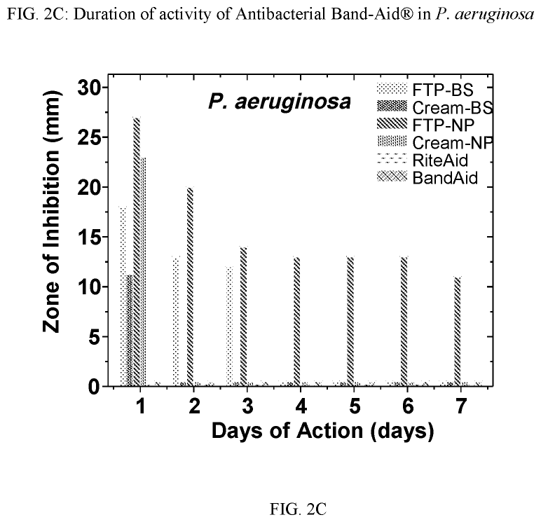

FIGS. 3A, 3B, 3C and 3D show the duration of activity of FTP-AgSD and Cream-AgSD applied on a Band-Aid® for S. aureus, P. aeruginosa, MRSA and C. albicans, respectively.

Conclusion: Delivery of commercial active ingredient through the FTP-W base improves the prolonged release of antimicrobials significantly in all cases tested.

Results 3: Retention of antibacterial agents in FTP-W coated and cream coated surface after several cycles of water rinse.

Method: The study was conducted by rinsing FTP-AgSD and Cream-AgSD with deionized water at 10 mL/second for temperatures above and below the LCST (10° C.). The off-white residues seen were AgSD (confirmed by zone of inhibition).

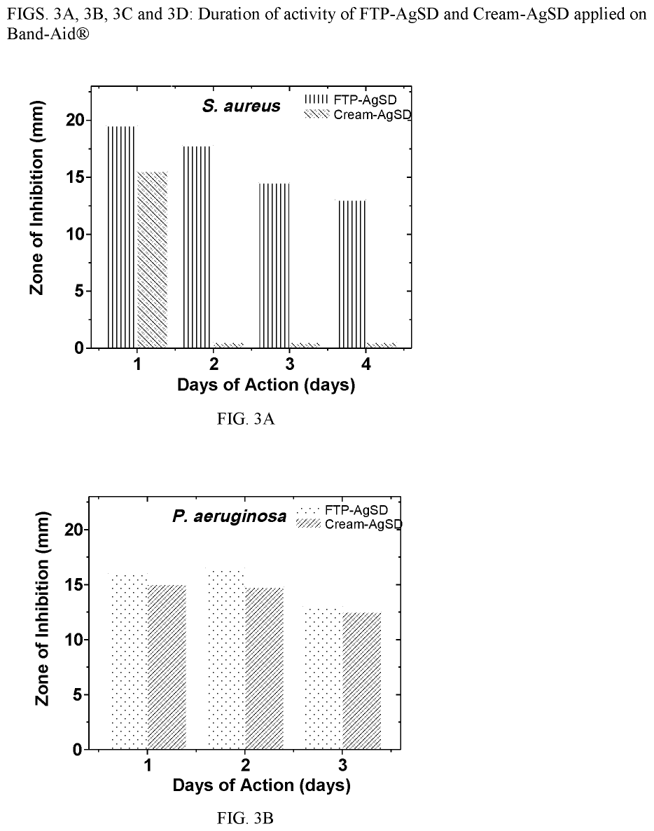

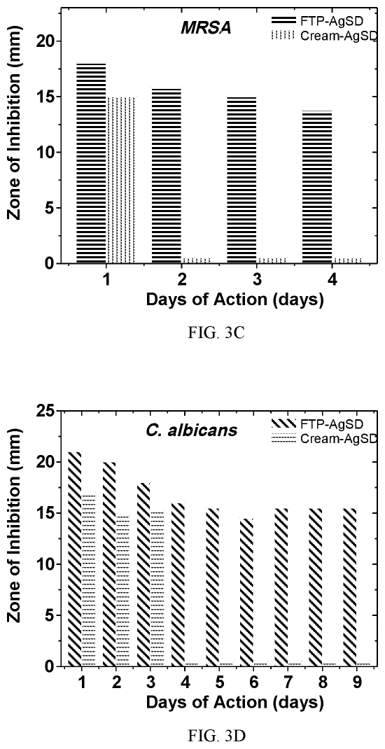

FIG. 4A shows the film retention time study of a) FTP-AgSD and b) Cream-AgSD at rinsing temperatures of 25° C. and 5° C. Retention of AgSD after rinsing was imaged and measured.

Conclusion: The FTP wound care system does not wash off at ambient conditions. However, at lower temperatures the film is removed easily. In contrast, Cream-A rinses off within 30 seconds at ambient conditions.

FIG. 4B shows Retention of antimicrobial efficacy after rinsing at 25° C. for a) 10% FTP-A, b) 15% FTP-A and c) Cream-A. The efficacy was measured using an ex-vivo pig-skin model. Test organism was S. aureus. The antimicrobial efficacy of FTP-A remains unchanged after 120 ml of rinsing. In contrast, Cream-A shows a steep decrease in efficacy at the same time.

Conclusion: The antimicrobial efficacy of FTP-A remains unchanged after 120 ml of rinsing. In contrast, Cream-A shows a steep decrease in efficacy at the same time.

Results 4: Optimization of PA/PA-OCT concentration in FTP

Method: Varied polyacetal concentrations (ranging from 0% to 5%, 10% and up to 20%) were tested against C. albicans. Commercially available inactive Band-Aid® bandages were treated with FTP-AgSD of varied polyacetal compositions and allowed to dry. C. albicans inoculated agar plates were used as substrate. Pieces of bandage were placed on the inoculated agar surfaces and incubated at 37° C. for 24 hours. The zone of inhibition (measured diagonally) was reported for several days. Each day, the bandage pieces were transferred to a freshly inoculated agar plate incubated as before.

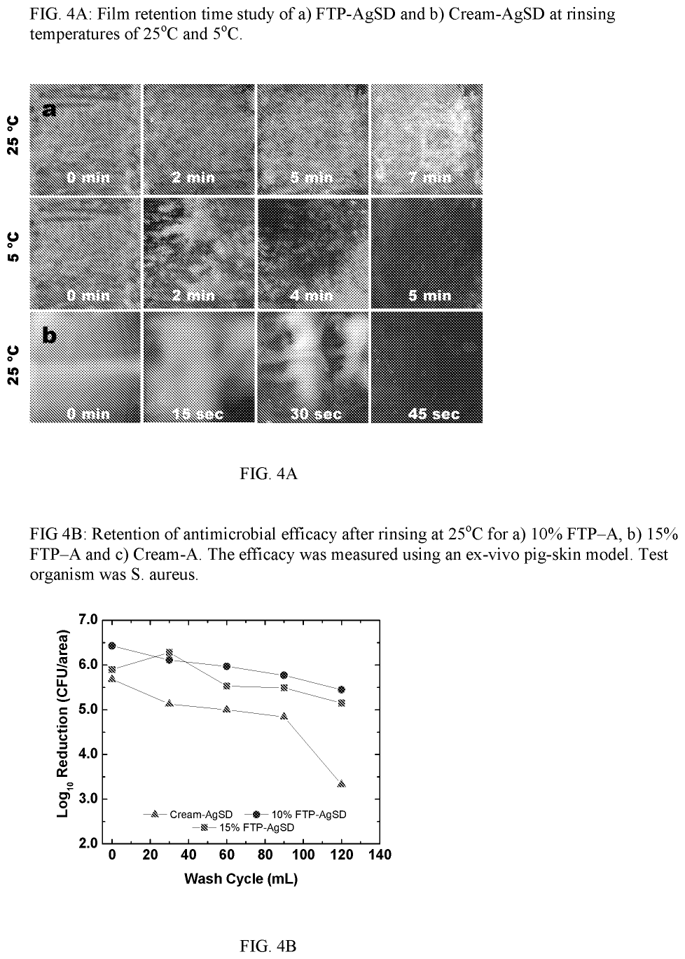

FIG. 5 shows the effect of polyacetal polymer concentration (in FTP) on the release of antimicrobial agent (AgSD) characterized by zone of inhibition: for optimization of polymer concentration. Groups tested were 5% PA₄₀₂₀V, 10% PA₄₀₂₀V, 10% PA-OCT, 20% PA-OCT and 0% PA.

Results 5: Evaluation of antimicrobial efficacy of antimicrobial FTP and antimicrobial cream using ex-vivo rapid kill test using porcine skin

Method: The ex-vivo porcine skin method was carried out according to ASTM E2897-12. Pre-prepared circular porcine skin (4.1 cm diameter) was adhered to a base and sterilized using 70% ethyl alcohol. For inoculation, 50 μL each of 107 CFU/mL bacteria was applied on a pair of pigskin followed by immediate rubbing of the two skins together for 15 seconds. The pigskin was then incubated in a humid environment (PBS) for 20 minutes for bacterial absorption. For test sample, 0.2 mL of FTP formulations were spread evenly with glass spreader until uniform layer was obtained. PBS was used as the control and was applied on the porcine skin in the same manner. The samples were allowed to incubate for 2 hours and 4 hours.

A circular cylinder (1 inch diameter) was placed over pigskin, and 1 mL of Drug neutralizing fluid (DE) added into the cylinder. The skin sample was scrubbed with scraper for 15 seconds and 9 mL of DE was added into the cylinder. The DE was mixed inside the cylinder and transferred to sampling tube.

The samples were serially diluted as required, spread over agar plates with a spreader and incubated at 37° C. for 24 hours. The colonies were counted and the log₁₀ reduction values were determined with respect to the control growth (PBS). The samples were tested in triplicate for each experiment and all experiments were performed three times.

Table 2 shows bacterial counts on pigskin treated with FTP-AgSD and Cream-AgSD (Test time: 2 hours and 4 hours).

TABLE 2

| Contact | | Log₁₀ Reduction (CFU/ml) | | |
|---|---|---|---|---|
| Time | Organism | S. aureus | P. aeruginosa | C. albicans |
| 2 hours | FTP-AgSD | 3.34 ± 0.70 | 2.25 ± 0.69 | 2.01 ± 0.33 |
| | Cream-AgSD | 2.56 ± 0.53 | 1.58 ± 0.67 | 1.43 ± 0.25 |
| 4 hours | FTP-AgSD | 2.83 ± 0.50 | 3.72 ± 0.13 | 2.39 ± 0.19 |
| | Cream-AgSD | 2.16 ± 1.03 | 2.86 ± 0.09 | 1.36 ± 0.01 |

Table 3 shows bacterial counts on pigskin treated with FTP-NP and Cream-NP (Test time: 2 hours).

TABLE 3

| Contact | | Log₁₀ Reduction (CFU/ml) | | |
|---|---|---|---|---|
| Time | Organism | S. aureus | P. aeruginosa | C. albicans |
| 2 hours | FTP-NP | 5.14 ± 1.38 | 3.00 ± 0.05 | 1.64 ± 0.20 |
| | Cream-NP | 3.15 ± 1.07 | 1.43 ± 0.06 | 0.42 ± 0.52 |

Table 4 shows bacterial counts on pigskin treated with FTP-SB and Cream-SB (Test time: 2 hours and 4 hours).

TABLE 4

| Contact | | Log₁₀ Reduction (CFU/ml) | | |
|---|---|---|---|---|
| Time | Organism | S. aureus | P. aeruginosa | C. albicans |
| 2 hours | FTP-SB | 2.33 ± 0.28 | 3.94 ± 0.54 | 3.26 ± 1.38 |
| | Cream-SB | 1.81 ± 0.10 | 1.45 ± 0.86 | 0.76 ± 0.23 |
| 4 hours | FTP-SB | 2.83 ± 0.50 | 3.72 ± 0.13 | Not Done |
| | Cream-SB | 2.16 ± 1.03 | 2.86 ± 0.09 | Not Done |

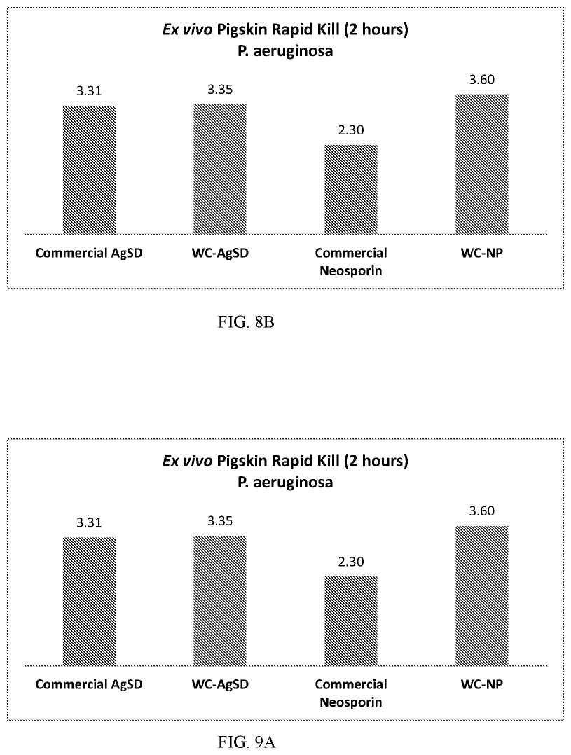

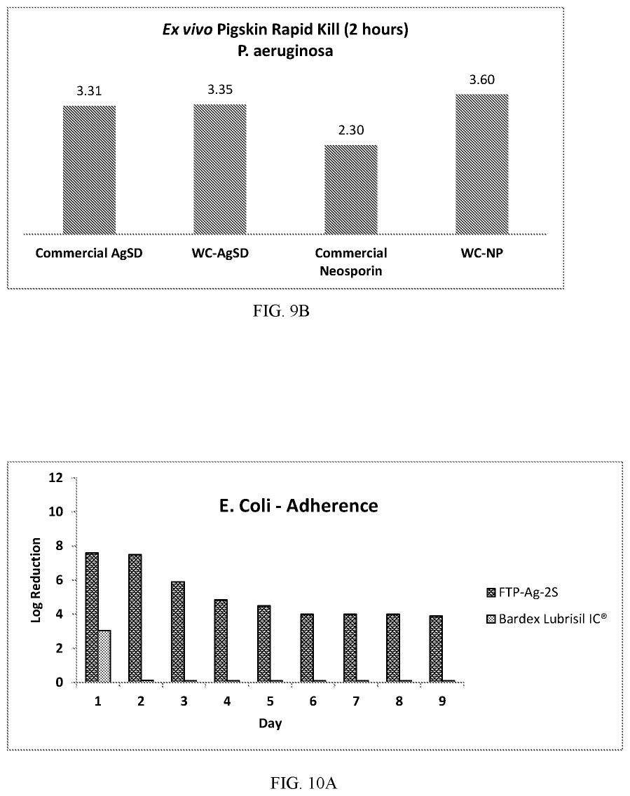

Results are also shown in FIGS. 8A-8B and 9A-9B. The following were observed:

1. WC-AgSD wound healing formulations show higher efficacy in ex vivo pig skin study when compared to commercially available AgSD cream for all pathogens tested.

2. WC-NP wound healing cream shows higher efficacy in ex vivo pig skin study when compared to commercially available Neosporin® wound healing cream.

Results also showed that the FTP-AgSD, FTP-NP and FTP-SB compositions, when applied directly on agar plates seeded with microorganisms, exhibited larger zones of inhibition against all organisms tested (S. aureus. P. aeruginosa and C. albicans) than cream containing 1% silver sulfadiazine or Neosporin® (triple antibiotic) or silver-botanicals.

When these compositions were applied on a Band-Aid® and tested for zone of inhibition in comparison with a commercial antibacterial Band-Aid®, the zones of inhibition were much larger against all the organisms, and the activity lasted for more than 4 days.

Silver sulfadiazine, Neosporin® and Silver botanical were incorporated in cream and FTP-W. They were evaluated for their efficacy on infected porcine skin, which was used as surrogate for human skin. The FTP-antimicrobial groups showed better efficacy than cream—antimicrobials silver sulfadiazine cream, in both 2 and 4 hours contact time studies.

The enhanced efficacy of FTP-W groups can be attributed to the following properties:
1: Chitosan and Sangelose® gels were shown to help release higher concentration of antimicrobials initially than that from the cream;
2: All of the experiments involving the FTP wound care compositions and the medical device coating compositions were conducted at a pH of 6.5 to 7. At pH values in this range, PA and its conjugates appear to degrade into its constituent monomers, allowing for antimicrobials to be released proportionally for a prolonged period. PA-OCT also greatly contributes to the film forming capability of the FTP-W.

The FTP-W compositions exhibited sustained broad spectrum antimicrobial efficacy for more than 4 days when tested by incorporation into a Band-Aid® bandage. In contrast, commercial antibacterial Band-Aid® bandages did not show broad spectrum activity or sustained activity. FTP-W compositions were also incorporated in a hydrophilic cream base and compared with commercial antibacterial topical creams. FTP-W topical creams showed higher broad spectrum activity.

FTP-AgSD can be used to treat burn wounds, diabetic ulcers, pressure wounds, as well as to prevent surgical site infection. This could be a better alternative to Silvadene® which is currently widely used in the treatment of burn wounds. Unlike Silvadene®, it is easy to apply and there is no need change the dressing and reapply a cream daily. Similarly, FTP-SB and FTP-NP could be over the counter products for treating minor skin infections. FTP-SB can also be used to coat food contact surfaces.

Some General Observations

The preliminary results show that FTP-NP and FTP-BS compositions when applied directly on agar plates seeded with microorganisms exhibit larger zones of inhibition against all the organisms tested (S.aureus, P.aeruginosa and C.albicans) than cream containing the same proportions of Neosporin or silver-botanicals.

When these compositions were applied on a Band-Aid® and tested for zone of inhibition in comparison with a commercial antibacterial Band-Aid®, the zones of inhibition were much larger against all the organisms and the activity lasted for more than 4 days.

Neosporin® and Silver botanicals were incorporated in cream and FTP-W. They were evaluated for their efficacy on infected porcine skin, which was used as surrogate for human skin. FTP-antimicrobial groups showed better efficacy than cream—antimicrobials silver sulfadiazine cream, in both 2 and 4 hours contact time studies.

We attribute the enhanced efficacy of FTP-W groups to the following properties:
1: Hydrogels help release higher concentration of antimicrobials initially than that of the cream.
2: All experiments were conducted at pH values of about 6.5 to about 7. At these pH values, PA and its conjugates degrade into its constituent monomers, allowing for antimicrobials to be released proportionally for a prolonged period. PA-OCT also greatly contributes to the film forming capability of the FTP-W.

Various FTP-W2 compositions described The FTP-W compositions exhibit sustained broad spectrum antimicrobial efficacy for more than 4 days when tested by incorporation into a Band-Aid. Commercial antibacterial Band-Aid do not show broad spectrum activity or sustained activity. FTP-W compositions were also incorporated in a hydrophilic cream base and compared with commercial antibacterial topical creams. FTP-SB and FTP-NP will be over the counter products for treating minor skin infections. FTP-SB can also be used to coat food contact surfaces.

### Example 3

Various FTP-W2 compositions described below were prepared as discussed below.

### FTP-W-2

### SZB5 Active

FTP-AgSD can be used to treat burnwounds. The composition of SZB5, SZB5-2A, SZB5-3A, SB5, SB6, NP blend and other botanical blends are described in Example 5 below.

The Sangelose® Gel—90L—1% used in the formulations below is prepared in water containing 20% Aloe extract (Table 5):

TABLE 5A
FTP-W2-SZB5-1

| Ingredient | % w/w | Range |
|---|---|---|
| Chitosan (5%) | 35.14 | 38-39 |
| Petrolatum | 9.01 | 2-10 |
| PA4020V (20%) | 36.04 | 34-40 |
| Glycerin | 0.9 | 0.8-1 |
| Water | 7.11 | 5-15 |
| Arlacel | 0.9 | 0.001-1 |
| Tween 20 | 0.9 | 0.001-1 |
| SZB5-2A | 10 | 7-10 |
| Total | 100 | |

TABLE 5B
FTP-W2-SZB5-2

| Ingredient | % w/w |
|---|---|
| Petrolatum | 10 |
| Chitosan (5%) | 40 |
| Glycerine | 2 |
| Sorbitol mono laurate | 1 |
| PA4020V (50%) | 10 |
| Water | 27.23 |
| Tween 20 | 2 |
| SZB5-2A | 7.77 |
| Total | 100 |

TABLE 5C
FTP-W2-SZB5-3

| Ingredient | % w/w |
|---|---|
| Petrolatum | 5 |
| Chitosan (3%) | 50 |
| Sangelose ®-90L (1%) | 20 |
| Sorbitan oleate | 1 |
| Propylene glycol | 2 |
| PA4020V or PA-OCT | 10 |
| Water | 0 |
| Octanediol | 2 |
| SZB5-2A | 10 |
| Total | 100 |

TABLE 5D
FTP-W2-SZB5-4

| Ingredient | % w/w |
|---|---|
| Petrolatum | 5 |
| Chitosan (3%) | 40 |
| Sangelose ®-90L (1%) | 30 |
| Sorbitan oleate | 1 |
| Propylene glycol | 2 |
| PA4020V or PA-OCT | 10 |
| Water | 2 |
| SZB5-3 | 10 |
| Total | 100 |

TABLE 5E
FTP-W2-SZB5-5

| Ingredient | % w/w | Range |
|---|---|---|
| Petrolatum | 3.74 | 3-5 |
| Chitosan (3%) | 29.91 | 20-35 |
| Sangelose ®-90L (1%) | 17.76 | 15-25 |
| PA4020V or PA-OCT | 9.35 | 8-12 |
| Sorbitan oleate | 1.87 | 1-3 |
| Decanediol | 0.93 | 0.5-1.5 |
| Water | 26.17 | 22-30 |
| Tween 80 | 0.93 | 0.5-1.5 |
| SZB5 | 9.35 | 8-12 |
| Total | 100 | |

### BOT Active

TABLE 5F
FTP-W2-BS-1

| Ingredient | % w/w | Range |
|---|---|---|
| Petrolatum | 10 | 3-12 |
| Chitosan (5%) | 40 | 20-45 |
| Glycerin | 2 | 1-3 |
| Sorbitol mono laurate | 1 | 0.5-1.5 |
| PA4020V or PA-OCT (50%) | 10 | 5-20 |
| Water | 27 | 20-30 |
| Tween 20 | 2 | 0.5-3 |
| SB5 | 8 | 8-10 |
| Total | 100 | |

TABLE 5G
FTP-W2-BS-2

| Ingredient | % w/w |
|---|---|
| Petrolatum | 10 |
| Chitosan (5%) | 40 |
| Glycerin | 2 |
| Sorbitol mono laurate | 1 |
| PA4020V or PA-OCT (50%) | 10 |
| Water | 25 |
| Tween 20 | 2 |
| AgNO3 (0.5%) | 2 |
| SB5 | 8 |
| Total | 100 |

TABLE 5H
FTP-W2-BS-3

| Ingredient | % w/w |
|---|---|
| Petrolatum | 4.82 |
| Chitosan (5%) | 24.1 |
| Sangelose ®-90L (1%) | 24.1 |
| Tween 80 | 1.2 |
| PA4020V or PA-OCT (35%) | 34.94 |
| Decanediol | 1.2 |
| SB6 | 9.64 |
| Total | 100 |

TABLE 5I
FTP-W2-BS-4

| Ingredient | % w/w |
|---|---|
| Petrolatum | 3 |
| Chitosan (5%) | 20 |
| Sangelose ®-90L (1%) | 20 |
| PA4020V or PA-OCT (50%) | 20 |
| Sorbitan Oleate | 2.5 |
| Water | 25.5 |
| Decanediol | 1 |
| SB6 | 8 |
| Total | 100 |

Neosporin Active

TABLE 5J
FTP-W2-NP-1

| Ingredient | % w/w | Range |
|---|---|---|
| Petrolatum | 4.16 | 3-5 |
| Chitosan (3%) | 33.31 | 20-40 |
| Sangelose ®-90L (1%) | 16.66 | 10-25 |
| PA4020V or PA-OCT | 16.66 | 10-20 |
| NP Blend | 29.21 | 10-35 |
| Total | 100 | |

TABLE 5K
FTP-W2-NP-2

| Ingredient | % w/w | Range |
|---|---|---|
| Petrolatum | 4 | 3-5 |
| Chitosan (5%) | 20 | 12-25 |
| Sangelose ®-90L (1%) | 20 | 15-25 |
| Tween 80 | 1 | 0.5-1.5 |
| PA-OCT (53%) | 18.93 | 15-25 |
| Decanediol | 1 | 0.5-1.5 |
| NP Blend | 35.07 | 30-40 |
| Total | 100 | |

### Example 4

Various FTP-W3 compositions using alcohol as adescribed below were prepared (Table 6):

### FTP-W3

SZB5 Active

TABLE 6A
FTP-W3-SZB5-1

| Ingredient | % w/w | Range |
|---|---|---|
| Petrolatum | 4 | 3-5 |
| Chitosan (5%) | 10 | 5-30 |
| Sangelose ®-90L (1%) | 24.5 | 10-30 |
| PA-OCT-80 | 10 | 5-15 |
| Tween 80 | 2.5 | 1-3 |
| Ethanol 200 proof | 20 | 10-20 |
| Ethanol 200 proof + 10N NaOH | 6 | 5-10 |
| Decandiol | 1 | 1-2 |
| SZB5 | 10 | 5-10 |
| Glycerine | 8 | 5-10 |
| Almond oil | 4 | 2-5 |
| Total | 100 | |

TABLE 6B
FTP-W4-SZB5-1

| Ingredient | % w/w |
|---|---|
| Petrolatum | 4 |
| Chitosangel (5%) | 10 |
| Sangelose-90L gel(1%) | 24.5 |
| PA-OCT-80 | 10 |
| Tween 80 | 2.5 |
| Ethanol 200 proof | 20 |
| Ethanol 200 proof + 10N NaOH | 6 |
| Decanediol | 1 |
| SZB5-3 | 10 |
| Kytomer (7%) | 10 |
| Almond oil | 2 |
| Total | 100 |

Neosporin® Active

TABLE 6C
FTP-W4-NP-1

| Ingredient | % w/w | Range |
|---|---|---|
| Petrolatum | 3 | 3-5 |
| Chitosan (5%) | 20 | 10-40 |
| Sangelose ®-90L (1%) | 24.5 | 10-30 |
| PA-OCT-80 | 10 | 5-10 |
| Tween 80 | 2.5 | 1-3 |
| Alcohol (SDA-40B) + 10N | 4 | 3-10 |
| NaOH | | |
| Decandiol | 1 | 1-2 |
| NP Blend | 35 | 10-35 |
| Total | 100 | |

### Example 5

Film forming compositions for wounds without polyacetal polymer were prepared (FDP-W composition). The following FDP-W bases were prepared (Table 7).

Silver Sulfadiazine Based

TABLE 7A
FDP-W1-AgSD-1

| Ingredient | % w/w | Range |
|---|---|---|
| Petrolatum | 10 | 5-10 |
| Chitosan (5%) | 40 | 20-50 |
| Glycerin | 2 | 1-5 |
| Sorbitol mono laurate | 1 | 1-3 |
| AgSD | 1 | 1 |
| Water | 46 | 30-50 |
| Total | 100 | |

TABLE 7B
FDP-W1-SZB5-1

| Ingredient | % w/w | |
|---|---|---|
| Petrolatum | 5 | 3-5 |
| Chitosan (3%) | 62 | 30-70 |
| Sangelose ®-90L (1%) | 20 | 10-30 |
| Sorbitan oleate | 1 | 1-5 |
| Propylene glycol | 2 | 1-3 |
| SZB5-3 | 10 | 5-10 |
| Total | 100 | |

Botanical active Based

TABLE 7C
FDP-W1-BS-1

| Ingredient | % w/w | Range |
|---|---|---|
| Petrolatum | 83.33 | 50-90 |
| SB4 or SB5 | 7.41 | 5-10 |
| Oatmeal | 9.26 | 5-10 |
| Total | 100 | |

TABLE 7D
FDP-W1-BS-2

| Ingredient | % w/w | Range |
|---|---|---|
| Petrolatum | 82.87 | 50-85 |
| SB4 or SB5 | 7.37 | 5-10 |
| AgNO3 (0.5%) | 0.55 | 0.3-0.6 |
| Oatmeal | 9.21 | 5-10 |
| Total | 100 | |

TABLE 7E
FDP-W1-BS-3

| Ingredient | % w/w | Range |
|---|---|---|
| Petrolatum | 12 | 5-12 |
| Kytamer (5%) | 80 | 40-80 |
| SB4 or SB5 | 8 | 5-10 |
| Total | 100 | |

TABLE 7F
FDP-W1-BS-4

| Ingredient | % w/w | Range |
|---|---|---|
| Petrolatum | 11.93 | 10-50 |
| Kytamer (5%) | 79.52 | 40-90 |
| AgNO3 (0.5%) | 0.6 | 0-1 or 0.01-1 |
| SB4 or SB5 | 7.95 | 5-10 |
| Total | 100 | |

TABLE 7G
FDP-W1-BS-5

| Ingredient | % w/w |
|---|---|
| Alcohol (SDA-40B) | 64.34 |
| C-B-2-F Blend | 2.29 |
| Methocel (5%) | 3.93 |
| Water | 29.44 |
| Total | 100 |

TABLE 7H
FDP-W1-BS-6

| Ingredient | % w/w |
|---|---|
| Petrolatum | 9.80 |
| Chitosan (5%) | 39.22 |
| Aloe oil | 1.96 |
| Sangelose ®-90L (1%) | 39.22 |
| AgNO3 (0.5%) or | 1.96 |
| Ag2CO3 (0.5%) | |
| SB5 | 7.84 |
| Total | 100 |

TABLE 7I
FDP-W1-BS-7

| Ingredient | % w/w |
|---|---|
| Petrolatum | 3 |
| Chitosan (5%) | 20 |
| Sangelose ®-90L (1%) | 20 |
| Methocel (5%) | 10 |
| Sorbitan Oleate | 2.5 |
| Water | 35.5 |
| Decanediol | 1 |
| SB6 | 8 |
| Total | 100 |

Neosporin® Based

TABLE 7J
WC-SB-B7

| Ingredient | % w/w | Range |
|---|---|---|
| Petrolatum | 10 | 3-5 |
| Chitosan (5%) | 40 | 20-40 |
| Glycerin | 2 | 1-5 |
| Sorbitol mono laurate | 1 | 1-3 |
| NP Blend | 35.07 | 5-35 |
| Water | 12.93 | 5-20 |
| Total | 100 | |

BOT-L Based

TABLE 7K
WC-BOT-L-2

| Ingredient | % w/w | Range(% w/w) |
|---|---|---|
| Petrolatum | 4.4 | 3-5 |
| Chitosan (5%) | 29.5 | 20-30 |
| Sangelose ® (1%) | 29.5 | 20-30 |
| Sorbitan oleate | 3.7 | 2-4 |
| Water | 26.6 | 10-30 |
| Decanediol | 2.5 | 1-2 |
| AgNO3 (0.5%) | 0.6 | 0.3-0.6 |
| Zinc oxide | 0.1 | 0.1-0.2 |
| BOT-L-1 Blend | 3.1 | 3-5 |
| Total | 100 | |

TABLE 7L
WC-BOT-L2-1

| Ingredient | % w/w |
|---|---|
| Petrolatum | 4 |
| Chitosan (7%) | 57.85 |
| Sangelose ®-90L (1%) | 20 |
| Tween 80 | 1 |
| Decanediol | 1 |
| Colloidal Oats | 2 |
| Water | 9.15 |
| BOT-L-2 Blend | 5 |
| Total | 100 |

### Example 6

### Method of Preparing FTP Composition

The FTP compositions are prepared using addition of constituent parts sequentially to form a blend. First, Petrolatum is melted at 40° C. and Tween 80 is added and mixed well.

1% 90L Sangelose® gel in water containing 20% Aloe extract is added to the petrolatum, Tween 80 mixture and mixed well. After complete mixing, Decanediol is added and allowed to mix until dissolved. Ethanol (200 proof) is added and mixed well. Next, PA-OCT-80 is added to the stirring mixture and allowed to completely mix. Active ingredient (SZB, SB or NP) is added and stirred. A solution of 1% 10 N NaOH dissolved in ethanol 200 proof prepared in a separate container. The designated amount is added to the mixing solution (this amount varies depending on the wound cream being prepared). Chitosan gel is added drop-wise to the mixing solution.

For BOT wound cream, water is added to complete the formulation. For AgSD and NP, caprylic/capryl-triglyceride and glycerin is added as emollients. The pH is adjusted to 7.0 using NaOH.

### Example 7

Various antimicrobials were prepared as a blend containing emollients and wound healing agents.

Composition of various antimicrobial blends are listed below

1. Silversulfadiazine containing blends (AgSD blend)

2. Neosporin blends (NP blend)

3. Botanical antimicrobial containing blends (BOT blend)

All these antimicrobial blends can be added to FTP-W and FDP-W formulations (Table 8).

SZB5 Blends

TABLE 8A
SZB5-1

| Ingredient | % w/w |
|---|---|
| Silver sulfadiazine | 1 |
| Aloe gel | 0.12 |
| Zinc oxide | 0.3 |
| Calendula oil | 0.5 |
| Rosemary oil | 0.1 |
| Oatmeal | 0.5 |
| Almond oil | 0.5 |
| Zemea ® (propanediol) | 5 |
| CHG (20%) | 0.25 |
| Total | 8.27 |

TABLE 8B
SZB5-2A

| Ingredient | % w/w |
|---|---|
| Silver sulfadiazine | 1 |
| Aloe gel | 0.12 |
| Zinc oxide | 0.3 |
| Calendula oil | 0.5 |
| Rosemary oil | 0.1 |
| Almond oil | 0.5 |
| Zemea ® | 5 |
| CHG (20%) | 0.25 |
| Total | 7.77 |

TABLE 8C
SZB5-2B

| Ingredient | % w/w |
|---|---|
| Silver sulfadiazine | 1 |
| Aloe gel | 0.12 |
| Calendula oil | 0.53 |
| Rosemary oil | 0.1 |
| Glycerine | 1 |
| Zemea ® (propanediol) | 2 |
| CHG (20%) | 0.25 |
| Total | 5 |

TABLE 8D
SZB5-2C

| Ingredient | % w/w |
|---|---|
| Silver sulfadiazine | 1 |
| Aloe gel | 0.12 |
| Zinc oxide | 0.3 |
| Calendula oil | 0.5 |
| Rosemary oil | 0.1 |
| Almond oil | 0.75 |
| Zemea ® | 5 |
| CHG (20%) | 0.25 |
| Total | 8.02 |

TABLE 8E
SZB5-3

| Ingredient | % w/w |
|---|---|
| Silver sulfadiazine | 1 |
| Aloe gel | 0.12 |
| Calendula oil | 0.1 |
| Rosemary oil | 0.1 |
| Almond oil | 3.43 |
| Zemea ® (propanediol) | 5 |
| CHG (20%) | 0.25 |
| Total | 10 |

Botanical Active

TABLE 8F
SB3

| Ingredient | % w/w |
|---|---|
| Zemea ® (propanediol) | 2.9 |
| Flaxseed Oil | 2.5 |
| Calendular Extract | 1 |
| Aloe Powder | 0.1 |
| Benzyl Alcohol | 0.5 |
| Thymol | 0.1 |
| Basil | 0.1 |
| Rosemary oil | 0.1 |
| Lemon oil | 0.05 |
| Pomegranate Extract | 0.3 |
| Vit E | 0.1 |
| CHG (20%) | 0.25 |
| Total | 8 |

TABLE 8G
SB4

| Ingredient | % w/w |
|---|---|
| Zemea ® (propanediol) | 4.8 |
| Calendular Extract | 1 |
| Aloe Powder | 0.1 |
| Benzyl Alcohol | 0.5 |
| Benzoic Acid | 0.1 |
| Thymol | 0.1 |
| Basil | 0.1 |
| Rosemary oil | 0.1 |
| Lemon oil | 0.05 |
| Grapefruit seed extract | 0.8 |
| Vit E | 0.1 |
| CHG (20%) | 0.25 |
| Total | 8 |

TABLE 8H
SB5

| Ingredient | % w/w |
|---|---|
| Zemea ® (propanediol) | 4.75 |
| Calendular Extract | 1 |
| Aloe Powder | 0.1 |
| Benzyl Alcohol | 0.5 |
| Benzoic Acid | 0.1 |
| Thymol | 0.1 |
| Basil | 0.1 |
| Rosemary oil | 0.1 |
| Lemon oil | 0.05 |
| Orange oil | 0.05 |
| Grapefruit seed extract | 0.8 |
| Vit E | 0.1 |
| CHG (20%) | 0.25 |
| Total | 8 |

TABLE 8I
SB6

| Ingredient | % w/w |
|---|---|
| Zemea ® (propanediol) | 4.75 |
| Calendular Extract | 1 |
| Aloe Powder | 0.1 |
| Benzyl Alcohol | 0.5 |
| Benzoic Acid | 0.1 |
| Thymol | 0.1 |
| Basil | 0.1 |
| Rosemary oil | 0.1 |
| Lemon oil | 0.05 |
| Orange oil | 0.05 |
| Grapefruit seed extract | 0.8 |
| Vit E | 0.1 |
| Decanediol | 1 |
| CHG (20%) | 0.25 |
| Total | 9 |

TABLE 8J
C-B-1

| Ingredient | % w/w |
|---|---|
| Zemea ® (propanediol) | 2.5 |
| Flaxseed Oil | 2.5 |
| Calendular Oil | 0.5 |
| Aloe Powder | 0.1 |
| Thymol | 0.1 |
| Basil | 0.1 |
| Rosemary oil | 0.1 |
| Lemon Extract | 0.3 |
| Pomegranate Extract | 0.3 |
| Lactic Acid | 0.1 |
| Curcumin | 0.05 |
| Oatmeal | 0.6 |
| CHG (20%) | 0.25 |
| Total | 7.5 |

TABLE 8K
C-B-1-F

| Ingredient | % w/w |
|---|---|
| Phenoxyethanol | 1 |
| Octanediol | 0.5 |
| Thymol | 0.1 |
| Basil oil | 0.1 |
| Lemon Extract | 0.2 |
| Orange oil | 0.2 |
| Total | 2.1 |

TABLE 8L
C-B-2-F

| Ingredient | % w/w |
|---|---|
| Phenoxyethanol | 1 |
| Octanediol | 0.5 |
| Thymol | 0.1 |
| Basil oil | 0.1 |
| Rosemary oil | 0.1 |
| Lemon Extract | 0.3 |
| Orange oil | 0.1 |
| Benzyl alcohol | 1 |
| Total | 3.2 |

TABLE 8M
FAB-1-F

| Ingredient | % w/w |
|---|---|
| Phenoxyethanol | 1 |
| Octanediol | 0.3 |
| Thymol | 0.1 |
| Curcumin | 0.02 |
| Lemon Extract | 0.5 |
| Orange oil | 0.5 |
| Benzyl alcohol | 1 |
| Citric acid (add later) | 1 |
| Total | 4.42 |

TABLE 8N
FAB-2-F

| Ingredient | % w/w |
|---|---|
| Phenoxyethanol | 1 |
| Octanediol | 0.5 |
| Thymol | 0.1 |
| Lemongrass oil | 0.3 |
| Lemon Extract | 0.5 |
| Orange oil | 0.1 |
| Benzyl alcohol | 1 |
| Total | 3.5 |

Neosporin® Active

TABLE 8O
NP Blend

| Ingredient | % w/w |
|---|---|
| Neomycyin solution (0.01%) | 35 |
| Bacitracin | 0.007 |
| Polymyxin B | 0.064 |
| Total | 35.071 |

TABLE 8P
NP-2 Blend

| Ingredient | % w/w | % |
|---|---|---|
| Neomycyin Trisulfate Salt | 0.003 | 0.003 |
| Bacitracin | 0.007 | 0.007 |
| Polymyxin B | 0.064 | 0.064 |
| Total | 0.074 | |

BOT-L Active

TABLE 8Q
BOT-L-1

| Ingredient | % w/w |
|---|---|
| Zemea ® (propanediol) | 2.329 |
| Phenylethanol | 0.3 |
| Thymol | 0.01 |
| Benzoic Acid | 0.05 |
| Curcumin | 0.001 |
| Basil oil | 0.01 |
| Calendula oil | 0.1 |
| Rosemary oil | 0.05 |
| Red Sandle Paste | 0.05 |
| Aloe powder | 0.1 |
| Vitamin E | 0.1 |
| Total | 3.1 |

TABLE 8R
BOT-L-2

| Ingredient | % w/w |
|---|---|
| Zemea ® (propanediol) | 2.329 |
| Phenylethanol | 0.3 |
| Thymol | 0.01 |
| Benzoic Acid | 0.05 |
| Curcumin | 0.001 |
| Basil oil | 0.01 |
| Calendula oil | 0.1 |
| Rosemary oil | 0.05 |
| Red Sandle Paste | 0.05 |
| Aloe powder | 0.1 |
| Vitamin E | 0.1 |
| Almond oil | 3 |
| Water | 4 |
| Total | 10.1 |

BOT-L2 Active

TABLE 8S
BOT-L2-1

| Ingredient | % w/w |
|---|---|
| Phenyl ethanol | 0.3 |
| Ethylhexyl glycerin (Sensiva) | 0.5 |
| Zemea ® (propanediol) | 1.72 |
| Fenugreek oil | 0.2 |
| Thymol | 0.02 |
| Peppermint oil | 0.02 |
| Lemongrass oil | 0.02 |
| Lavender oil | 0.02 |
| Basil oil | 0.05 |
| Grapefruit seed extract | 0.5 |
| Calendula infused in Sun | 0.1 |
| flower oil | |
| Calendula extract | 1 |
| Rosemary oil | 0.05 |
| Lactic acid | 0.1 |
| Benzoic acid | 0.05 |
| Red sandal extract | 0.05 |
| Aloe powder (200x) | 0.1 |
| Tocopherol | 0.1 |
| Ascorbic acid | 0.1 |
| Decanediol | 0.5 |
| Total | 5.5 |

### Example 8

The following formulations were found to be stable and effective. Therefore these were prepared and tested (Table 9).

TABLE 9A
FTP-W4-SZB5-1

| Ingredient | % w/w | Range |
|---|---|---|
| Petrolatum | 4 | 3-5 |
| Chitosan (5%) | 10 | 10-15 |
| Sangelose ®-90L (1%) | 29.7 | 20-30 |
| PA-OCT-80 | 10 | 10-15 |
| Tween 80 | 2.5 | 1-3 |
| Ethanol 200 proof | 20 | 10-20 |
| Ethanol 200 proof + 10N NaOH | 6 | 3-7 |
| Decandiol | 1 | 1-2 |
| SZB5 | 10 | 10 |
| Caprylic Capric-Trygyceride | 5 | 3-8 |
| Glycerol | 1.8 | 1-2 |
| Total | 100 | |

TABLE 9B
FTP-W4-BS-1

| Ingredient | % w/w | Range |
|---|---|---|
| Petrolatum | 3 | 3-5 |
| Chitosan (5%) | 20 | 10-15 |
| Sangelose ®-90L (1%) | 24.5 | 20-30 |
| PA-OCT-80 | 10 | 10-15 |
| Tween 80 | 2.5 | 1-3 |
| Ethanol 200 proof | 20 | 10-20 |
| Ethanol 200 proof + 1N NaOH | 1 | 1-3 |
| Decanediol | 1 | 1-2 |
| Water | 10 | 5-10 |
| SB6 | 8 | 3-8 |
| Total | 100 | |

TABLE 9C
FTP-WC-NP-2

| Ingredient | % w/w | Range |
|---|---|---|
| Petrolatum | 4 | 3-5 |
| Chitosan (5%) | 10 | 10-15 |
| Sangelose ®-90L (1%) | 29.7 | 20-30 |
| PA-OCT-80 | 10 | 10-15 |
| Tween 80 | 2.5 | 1-3 |
| Ethanol 200 Proof | 28.99 | 10-30 |
| Ethanol 200 Proof + 10N NaOH | 6 | 3-7 |
| Decanediol | 1 | 1-2 |
| NP-2 Blend in water | 1.0074 | 1.005-1.008 |
| Caprylic Capric-Trygyceride | 5 | 3-8 |
| Glycerol | 1.8 | 1-2 |
| Total | 100 | |

Result

Antimicrobial efficacy by Pig Skin data for final wound cream formulations (Table 9D). Test organism; S. aureus

TABLE 9D

| Contact | | Log reduction from |
|---|---|---|
| Time | Group | Control growth |
| 2 hours | FTP-W4-SZB5-1 | 2.533 |
| | Cream-AgSD | 1.829 |
| 2 hours | FTP-W4-BS-1 | 2.447 |
| | Cream-BOT | 0.843 |
| 2 hours | FTP-WC-NP-2 | 3.586 |
| | Cream-NP | 2.373 |

The conclusion is that FTP groups show higher efficacy than the creams.

These topical wound healing compositions can be used for humans as well as animals; in various embodiments, the compositions can be in the form of medical or veterinary compositions.

### Example 9

### Film Forming Double Polymer and Triple Composition for Coating Medical Devices (FTP-M)

The FTP formulation was modified to obtain an optimal antimicrobial composition for medical device coatings. Antimicrobial agents such as silver sulfadiazine, silver oxide, silver carbonate, chlorhexidine and its salts, nitrofurazone, povidone iodine, combination of minocycline and silversulfadiazine can be used in the FTP-M base polymer.

Table 10 shows exemplary FTP compositions for medical devices (FTP-M).

TABLE 10

| Ingredient | % |
|---|---|
| PA-OCT | 1 to 6 |
| Polyurethane | 4 to 7 |
| (Tecoflex ® 93A) | |
| Polyurethane | 0.5 to 2 |
| (Tecoflex ® 60D) | |
| Decanediol | 2 to 5 |
| Tetrahydrofuran (THF) | 75 to 95 |

A chlorhexidine-free antimicrobial coating composition was developed to coat medical devices. This composition comprises of 2 types of polyurethane (specifically, aliphatic polyether-based thermoplastic polyurethanes (TPUs), such as Tecoflex™ 93A and Tecoflex™ 60D, both available from Lubrizol Corp. (Wickliffe, Ohio) and a degradable polyacetal polymer, decanediol and silver salts (FTP-M-AgSD) for coating urinary catheters.

The surface of the devices coated with FTP was more lubricious and had prolonged activity as compared to FDP coated devices. That is, efficacy in reducing bacterial adherence was found to be more than 5 days in the FTP AgSD-2S group, compared to 2 days in FDP-AgSD-2S group (see Table 17).

### Example 10

### Additional FTP-Silver Salt Coating Compositions

Method: 65 to 75% of previously prepared FTP-M was blended with comparable amounts of silver salts. Finally, the blend further solubilized using additional THF to make the volume to 100%.

### Table 11: FTP-M Silver Sulfadiazine (FTP-M-AgSD)

TABLE 11

| Ingredient | % w/v |
|---|---|
| PA-OCT | 2 to 5 |
| Polyurethane | 2 to 5 |
| (Tecoflex ® 93A) | |
| Polyurethane | 0.5 to 1 |
| (Tecoflex ® 60D) | |
| Decanediol | 1 to 3 |
| Tetrahydrofuran (THF) | 55 to 65 |
| Silver sulfadiazine | 0.5 to 2 |
| Tetrahydrofuran | 25 to 35 |

### Table 12: FTP-M Silver Sulfadiazine+Curcumin (FTP-M-AgSD-CUR)

TABLE 12

| Ingredient | % w/v |
|---|---|
| PA-OCT | 2 to 5 |
| Polyurethane | 2 to 5 |
| (Tecoflex ® 93A) | |
| Polyurethane | 0.5 to 1 |
| (Tecoflex ® 60D) | |
| Decanediol | 1 to 3 |
| Tetrahydrofuran (THF) | 55 to 65 |
| Silver sulfadiazine | 0.5 to 2 |
| Curcumin (CUR) | 0.5 to 2 |
| Tetrahydrofuran | 25 to 35 |

### Table 13: FTP-M Silver Nitrate (FTP-M-AgNO₃)

TABLE 13

| Ingredient | % w/v |
|---|---|
| PA-OCT | 2 to 5 |
| Polyurethane | 2 to 5 |
| (Tecoflex ® 93A) | |
| Polyurethane | 0.5 to 1 |
| (Tecoflex ® 60D) | |
| Decanediol | 1 to 3 |
| Tetrahydrofuran (THF) | 55 to 65 |
| Silver nitrate | 0.2 to 1 |
| Ammonium hydroxide | 0.2 to 1 |
| Tetrahydrofuran | 25 to 35 |

Table 14: FTP-M Silver Oxide (FTP-M-Ag₂CO₃)

TABLE 14

| Ingredient | % w/v |
|---|---|
| PA-OCT | 2 to 5 |
| Polyurethane | 2 to 5 |
| (Tecoflex ® 93A) | |
| Polyurethane | 0.5 to 1 |
| (Tecoflex ® 60D) | |
| Decanediol | 1 to 3 |
| Tetrahydrofuran (THF) | 55 to 65 |
| Silver carbonate | 0.1 to 1 |
| Ammonium hydroxide | 0.2 to 1 |
| Tetrahydrofuran | 25 to 35 |

### Example 11

### Biofilm Resistant Urinary Catheter

Evaluation of FTP-M-AgSD and FTP-M-AgSD-Curcumin (FTP-M-AgSD-CUR) catheters.

It was observed that when curcumin was used to coat the catheter, it improved the lubricity of the catheter. Latex urinary catheters were coated with FTP-M-AgSD and FTP-M-AgSD-CUR solution by soaking for the catheter in respective dipping solution for 30 to 90 seconds. Both the inner and outer surfaces were coated by this method. The composition of the coating solution is given in Example 10 above.

Urinary catheters coated with this novel technology (FTP-AgSD-UC) were found to render the catheter surface highly lubricious and biofilm resistant. The addition of curcumin to the solution increases the lubricity even further. Therefore, FTP-AgSD-UC does not require a second coating as in the case of the market leading silver alloy hydrogel UC (Bactiguard® & Bardex IC®): reducing the cost significantly. Based on the in vitro results, FTP-AgSD-UC can reduce catheter related infection significantly greater than the currently available silver UCs.

FTP-M-AgSD-UC and FTP-M-AgSD-CUR Initial Evaluation

The results of preliminary in vitro evaluation of FTP-AgSD-UC and its activity compared with two other antibacterial catheters are presented below as a proof of concept of this technology.

Table 15: The number of days the catheter stays sterile (1-100 CFU/plate): a comparative study with several commercially available catheters vs. FTP-AgSD-UC.

TABLE 15
Days of Sterility (1-100 CFU/plate)

| | | | | FTP-M | |
|---|---|---|---|---|---|
| | | Bactiguard ® | Bardex- | | AgSD- |
| Organism | Control | BIP | IC ® | AgSD | CUR |
| P. aeruginosa | 0 | 0 | 2 | 9 | 9 |
| C. albicans | 0 | 0 | 1 | 9 | 8 |

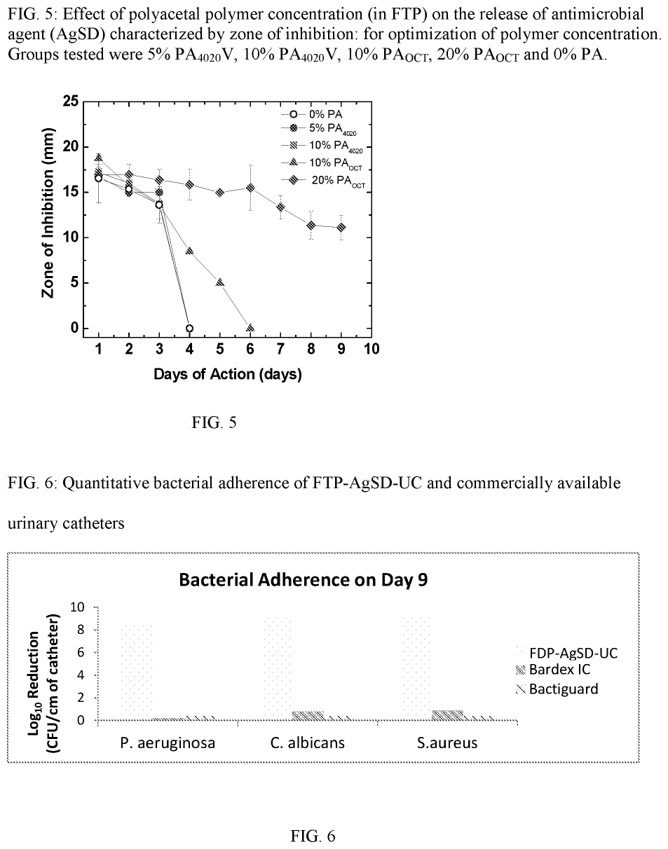

FIG. 6 shows quantitative bacterial adherence of FTP-AgSD-UC and commercially available urinary catheters.

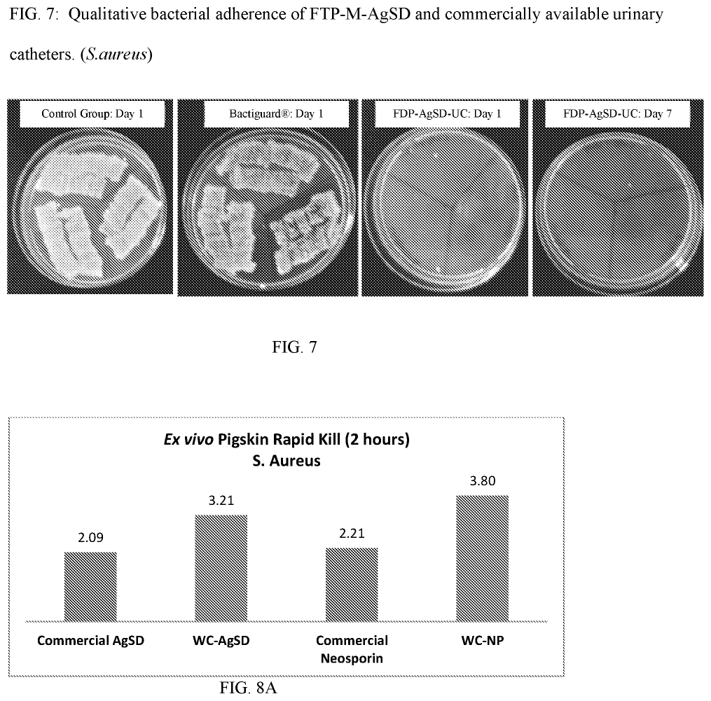

FIG. 7 shows qualitative bacterial adherence of FTP-M-AgSD and commercially available urinary catheters. (S. aureus).

Conclusion: FTP-AgSD-UC showed improved antimicrobial efficacy on Day 1 and retained this efficacy even at Day 7 of the bacterial challenge. In contrast, commercially available Bactiguard® and Bardex IC® (image not shown) did not show bacterial inhibition on Day 1 of the bacterial challenge. This clearly demonstrates the superiority of the present compositions in inhibiting bacterial growth.

### Example 12

Various FDP and FTP coating compositions were prepared Urinary catheters and central venous catheters were coated with these formulations and some of them were tested.

### FDP-Antimicrobial Coating Compositions

Polymer solution containing 4 gm. 93 A and gm 60 D Tecoflex® Polyurethane polymer in 60 mL THF (PU93A/60D) was prepared and used to prepare the following coating solutions:

FTP-AgSD-UC

FDP-AgSD-Sensiva 1 (FDP-Ag-S1)

| Ingredients | % w/w |
|---|---|
| Decanediol (DD) | 2 |
| Ethyl Hexyl glycerin (Sensiva ®) | 1 |
| Methanol | 10 |
| PU (93A/60D) | 65 |
| AgSD | 1 |
| THF | 21 |

FDP-AgSD-Sensiva-ZA-SS (FDP-Ag-S-ZA-SS)

| Ingredients | % w/w |
|---|---|
| Decanediol (DD) | 2 |
| Ethyl Hexyl glycerin | 1 |
| Zinc Acetate | 0.5 |
| Sodium Salicylate | 1 |
| Methanol | 10 |
| PU (93A/60D) | 65 |
| AgSD | 1 |
| THF | 19.5 |

FDP-AgSD-Sensiva-CU-SS (FDP-Ag-S-CU-SS)

| Ingredients | % w/w |
|---|---|
| Decanediol (DD) | 2 |
| Ethyl Hexyl glycerin | 1 |
| Copper sulfate | 0.2 |
| Sodium Salicylate | 0.5 |
| Methanol | 10 |
| PU (93A/60D) | 65 |
| AgSD | 1 |
| THF | 20.3 |

FDP-AgSD-Sensiva-CU-ZA-SS (FDP-Ag-S-CU-ZA-SS)

| Ingredients | % w/w |
|---|---|
| Decanediol (DD) | 2 |
| Ethyl Hexyl glycerin | 1 |
| Copper sulfate | 0.2 |
| Zinc Acetate | 0.2 |
| Sodium Salicylate | 0.5 |
| Methanol | 10 |
| PU (93A/60D) | 65 |
| AgSD | 1 |
| THF | 20.1 |

FDP-AgSD-Sensiva 2 (FDP-Ag-2S)

| Ingredients | % w/w |
|---|---|
| Decanediol (DD) | 2 |
| Ethyl Hexyl glycerin | 2 |
| Methanol | 10 |
| PU (93A/60D) | 65 |
| AgSD | 1 |
| THF | 20 |

FDP-AgSD-Sensiva 2-Zinc Salicylate (FDP-Ag-2S-ZS)

| Ingredients | % w/w |
|---|---|
| Decanediol (DD) | 2 |
| Ethyl Hexyl glycerin | 2 |
| Zinc Salicylate | 0.5 |
| Methanol | 10 |
| PU (93A/60D) | 65 |
| AgSD | 1 |
| THF | 19.5 |

FDP-AgSD-Sensiva 2-Nitrofurazone (FDP-Ag-2S-NF)

| Ingredients | % w/w |
|---|---|
| Decanediol (DD) | 2 |
| Ethyl Hexyl glycerin | 2 |
| Nitrofurazone | 1 |
| Methanol | 10 |
| PU (93A/60D) | 65 |
| AgSD | 1 |
| THF | 19 |

FDP-AgSD-Sensiva 2-N-Acetyl-L-Cysteine (FDP-Ag-2S-NAC)

| Ingredients | % w/w |
|---|---|
| Decanediol (DD) | 2 |
| Ethyl Hexyl glycerin | 2 |
| N-acetyl-L-cysteine | 1 |
| Methanol | 10 |
| PU (93A/60D) | 65 |
| AgSD | 1 |
| THF | 19 |

FDP-AgSD-Sensiva 2-Cranberry seed oil (FDP-Ag-2S-CBS)

| Ingredients | % w/w |
|---|---|
| Decanediol (DD) | 2 |
| Ethyl Hexyl glycerin | 2 |
| Cranberry seed oil | 1 |
| Methanol | 10 |
| PU (93A/60D) | 65 |
| AgSD | 1 |
| THF | 19 |

FDP-AgSD-Sensiva 2-Berberine (FDP-Ag-2S-BRB)

| Ingredients | % w/w |
|---|---|
| Decanediol (DD) | 2 |
| Ethyl Hexyl glycerin | 2 |
| Berberine | 1 |
| Methanol | 10 |
| PU (93A/60D) | 65 |
| AgSD | 1 |
| THF | 19 |

FTP-Antimicrobial Coating Compositions

FTP-AgSD-Sensiva 2-ZA-SS (FTP-Ag-2S-ZA-SS)

| Ingredients | % w/w |
|---|---|
| PA-OCT | 3 |
| Decanediol (DD) | 2 |
| Ethyl Hexyl glycerin | 1 |
| Zinc Acetate | 0.5 |
| Sodium Salicylate | 1 |
| Methanol | 10 |
| PU (93A/60D) | 65 |
| AgSD | 1 |
| THF | 16.5 |

FTP-AgSD-Sensiva-CU-ZA-SS (FDP-Ag-S-CU-ZA-SS)

| Ingredients | % w/w |
|---|---|
| PA-OCT | 3 |
| Decanediol (DD) | 2 |
| Ethyl Hexyl glycerin | 1 |
| Copper sulfate | 0.2 |
| Zinc Acetate | 0.2 |
| Sodium Salicylate | 0.5 |
| Methanol | 10 |
| PU (93A/60D) | 65 |
| AgSD | 1 |
| THF | 17.1 |

FTP-AgSD-Sensiva 2 (FTP-Ag-2S)

| Ingredients | % w/w |
|---|---|
| PA-OCT | 3 |
| Decanediol (DD) | 2 |
| Ethyl Hexyl glycerin | 2 |
| Methanol | 10 |
| PU (93A/60D) | 65 |
| AgSD | 1 |
| THF | 17 |

FTP-AgSD-Sensiva 2-Zinc Salicylate (FTP-Ag-2S-ZS)

| Ingredients | % w/w |
|---|---|
| PA-OCT | 3 |
| Decanediol (DD) | 2 |
| Ethyl Hexyl glycerin | 2 |
| Zinc Salicylate | 0.5 |
| Methanol | 10 |
| PU (93A/60D) | 65 |
| AgSD | 1 |
| THF | 16.5 |

FTP-AgSD-Sensiva 2-Nitrofurazone (FTP-Ag-2S-NF)

| Ingredients | % w/w |
|---|---|
| PA-OCT | 3 |
| Decanediol (DD) | 2 |
| Ethyl Hexyl glycerin | 2 |
| Nitrofurazone | 0.2 |
| Methanol | 10 |
| PU (93A/60D) | 65 |
| AgSD | 1 |
| THF | 16.8 |

FTP-AgSD-Sensiva 2-N-Acetyl-L-Cysteine (FTP-Ag-2S-NAC)

| Ingredients | % w/w |
|---|---|
| Decanediol (DD) | 2 |
| Ethyl Hexyl glycerin | 2 |
| N-acetyl-L-cysteine | 1 |
| Methanol | 10 |
| PU (93A/60D) | 65 |
| AgSD | 1 |
| THF | 19 |

FTP-AgSD-Sensiva 2-Cranberry seed oil (FDP-Ag-2S-CBS)

| Ingredients | % w/w |
|---|---|
| Decanediol (DD) | 2 |
| Sensiva | 2 |
| Cranberry seed oil | 1 |
| Methanol | 10 |
| PU (93A/60D) | 65 |
| AgSD | 1 |
| THF | 19 |

FTP-AgSD-Sensiva 2-Berberine (FDP-Ag-2S-BRB)

| Ingredients | % w/w |
|---|---|
| Decanediol (DD) | 2 |
| Sensiva | 2 |
| Berberine | 1 |
| Methanol | 10 |
| PU (93A/60D) | 65 |
| AgSD | 1 |
| THF | 19 |

Table 16: Adherence of E.Coli on catheters coated with FDP and FTP compositions

TABLE 16
Log₁₀ Reduction from control growth

| Group | Day 1 | Day 2 | Day 3 | Day 4 | Day 5 |
|---|---|---|---|---|---|
| FDP-AgSD | 3.0 | 3.0 | 1.50 | 0.24 | 0 |
| FTP AgSD | 5.75 | 5.74 | 2.94 | 0 | 0 |

Conclusion; FTP AgSD group is more effective inpreventing adherence than FDP-AgSD group

Table 17: Efficacy of catheters coated with FTP-AgSD composition containing various agents in preventing adherence.

TABLE 17A

| Group | Day 1 | Day 2 | Day 3 | Day 4 | Day 5 |
|---|---|---|---|---|---|
| FTP AgSD + 1Sensiva | 10 | 10 | 10 | 5.45 | 5.39 |
| FTP AgSD + 2Sensiva | 10 | 10 | 10 | 7.8 | 7.5 |
| FTP AgSD + Zinc | 10 | 6.9 | 3.47 | 0.5 | 0 |
| FTP AgSD + Cu | 10 | 6.58 | 3.48 | 0 | 0 |
| FDP AgSD + 2 sensiva | 7.5 | 6.9 | 0.18 | 0 | 0 |

Conclusion: Ethyl Hexyl Glycerin (sensiva) is more effective in enhancing the efficacy of FTP-AgSD than zinc acetate and copper sulfate. FTP AgSD+2Sensiva group show prolonged efficacy (more than 5 days) than FDP AgSD+2Sensiva group (2 days).

### Example 13

### Central Venous Catheters Coated with FDP and FTP

Central venous catheters were coated with the following coating compositions.

### FDP-AgSD-Sensiva 2 (FTP-Ag-2S)

### FDP-AgSD-Sensiva 2-Zinc Salicylate (FTP-Ag-2S-ZS)

### FDP-AgSD-Sensiva 2-Nitrofurazone (FTP-Ag-2S-NF)

### FDP-AgSD-Sensiva 2-N-Acetyl-L-Cysteine (FDP-Ag-2S-NAC)

FDP-AgSD-Sensiva 2-Cranberry seed oil (FDP-Ag-2S-CBS)

### FDP-AgSD-Sensiva 2-Berberine (FDP-Ag-2S-BRB)

### FTP-AgSD-Sensiva 2 (FTP-Ag-2S)

### FTP-AgSD-Sensiva 2-Zinc Salicylate (FTP-Ag-2S-ZS)

### FTP-AgSD-Sensiva 2-Nitrofurazone (FTP-Ag-2S-NF)

### FTP-AgSD-Sensiva 2-N-Acetyl-L-Cysteine (FDP-Ag-2S-NAC)

FDP-AgSD-Sensiva 2-Cranberry seed oil (FDP-Ag-2S-CBS)

### FTP-AgSD-Sensiva 2-Berberine (FDP-Ag-2S-BRB)

Conclusion: Among the Central venous catheters coated with the above groups, Ag-2S and Ag-2S-NAC of FDP and FTP groups were superior.

### Example 13A

### Synergistic Activity of CHX/AgSD with NAC

Conclusion; FTP AgSD group is more effective in preventing adherence than FDP-AgSD group

Table 17B: Rapid kill test: S. aureus (100 ul of 104 S. aureus culture)—15 second time kill

TABLE 17B

| Sample | |
|---|---|
| Control | Log₁₀ reduction |
| Chlorhexidine (CHX, 0.3%) | 4.51 |
| Silver silfadiazine (AgSD, 0.3%) | 3.07 |
| N-acetyl cystein (NAC, 1%) | 0.34 |
| CHX (0.3%) + NAC (1%) | 5.66 |
| AgSD (0.3%) + NAC (1%) | 3.5 |
| CHX (0.3%) + AgSD (0.3%) + NAC (1%) | 5.8 |

Conclusion: CHX (0.3%) showed antimicrobial efficacy synergistically with NAC (1%) against S. aureus.

Coating of Endotracheal tubes (ET) with FDP-Ag-2S-NAC solution.

Since this solution was effective in central venous catheters, endotracheal tubes were coated and tested with this solution.

Antimicrobial coating on Endotracheal Tube:

Endotracheal tube (PORTEX®, Smiths Medical, UK) was coated with chlorhexidine (1%, CHX) silver sulfadiazine (0.75%) and N-acetyl cystein (1%, NAC) either alone or in combination. Efficacy of the coated tubes was evaluated by zone of inhibition test against P. aeruginosa (ATCC 15442) after 24 hours.

Method: The anti-microbial efficacy of the coated endotracheal tube (ETT) was evaluated by zone of inhibition test against P. aeruginosa (ATCC 15442) according to the modified Kirby-Bauer method. Trypticase soy agar (TSA) plates were seeded with 0.3 mL of overnight grown bacterial culture diluted to 107 colony forming units (CFU) per mL.

Antimicrobial coated ETTs were cut into 0.5 cm segments and embedded vertically into the agar plate. Uncoated ETT segments were used as control. After 24 hours of incubation at 37° C., the diameters of zones of inhibition around the ETT segments, including the diameters of the ETT, were measured.

Table 17C: Zone of inhibition test against P. aeruginosa (ATCC 15442)

*ETT diameter (10 mm) is included.

TABLE 17C

| | Zone of inhibition against |
|---|---|
| Experimental groups | P. aeruginosa* (in mm scale) |
| Uncoated | 0 |
| CHX (1%) | 20 |
| NAC (1%) | 0 |
| AgSD (0.75%) | 0 |
| CHX (1%) + NAC (1%) | 24 |
| CHX (1%) + NAC (1%) + AgSD (0.75%) | 26 |

Conclusion:
ETT tubes coated with CHX (1%) + NAC (1%) + AgSD (0.75%) showed highest antimicrobial efficacy in zone of inhibition test against P. aeruginosa (ATCC 15442) after 24 hr

### Example 14

### FTP and FDP Polymer Complex for Use in Skin and Hand Disinfectants (FTP-D)

The compositions herein were found to have substantive activity in skin & hand disinfectants such as hand sanitizer, soap and the like. After application the disinfectant with substantive activity can continue to remain active against transient bacteria several minutes or hours post-application, thus reducing the risk of the spread of pathogens while caring for patients.

The following FTP-D and FDP-D polymer complexes were prepared:

Table 18: FTP-D

TABLE 18

| | Wt Range |
|---|---|
| Ingredient | in gms |
| Petrolatum | 0.1 to 1 |
| Chitosan (5%) | 2 to 10 |
| Sangelose ®-90L (1%) | 5 to 20 |
| Tween 80 | 0.1-1 |
| PA4020 or PA-OCT | 1-10 |

Table 18

TABLE 19

| | Wt Range |
|---|---|
| Ingredient | in gms |
| Petrolatum | 0.1 to 1 |
| Chitosan (5%) | 2 to 10 |
| Sangelose ®-90L (1%) | 5 to 20 |
| Tween 80 | 0.1-1 |
| Decanediol | 0.3-1 |

Table 20: Specific formula FTP-D1 (Mixture of the following ingredients) Total wt 13.4

TABLE 20

| Ingredient | Wt in gms |
|---|---|
| Petrolatum | 0.2 |
| Chitosan (5%) | 2 |
| Sangelose ®-90L (1%) | 10 |
| Tween 80 | 0.2 |
| PA-OCT | 1 |

Table 21: FDP-D-1 (Mixture of the following ingredients) Total wt 13.4 gins

TABLE 21

| Ingredient | Wt in gms |
|---|---|
| Petrolatum | 0.2 |
| Chitosan (5%) | 2 |
| Sangelose ®-90L (1%) | 11 |
| Tween 80 | 0.2 |

Table 22: General Formula for Hand sanitizer: FTP-DB and FDP-D-B

TABLE 22

| Ingredients | % w/w (Range) |
|---|---|
| SDA 3 C | 15-80 |
| FTP-D1/FDP-D1 | 10-20 |
| Zemea ® (propanediol) | 1-3 |
| Calendula Extract | 0.5-2 |
| Benzyl alcohol | 0.5-2 |
| Octanediol | 0-1 or 0.001 to 1 |
| Aloe extract | 1-3 |
| Lactic acid | 0.1-0.5 |
| Orange oil | 0.05-0.2 |
| Benzyl alcohol | 0.5-1 |
| Phenoxy ethanol | 0.5-1 |
| CHG (20%) | 0.25-2.5 |
| Water | 10-20 |

FTP disinfectants

Table 23: Alcohol Hand sanitizer—FTP-D-B

TABLE 23

| Ingredients | |
|---|---|
| Phase A | % w/w |
| SDA 3 C | 79.4 |
| FTP-D1 | 13.4 |
| Benzyl alcohol | 0.5 |
| Zemea ® (propanediol) ® | 1 |
| Lactic acid | 0.2 |
| Lemon oil | 0.1 |
| Phenoxyethanol | 0.7 |
| CHG(20%) | 1 |
| Ultrapure MFB 10 | 2 |
| Silicone 190 | 1 |
| cetrimonium chloride | 0.5 |
| Water | 0.2 |

Table 24: Aqueous Hand sanitizer—FTP-D-B

TABLE 24

| Ingredients | % w/w |
|---|---|
| Water | 61.4 |
| SDA3 C | 20 |
| FTP-D1 | 13.4 |
| Hydroxy ethyl cellulose | 0.2 |
| Benzyl alcohol | 0.5 |
| Octanediol | 0.5 |
| Zemea ® (propanediol) | 1 |
| Lactic acid | 0.2 |
| Lemon oil | 0.1 |
| Phenoxyethanol | 0.7 |
| CHG (20%) | 1 |
| Polyether-Modified Silicone | 0.5 |
| cetrimonium chloride | 1 |

Table 25: FTP-D-B foaming soap

TABLE 25

| Ingredients | % w/w |
|---|---|
| Water | 52.1 |
| Pluronic F 87 (nonionic surfactant) | 1 |
| Cocoamido propyl betaine | 9 |
| Dihydroxypropyl PEG-5 Linoleammonium Chloride | 2 |
| SDA 3 C | 17 |
| FTP-D1 | 13.4 |
| Benzyl alcohol | 1 |
| Octanediol | 0.5 |
| Zemea ® (propanediol) | 1 |
| Lactic acid | 0.2 |
| Lemon oil | 0.1 |
| Phenoxyethanol | 0.7 |
| CHG(20%) | 1 |
| cetrimonium chloride | 1 |

Table 26: Alcohol Hand sanitizer—FTP-D-B

TABLE 26

| Ingredients | |
|---|---|
| Phase A | % w/w |
| SDA 3 C | 79.4 |
| FDP-D1 | 13.4 |
| Benzyl alcohol | 0.5 |
| Zemea ® (propanediol) | 1 |
| Lactic acid | 0.2 |
| Lemon oil | 0.1 |
| Phenoxyethanol | 0.7 |
| CHG(20%) | 1 |
| Ultrapure MFB 10 | 2 |
| Silicone 190 | 1 |
| cetrimonium chloride | 0.5 |
| Water | 0.2 |

Table 24: Aqueous Hand sanitizer—FTP-D-B

TABLE 27

| Ingredients | % w/w |
|---|---|
| Water | 61.4 |
| SDA 3 C | 20 |
| FDP-D1 | 13.4 |
| Hydroxy ethyl cellulose | 0.2 |
| Benzyl alcohol | 0.5 |
| Octanediol | 0.5 |
| Zemea ® (propanediol) | 1 |
| Lactic acid | 0.2 |
| Lemon oil | 0.1 |
| Phenoxyethanol | 0.7 |
| CHG (20%) | 1 |
| Polyether-Modified Silicone | 0.5 |
| cetrimonium chloride | 0.5 |

Table 28: FTP-D-B foaming soap

TABLE 28

| Ingredients | % w/w |
|---|---|
| Water | 52.1 |
| Pluronic F 87 (nonionic surfactant) | 1 |
| Cocoamido propyl betaine | 9 |
| Dihydroxypropyl PFG-5 Linoleammonium Chloride | 2 |
| SDA 3 C | 17 |
| FDP-D1 | 13.4 |
| Benzyl alcohol | 1 |
| Octanediol | 0.5 |
| Zemea ® (propanediol) | 1 |
| Lactic acid | 0.2 |
| Lemon oil | 0.1 |
| Phenoxyethanol | 0.7 |
| CHG(20%) | 1 |
| cetrimonium chloride | 1 |

In various embodiments, FTP and FDP Soaps and sanitizers can also be prepared using any of the botanical blends listed in the present discolure, including but not limited to those denoted as SB2, SB3, SB4, SB5, SB6, CB1, CB1-F, CB2F, FAB 1 F or FAB 2F.

Table 29: FTP and FDP Surface Disinfectants

TABLE 29

| Ingredient | % w/w (range) |
|---|---|
| Water | 40-80 |
| FTP-D1/FDP-D1 | 10-20 |
| Isopropanol | 30-50 |
| phenoxyethanol | 7-10 |
| Benzyl alcohol | 5-10 |
| Lemon extract | 1-5 |
| Orange oil | 1-2 |
| Octanediol (Hydrolite-CG) | 1-5 |
| Zemea ® (propanediol) | 1-5 |
| Cocoamidopropyl betaine | 1-5 |
| (Dilute 5-10 fold with water before use) | |

### Example 15

### Additional Cream Composition (WC) Compositions were Evaluated In Vitro and Compared with Commercial Creams Containing Antibacterial Agents

Ranges that can be used in the following formulations

TABLE 30
WC-Ranges

| Ingredient | % w/w |
|---|---|
| Water | q.s to 100% |
| Isopropyl Myristate | 5-7 |
| Sorbitan Oleate | 1.5-3 |
| Stearyl Alcohol | 15-20 |
| Polyoxyl 40 Stearate | 5-8 |
| Flaxseed oil | 1-5 |
| Chlorhexidine gluconate (20%) | 0.01-0.5 |
| Propylene glycol | 3-6 |
| Petrolatum | 2-8 |
| Aloe gel 200X | 0.05-0.3 |
| Zinc oxide | 0.1-0.5 |
| Zinc gluconate | 0.1-0.5 |
| Zinc Salicylate | 0.1-0.5 |
| Calendula oil/extract | 0.1-1 or 1-3 |
| Rosemary oil | 0.01-0.3 |
| Almond oil | 1-5 |
| Ethylhexyl glycerin | 0.5-3 |
| Cocoa Butter | 2-5 |
| Chitosan (3%) | 3-8 |
| Grapefruit Seed Extract | 0.8 |
| Zemea ® | 1-10 |
| Thymol | 0.01-0.1 |
| Menthol | 0.01-0.1 |
| Tea tree oil | 0.1-0.5 |
| Orange oil | 0.1-0.5 |
| Benzyl alcohol | 0.1-2 |
| Calendula oil/extract | 0.5/2 |
| Cinnamon bark oil | 0.01-0.2 |
| Basil oil | 0.1-0.5 |
| Pomegranate oil | 0.1-1 |
| Phenoxyethanol | 0.1-1 |
| Octanediol | 0.1-1 |
| tetrahydrocurcumin (THC) | 0.01-1 |
| Red sandal wood paste | 0.01-1 |
| Lactic acid | 0.1-1 |
| Witch Hazel | 0.5-2 |

TABLE 31
WC-Base

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.05 |
| Propylene glycol | 5 |
| Petrolatum | 5.7 |
| Aloe gel 200X | 0.125 |
| Zinc oxide | 0.3 |
| Zinc gluconate | 0.1 |
| Calendula oil/extract | 0.5/2 |
| Rosemary oil | 0.1 |
| Chitosan (3%) | 5 |

TABLE 32
WC-AgSD-1

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.05 |
| Propylene glycol | 5 |
| Petrolatum | 5.7 |
| Aloe gel 200X | 0.125 |
| Zinc oxide | 0.3 |
| Zinc gluconate | 0.1 |
| Calendula oil/extract | 0.5/2 |
| Rosemary oil | 0.1 |
| Silver sulfadiazine | 1 |
| Almond oil | 2.5 |
| Ethylhexyl glycerin | 1 |
| Cocoa Butter | 4.1 |
| Chitosan (3%) | 5 |

TABLE 33
WC-AgSD-2

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.05 |
| Propylene glycol | 5 |
| Petrolatum | 5.7 |
| Aloe gel 200X | 0.125 |
| Zinc oxide | 0.3 |
| Zinc Salicylate | 0.1 |
| Calendula oil/extract | 0.5/2 |
| Rosemary oil | 0.1 |
| Silver sulfadiazine | 1 |
| Almond oil | 2.5 |
| Ethylhexyl glycerin | 1 |
| Cocoa Butter | 4.1 |

TABLE 34
WC-NP-1

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.3 |
| Propylene glycol | 5 |
| Lactic Acid | 0.01 |
| Petrolatum | 5.7 |
| Aloe gel 200X | 0.125 |
| Zinc oxide | 0.3 |
| Zinc gluconate | 0.1 |
| Calendula oil/extract | 0.5/2 |
| Rosemary oil | 0.1 |
| Neomycin Trisulfate Salt | 0.35 |
| Bacitracin | 0.7 |
| Polymyxin B | 0.064 |
| Grapefruit Seed Extract | 0.8 |
| Zemea ® | 2 |
| Thymol | 0.05 |
| Menthol | 0.05 |
| Tea tree oil | 0.3 |
| Orange oil | 0.2 |
| Ethylhexyl glycerin | 0.4 |
| Benzyl alcohol | 0.5 |
| Chitosan (3%) | 5 |

TABLE 35
WC-NP-2

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.3 |
| Propylene glycol | 5 |
| Lactic Acid | 0.01 |
| Petrolatum | 5.7 |
| Aloe gel 200X | 0.125 |
| Zinc oxide | 0.3 |
| Zinc Salicylate | 0.1 |
| Calendula oil/extract | 0.5/2 |
| Rosemary oil | 0.1 |
| Neomycin Trisulfate Salt | 0.35 |
| Bacitracin | 0.7 |
| Polymyxin B | 0.064 |
| Grapefruit Seed Extract | 0.8 |
| Zemea ® | 2 |
| Thymol | 0.05 |
| Menthol | 0.05 |
| Tea tree oil | 0.3 |
| Orange oil | 0.2 |
| Ethylhexyl glycerin | 0.4 |
| Benzyl alcohol | 0.5 |

TABLE 36
WC-NP-3

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.3 |
| Propylene glycol | 5 |
| Lactic Acid | 0.01 |
| Petrolatum | 5.7 |
| Aloe gel 200X | 0.2 |
| Aloe gel 1X | 5.0 |
| Zinc gluconate | 0.1 |
| Zinc oxide | 0.3 |
| Calendula oil/100%extract | 0.5 |
| Rosemary oil | 0.1 |
| Neomycin Trisulfate Salt | 0.35 |
| Bacitracin | 0.7 |
| Polymyxin B | 0.064 |
| Zemea ® | 2 |
| Menthol | 0.05 |
| Tea tree oil | 0.3 |
| Basil oil | 0.2 |
| Ethylhexyl glycerin | 0.5 |

TABLE 37
WC-BOT-1

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.3 |
| Propylene glycol | 5 |
| Lactic Acid | 0.01 |
| Petrolatum | 5.7 |
| Aloe gel 200X | 0.125 |
| Zinc oxide | 0.3 |
| Zinc gluconate | 0.1 |
| Calendula oil/extract | 0.5/2 |
| Rosemary oil | 0.1 |
| Zemea ® | 5 |
| Thymol | 0.1 |
| Tea tree oil | 0.3 |
| Menthol | 0.1 |
| Cinnamon bark oil | 0.1 |
| Basil oil | 0.3 |
| Pomegranate oil | 0.5 |
| Benzyl alcohol | 1 |
| Phenoxyethanol | 0.7 |
| Octanediol | 0.5 |
| THC | 0.1 |
| Red sandal wood paste | 0.1 |
| Lactic acid | 0.5 |
| Witch Hazel | 1 |
| Grapefruit seed extract | 0.8 |
| Chitosan (3%) | 5 |

TABLE 38
WC-BOT-2

| Ingredient | % w/w |
|---|---|
| Water | q.s to 100% |
| Isopropyl Myristate | 5.7 |
| Sorbitan Oleate | 2.5 |
| Stearyl Alcohol | 18 |
| Polyoxyl 40 Stearate | 6.3 |
| Flaxseed oil | 3 |
| Chlorhexidine gluconate (20%) | 0.3 |
| Propylene glycol | 5 |
| Lactic Acid | 0.01 |
| Petrolatum | 5.7 |
| Aloe gel 200X | 0.125 |
| Zinc oxide | 0.3 |
| Zinc Salicylate | 0.1 |
| Calendula oil/extract | 0.5/2 |
| Rosemary oil | 0.1 |
| Zemea ® | 5 |
| Thymol | 0.1 |
| Tea tree oil | 0.3 |
| Menthol | 0.1 |
| Cinnamon bark oil | 0.1 |
| Basil oil | 0.3 |
| Pomegranate oil | 0.5 |
| Benzyl alcohol | 1 |
| Phenoxyethanol | 0.7 |
| Octanediol | 0.5 |
| THC | 0.1 |
| Red sandal wood paste | 0.1 |
| Lactic acid | 0.5 |
| Witch Hazel | 1 |
| Grapefruit seed extract | 0.8 |

### Example 16

### Additional FTP Compositions were Evaluated In Vitro and Compared with Commercial Creams Containing Antibacterial Agents

In the Tables below, BTMSCB Emulsifier is Behentrimonium Methosulfate (and) Cetyl Alcohol (and) Butylene Glycol.

TABLE 39
FTP-Ranges

| Ingredient | % w/w |
|---|---|
| Water | q.s to 100% |
| Flaxseed oil | 1-5 |
| Petrolatum | 2-8 |
| Sangelose (1%) | 10-40 |
| Tween 80 | 1-5 |
| Decanediol | 0.5-3 |
| Zinc oxide | 0.1-1 |
| Zinc salicylate | 0.1-1 |
| Ethanol 200 Proof | 5-20 |
| PA-OCT-80 | 2-20 |
| Chitosan | 0.1-1 |
| Silver sulfadiazine | 0.5-3 |
| Aloe Gel 200X | 0.05-0.5 |
| Calendula Extract | 0.1-1 |
| Rosemary oil | 0.05-5 |
| Zemea ® | 1-10 |
| Glycerin | 1-10 |
| Ethylhexylglycerin | 0.5-3 |
| Chlorhexidine gluconate (20%) | 0.01-0.5 |
| Caprylic Capric Triglyceride | 1-10 |
| BTMSCB Emulsifier | 0.5-5 |
| Polawax Emulsifier | 0.5-5 |
| Bacitracin | 0.1-1.5 |
| Polymyxin B | 0.01-0.5 |
| Neomycin Trisulfate Salt | 0.1-1 |
| Grapefruit Seed Extract | 0.5-2 |
| Zemea ® | 0.1-10 |
| Thymol | 0.01-0.5 |
| Menthol | 0.01-0.5 |
| Tea tree oil | 0.1-0.5 |
| Orange oil | 0.1-0.5 |
| Ethylhexyl glycerin | 0.1-5 |
| Benzyl alcohol | 0.1-2 |
| Red Sandalwood | 0.01-0.5 |
| Curcumin | 0.01-0.5 |

TABLE 40
FTP-AgSD

| Ingredient | % w/w |
|---|---|
| Water | q.s to 100% |
| Flaxseed oil | 3 |
| Petrolatum | 4 |
| Sangelose (1%) | 30 |
| Tween 80 | 2.5 |
| Decanediol | 1 |
| Zinc oxide | 0.3 |
| Zinc salicylate | 0.1 |
| Ethanol 200 Proof | 10 |
| PA-OCT-80 | 10 |
| Chitosan (3%) | 10 |
| Silver sulfadiazine | 1 |
| Aloe Gel 200X | 0.12 |
| Calendula Extract | 0.3 |
| Rosemary oil | 0.1 |
| Zemea ® | 3.62 |
| Glycerin | 3.61 |
| Ethylhexylglycerin | 1 |
| Chlorhexidine gluconate (20%) | 0.25 |
| Caprylic Capric Triglyceride | 5 |
| Glycerin | 1.5 |
| BTMSCB Emulsifier | 1.5 |
| Polawax Emulsifier | 1.5 |

TABLE 41
FTP-NP

| Ingredient | % w/w |
|---|---|
| Water | q.s to 100% |
| Flaxseed oil | 3 |
| Petrolatum | 4 |
| Sangelose (1%) | 30 |
| Tween 80 | 2.5 |
| Decanediol | 1 |
| Zinc oxide | 0.3 |
| Zinc salicylate | 0.1 |
| Ethanol 200 Proof | 10 |
| PA-OCT-80 | 10 |
| Chitosan (3%) | 10 |
| Neomycin Trisulfate Salt | 0.35 |
| Bacitracin | 0.7 |
| Polymyxin B | 0.064 |
| Grapefruit Seed Extract | 0.8 |
| Zemea ® | 2 |
| Thymol | 0.05 |
| Menthol | 0.05 |
| Tea tree oil | 0.3 |
| Orange oil | 0.2 |
| Ethylhexyl glycerin | 0.4 |
| Benzyl alcohol | 0.5 |
| Caprylic Capric Triglyceride | 5 |
| Glycerin | 1.5 |
| BTMSCB Emulsifier | 1.5 |
| Polawax Emulsifier | 1.5 |

TABLE 42
FTP-PVI

| Ingredient | % w/w |
|---|---|
| Water | q.s to 100% |
| Flaxseed oil | 3 |
| Petrolatum | 4 |
| Sangelose (1%) | 30 |
| Tween 80 | 2.5 |
| Decanediol | 1 |
| Zinc oxide | 0.3 |
| Zinc salicylate | 0.1 |
| Ethanol 200 Proof | 10 |
| PA-OCT-80 | 10 |
| Chitosan (3%) | 10 |
| Povidone-iodine | 5 |
| Grapefruit Seed Extract | 0.8 |
| Zemea ® | 2 |
| Thymol | 0.05 |
| Menthol | 0.05 |
| Tea tree oil | 0.3 |
| Orange oil | 0.2 |
| Ethylhexyl glycerin | 0.4 |
| Benzyl alcohol | 0.5 |
| Caprylic Capric Triglyceride | 5 |
| Glycerin | 1.5 |
| BTMSCB Emulsifier | 1.5 |
| Polawax Emulsifier | 1.5 |

### Example 17

### Preparation and Composition of Triple Polymer Solution for Coating Silicone Urinary and Central Venous Catheters

Method: The FTP-M-AgSD coating solutions are prepared by mixing all ingredients (except silver sulfadiazine) stepwise until a completely soluble solution is formed. Separately, a slurry of silver sulfadiazine is made using small amounts of THF and transferred directly to the soluble polymer solution to render an opaque slurry of the coating solution, FTP-M-AgSD.

Silicone urinary catheters or silicone central venous catheters were dipped in the solution for 1 minute, dried at room temperature for 24 to 48 hours.

FTP-M Silver Sulfadiazine (FTP-M-AgSD) Compositions

TABLE 43
FTP-M-Ranges

| Ingredient | % w/v |
|---|---|
| PA-OCT-80 | 2-5 |
| Polyurethane | 1-6 |
| (Tecoflex ® 93A) | |
| Polyurethane | 0.5-4 |
| (Tecoflex ® 60D) | |
| Decanediol | 0.5-5 |
| Tetrahydrofuran (THF) | 50-70 |
| Chlorhexidine | 0-5 or 0.001 to 5 |
| Silver salts | 0.5-1 |
| Zinc Salts | 0-2 |
| Medical Adhesives | 0-20 or 0.001 to 20 |
| (silicone & urethane) | |

TABLE 44
FTP-M-AgSD-1

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Tetrahydrofuran | q.s to 100% |

TABLE 45
FTP-M-AgSD-2

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 1 |
| Tetrahydrofuran (THF) | 61.1 |
| Silver sulfadiazine | 1 |
| Ethylhexyl Glycerin | 2 |
| Methanol | 10 |
| Tetrahydrofuran | q.s to 100% |

TABLE 46
FTP-M-AgSD-3

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Silicone Adhesive (A-100) | 3 |
| Tetrahydrofuran | q.s to 100% |

TABLE 47
FTP-M-AgSD-4

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Tetrahydrofuran | q.s to 100% |

TABLE 48
FTP-M-AgSD-5

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Loctite M-06FL | 5 |
| Urethane Adhesive | |
| Tetrahydrofuran | q.s to 100% |

TABLE 49
FTP-M-AgSD-Z

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Silicone Adhesive (A-100) | 3 |
| Zinc salicylate | 0.2 |
| Tetrahydrofuran | q.s to 100% |

TABLE 50
FTP-M-CHX-Z-1

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10. |
| Silicone Adhesive (A-100) | 3 |
| Zinc salicylate | 0.2 |
| Tetrahydrofuran | q.s to 100% |

TABLE 51
FTP-M-CHX-Z-2

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10 |
| Silicone Adhesive (A-100) | 3 |
| Tetrahydrofuran | q.s to 100% |

TABLE 52
FTP-M-CHX-Z-3

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10 |
| Silicone Adhesive (A-100) | 3 |
| Zinc salicylate | 0.1 |
| Zinc gluconate | 0.2 |
| Zinc lactate | 0.1 |
| Tetrahydrofuran | q.s to 100% |

TABLE 53
FTP-M-AgSD-Z-4

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Loctite M-06FL | 5 |
| Urethane Adhesive | |
| Zinc salicylate | 0.2 |
| Tetrahydrofuran | q.s to 100% |

The antimicrobials that can be used with this system include synthetic antimicrobial agents, including but not limited to: silver salts (e.g., silver sulfadiazine, silver nitrate, silver oxide, silver carbonate) chlorhexidine and its salts, benzalkonium chloride, povidone iodine, nitrofurazone, miconazole, Neosporin® (bacitracin, polymyxin, neomycin) in amounts of, for example, 0.005 to 5% w/w each. Botanical antimicrobial agents include, but are not limited to, essential oils and botanical extracts selected from orange oil, lemon oil, lemon grass oil, basil oil rosemary oil, thymol, cinnamon bark oil, tetrahydrocurcumin, lavender oil, lemon oil extract, grape fruit seed extract, pomegranate extract, benzyl alcohol, phenylethanol, in amounts of, for example, 0.05 to 1% w/w each.

Wound healing agents include but not limited to aloe powder, aloe extract, oat powder or meal, oat oil, oat beta glucan, calendula oil, zinc salts, in amounts of, for example, 0.2 to 5% w/w each.

Emollient solvents include, but are not limited to: alkanediol, phenoxyethanol or benzyl alcohol, in amounts of, for example, 0.3 to 2% w/w each.

### Example 18

### Biofilm Resistant Urinary Catheter

Silicone urinary and central venous catheters were coated with FTP-M-AgSD solution by soaking the catheter for 60 seconds. Both the inner and outer surfaces are coated for urinary catheters, while only the outside is coated for central venous catheters (by sealing at one end before coating). The composition of the coating solution is given above.

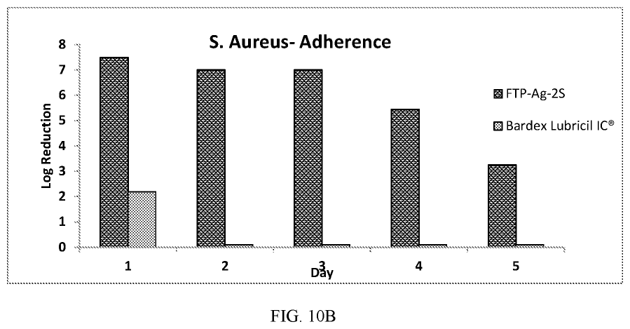

Urinary catheters coated with this novel technology (FTP-AgSD-UC) can render the catheter surface highly lubricious and biofilm resistant. Therefore, FTP-AgSD-UC does not require a second coating as in the case of the market leading silver alloy hydrogel UC (Bactiguard® & Bardex IC®): reducing the cost significantly. Based on in vitro results, FTP-AgSD-UC can reduce catheter related infection significantly greater than the currently available silver UCs. Results are shown in Table 54 and FIGS. 10A and 10B.

Table 54: Qualitative efficacy of silicone FTP-AgSD-UC vs. Bard Lubrisil IC®

TABLE 54

| | | | Subculture of | |
|---|---|---|---|---|
| | | | Solution | |
| Inoculum | Group | Day | (around catheter) | Adherence |
| E. Coli | FTP-AgSD-UC | Day 1 | 2+ | 0 |
| (10⁵ CFU/ml) | (silicone) | Day 2 | 4+ | 0 |
| | | Day 3 | 4+ | 0 |
| | | Day 4 | 4+ | 30 |
| | | Day 5 | 4+ | 40 |
| | | Day 6 | 4+ | 40 |
| | | Day 7 | 4+ | 40 |
| | | Day 8 | Heavy | Heavy |
| | Bard Lubrisil | Day 1 | Heavy | Heavy |
| | IC ® (silicone) | Day 2 | Heavy | Heavy |
| S. Aureus | FTP-AgSD-UC | Day 1 | 2+ | 0 |
| (10⁵ CFU/ml) | (silicone) | Day 2 | 4+ | 0 |
| | | Day 3 | 4+ | 0 |
| | | Day 4 | 4+ | 40 |
| | | Day 5 | 4+ | 60 |
| | | Day 5 | Heavy | Heavy |
| | Bard Lubrisil | Day 1 | Heavy | Heavy |
| | IC ® (silicone) | Day 2 | Heavy | Heavy |

### Example 19

### Preparation and Composition of Triple Polymer Solution for Coating Silicone Central Venous Catheters

TABLE 55
FTP-M-AgSD-S-CVC-1

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | |
| Tetrahydrofuran (THF) | 61.1 |
| Silver sulfadiazine | 1 |
| Tetrahydrofuran | q.s to 100% |

TABLE 56
FTP-M-AgSD-S-CVC-2

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 1 |
| Tetrahydrofuran (THF) | 61.1 |
| Silver sulfadiazine | 1 |
| Ethylhexyl Glycerin | 2 |
| Methanol | 10 |
| Tetrahydrofuran | q.s to 100% |

TABLE 57
FTP-M-AgSD-S-CVC-3

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Silicone Adhesive (A-100) | 3 |
| Tetrahydrofuran | q.s to 100% |

TABLE 58
FTP-M-CHX-S-CVC-4

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10 |
| Tetrahydrofuran | q.s to 100% |

TABLE 59
FTP-M-CHX-S-CVC-5

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10 |
| Zinc salicylate | 0.1 |
| Tetrahydrofuran | q.s to 100% |

TABLE 60
FTP-M-CHX-S-CVC-6

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10 |
| Zinc gluconate | 0.2 |
| Tetrahydrofuran | q.s to 100% |

TABLE 61
FTP-M-CHX-S-CVC-7

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | |
| Tetrahydrofuran (THF) | 61.1 |
| Chlorhexidine | 2 |
| Ethylhexyl Glycerin | 2 |
| Methanol | 10 |
| Zinc lactate | 0.1 |
| Tetrahydrofuran | q.s to 100% |

TABLE 62
FTP-M-CHX-S-CVC-8

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Chlorhexidine | 2 |
| Ethylhexyl Glycerin | 2 |
| Methanol | 10 |
| Zinc salicylate | 0.1 |
| Zinc gluconate | 0.2 |
| Zinc lactate | 0.1 |
| Tetrahydrofuran | q.s to 100% |

TABLE 63
FTP-M-AgSD-S-CVC-5

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Loctite M-06FL | 5 |
| Urethane Adhesive | |
| Tetrahydrofuran | q.s to 100% |

TABLE 64
FTP-M-AgSD-S-CVC-6

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Zinc salicylate | 0.1 |
| Loctite M-06FL | 5 |
| Urethane Adhesive | |
| Tetrahydrofuran | q.s to 100% |

TABLE 65
FTP-M-AgSD-S-CVC-7

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Zinc gluconate | 0.2 |
| Loctite M-06FL | 5 |
| Urethane Adhesive | |
| Tetrahydrofuran | q.s to 100% |

TABLE 66
FTP-M-AgSD-S-CVC-8

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Zinc lactate | 0.1 |
| Loctite M-06FL | 5 |
| Urethane Adhesive | |
| Tetrahydrofuran | q.s to 100% |

TABLE 67
FTP-M-AgSD-S-CVC-9

| Ingredient | % w/v |
|---|---|
| PA-OCT-80 | 3 |
| Polyurethane | 3.1 |
| (Tecoflex ® 93A) | |
| Polyurethane | 0.8 |
| (Tecoflex ® 60D) | |
| Decanediol | 2 |
| Tetrahydrofuran (THF) | 61.1 |
| Silver sulfadiazine | 1 |
| Ethylhexyl Glycerin | 2 |
| Methanol | 10 |
| Zinc salicylate | 0.1 |
| Zinc gluconate | 0.2 |
| Zinc lactate | 0.1 |
| Loctite M-06FL | 5 |
| Urethane Adhesive | |
| Tetrahydrofuran | q.s to 100% |

In certain embodiments, the FTP-M-AgSD-S-CVC coating solutions are prepared by mixing all ingredients (except silver sulfadiazine) stepwise until a completely soluble solution is formed. Separately, a slurry of silver sulfadiazine is made using small amounts of THF and transferred directly to the soluble polymer solution to render an opaque slurry of the coating solution, FTP-M-AgSD-S-CVC.

Silicone Central Venous Catheters were sealed on both ends (to prevent inner lumen coating) and dipped in the solution for 5 to 60 seconds, then removed from contact and dried at room temperature for 24 to 48 hours. In various embodiments, this drying step could be done for 24 to 30 hours, 24 to 36 hours, 30 to 36 hours, 32 to 40 hours, 32 to 44 hours or 30 to 48 hours.

### Example 20

### Preparation and Composition of Triple Polymer Solution for Coating Polyurethane Central Venous Catheters

TABLE 68
FTP-M-AgSD-PU-CVC-Range

| Ingredient | % w/v |
|---|---|
| PA-OCT-80 | 1-5 |
| Polyurethane | 2-5 |
| (Tecoflex ® 93A) | |
| Polyurethane | 1-5 |
| (Tecoflex ® 60D) | |
| Decanediol | 1-5 |
| Tetrahydrofuran (THF) | 50-70 |
| Silver sulfadiazine | 0.5-2 |
| Methanol | 5-25 |
| Ethylhexyl Glycerin | 0.5-3 |
| Silicone Adhesive | 0.5-3 |
| Dow Corning MD7-4502 | |
| Loctite M-06FL | 0.1-15 |
| Urethane Adhesive | |
| Zinc salts | 0.1-2 |
| Tetrahydrofuran | q.s to 100% |

TABLE 69
FTP-M-AgSD-PU-CVC-1

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Silicone Adhesive | 1 |
| Dow Corning MD7-4502 | |
| Tetrahydrofuran | q.s to 100% |

TABLE 70
FTP-M-AgSD-PU-CVC-2

| Ingredient | % w/v |
|---|---|
| PA-OCT-80 | 3. |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Silicone Adhesive | 1 |
| Dow Corning MD7-4502 | |
| Loctite M-06FL | 5 |
| Urethan Adhesive | |
| Tetrahydrofuran | q.s to 100% |

TABLE 71
FTP-M-AgSD-PU-CVC-3

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Zinc salicylate | 0.1 |
| Silicone Adhesive | 1 |
| Dow Corning MD7-4502 | |
| Loctite M-06FL | 5 |
| Urethane Adhesive | |
| Tetrahydrofuran | q.s to 100% |

TABLE 72
FTP-M-AgSD-PU-CVC-4

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Zinc gluconate | 0.2 |
| Silicone Adhesive | 1 |
| Dow Corning MD7-4502 | |
| Loctite M-06FL | 5 |
| Urethane Adhesive | |
| Tetrahydrofuran | q.s to 100% |

TABLE 73
FTP-M-AgSD-PU-CVC-5

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Zinc lactate | 0.1 |
| Silicone Adhesive | 1 |
| Dow Corning MD7-4502 | |
| Loctite M-06FL | 5 |
| Urethane Adhesive | |
| Tetrahydrofuran | q.s to 100% |

TABLE 74
FTP-M-AgSD-PU-CVC-6

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Zinc salicylate | 0.1 |
| Zinc gluconate | 0.2 |
| Zinc lactate | 0.1 |
| Silicone Adhesive | 1 |
| Dow Corning MD7-4502 | |
| Loctite M-06FL | 5 |
| Urethane Adhesive | |
| Tetrahydrofuran | q.s to 100% |

TABLE 75
FTP-M-CHX-PU-CVC-3

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 2 |
| Methanol | 16 |
| Ethylhexyl Glycerin | 2 |
| Loctite M-06FL | 5 |
| Urethane Adhesive | |
| Tetrahydrofuran | q.s to 100% |

TABLE 76
FTP-M-CHX-PU-CVC-4

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 2 |
| Methanol | 16 |
| Ethylhexyl Glycerin | 2 |
| Zinc salicylate | 0.1 |
| Loctite M-06FL | 5 |
| Urethane Adhesive | |
| Tetrahydrofuran | q.s to 100% |

TABLE 77
FTP-M-CHX-PU-CVC-5

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 2 |
| Methanol | 16 |
| Ethylhexyl Glycerin | 2 |
| Zinc gluconate | 0.2 |
| Loctite M-06FL | 5 |
| Urethane Adhesive | |
| Tetrahydrofuran | q.s to 100% |

TABLE 78
FTP-M-CHX-PU-CVC-6

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 2 |
| Methanol | 16 |
| Ethylhexyl Glycerin | 2 |
| Zinc lactate | 0.1 |
| Loctite M-06FL | 5 |
| Urethane Adhesive | |
| Tetrahydrofuran | q.s to 100% |

TABLE 79
FTP-M-CHX-PU-CVC-7

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 2 |
| Methanol | 16 |
| Ethylhexyl Glycerin | 2 |
| Zinc salicylate | 0.1 |
| Zinc gluconate | 0.2 |
| Zinc lactate | 0.1 |
| Loctite M-06FL | 5 |
| Urethane Adhesive | |
| Tetrahydrofuran | q.s to 100% |

Coating Method: The FTP-M-AgSD-PU-CVC coating solutions are prepared by mixing all ingredients (except silver sulfadiazine) step wise until a completely soluble solution is formed. Separately, a slurry of silver sulfadiazine is made using small amounts of THF and transferred directly to the soluble polymer solution to render an opaque slurry of the coating solution, FTP-M-AgSD-PU-CVC.

Polyurethane central venous catheters were sealed on both ends (to prevent inner lumen coating) and dipped in the solution for 5 seconds, dried at room temperature for 24-48 hours.

### Example 21

### Tables Showing Qualitative Efficacy of Different Coated Catheters

Table 80 shows qualitative efficacy of FTP-M-AgSD-PU-CVC-1.

TABLE 80

| | | | Subculture of | Adherence |
|---|---|---|---|---|
| | | | Solution | #colony/cm |
| Inoculum | Group | Day | (around catheter) | catheter |
| E. Coli | FTP-M- | Day 1 | 2+ | 0 |
| (10⁵ CFU/ml) | AgSD- | Day 2 | 4+ | 0 |
| | PU-CVC-1 | Day 3 | 4+ | 0 |
| | | Day 4 | 4+ | 40 |
| | | Day 5 | 4+ | 50 |
| | | Day 6 | Heavy | Heavy |
| S. Aureus | FTP-M- | Day 1 | 2+ | 0 |
| (10⁵ CFU/ml) | AgSD- | Day 2 | 4+ | 0 |
| | PU-CVC-1 | Day 3 | 4+ | 0 |
| | | Day 4 | Heavy | Heavy |

Table 81 shows Qualitative efficacy of FTP-M-AgSD-S—CVC-1.

TABLE 81

| | | | Subculture of | Adherence |
|---|---|---|---|---|
| | | | Solution | #colony/cm |
| Inoculum | Group | Day | (around catheter) | catheter |
| E. Coli | FTP-M- | Day 1 | 2+ | 0 |
| (10⁵ CFU/ml) | AgSD- | Day 2 | 4+ | 0 |
| | S--CVC-1 | Day 3 | 4+ | 0 |
| | | Day 4 | 4+ | 0 |
| | | Day 5 | 4+ | 50 |
| | | Day 6 | Heavy | 100 |
| S. Aureus | FTP-M- | Day 1 | 2+ | 0 |
| (10⁵ CFU/ml) | AgSD- | Day 2 | 4+ | 0 |
| | S--CVC-1 | Day 3 | 4+ | 0 |
| | | Day 4 | Heavy | 0 |

TABLE 82
FTP-M-CHX-PU-CVC-Z

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 3 |
| Methanol | 16 |
| Ethylhexyl Glycerin | 2 |
| Silicone Adhesive | 1 |
| Dow Corning MD7-4502 | |
| | 1 |
| Zinc salicylate | 0.2 |
| Tetrahydrofuran | q.s to 100% |

TABLE 83
FTP-M-CHX-PU-CVC-Z-2

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 3 |
| Methanol | 16 |
| Ethylhexyl Glycerin | 2 |
| Silicone Adhesive | 1 |
| Dow Corning MD7-4502 | 1 |
| Zinc salicylate | 0.1 |
| Zinc gluconate | 0.2 |
| Zinc lactate | 0.1 |
| Tetrahydrofuran | q.s to 100% |

TABLE 84
FTP-M-CHX-PU-CVC-Z-3

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Silver sulfadiazine | 1 |
| Methanol | 16 |
| Ethylhexyl Glycerin | 2 |
| Silicone Adhesive | 1 |
| Dow Corning MD7-4502 | 1 |
| Zinc salicylate | 0.2 |
| Tetrahydrofuran | q.s to 100% |

TABLE 85
FTP-M-CHX-PU-CVC-Z-4

| Ingredient | % w/v |
|---|---|
| PA-OCT-80 | 3 |
| Polyurethane | 4 |
| (Tecoflex ® 93A) | |
| Polyurethane | 2 |
| (Tecoflex ® 60D) | |
| Decanediol | 2 |
| Tetrahydrofuran (THF) | 60 |
| Chlorhexidine | 3 |
| Methanol | 16 |
| Ethylhexyl Glycerin | 2 |
| Silicone Adhesive | 1 |
| Loctite M-06FL | 5 |
| Urethane Adhesive | |
| Zinc salicylate | 0.2 |
| Tetrahydrofuran | q.s to 100% |

### Example 22

### Evaluation of Wound Healing Properties of WC Cream Using In-Vivo Rat Model

Method: Excision Wound Model

Excision wounds were used for the study of rate of contraction of wound and epithelization (as discussed in Goswami et al., 2014). Animals were anaesthetized with 80 mg/kg dose of ketamine (i.p.) and the back hairs of the animals were depilated by shaving. An impression was made on the back of neck on the anaesthetized rat. Excision wounds sized 500 mm² and 2 mm depth were made by cutting out layer of skin from the shaven area.

Hemostasis was achieved by blotting the wound with cotton swab soaked in normal saline. The entire wound is left open. The study comprises of four different groups of six animals in each groups as follows and the treatment was done topically with fixed volume of cream in groups:

1. Vehicle control animals: receive injury for wound formation but do not receive any drug treatment but only the placebo cream.

2. WC-AgSD cream: receive injury for wound formation and topical application of WC-AgSD.

3. FTP-AgSD gel: receive injury for wound formation and topical application of FTP-AgSD film forming gel.

4. Standard Silver sulfadiazine cream treated animals (Dr. Reddy Lab SSD cream: receive injury for wound formation and topical application of standard Silver sulfadiazine cream.

At end of study period wound tissues are rapidly removed and stored for histopathological studies.

Measurement of Wound Area:

The progressive changes in wound area were monitored by a camera on predetermined days i.e., 0, 4, 8, 12, 16 and 20. Later on, wound area was measured by using image J software and adobe Photoshop to determine area.

Measurement of Wound Contraction:

Wound contraction was calculated as percentage of the reduction in original wound area size. It was calculated by using the following formula:

$$Percentage wound contraction = \frac{\begin{matrix}Initial area of Wound - \\ {N}^{th} day area of wound\end{matrix}}{Initial area of Wound} \times 100$$

Results:

Table 86 shows percentage reduction in wound size of Rat treated with various silver sulfadiazine formulations.

TABLE 86

| Group | Day 6 (%) | Day 13 (%) |
|---|---|---|
| Placebo | 15 | 60 |
| WC-AgSD | 52 | 85 |
| FTP-AgSD | 53 | 88 |
| Commercial AgSD | 25 | 75 |
| WC-Base | 50 | 82 |

Conclusions:

1. The WC-base (without antimicrobials) show significant wound healing ability when compared to commercial AgSD wound cream in vivo

2. WC-AgSD wound healing formulations show higher wound healing ability in vivo when compared to commercially available AgSD cream.

Although the present technology has been described in relation to particular embodiments thereof, these embodiments and examples are merely exemplary and not intended to be limiting. Many other variations and modifications and other uses will become apparent to those skilled in the art. The present technology should, therefore, not be limited by the specific disclosure herein, and can be embodied in other forms not explicitly described here, without departing from the spirit thereof.

1. A film forming composition comprising: a film forming polymer and an antimicrobial agent; wherein the antimicrobial agent is: a botanical; a silver salt; a zinc salt; polymyxin; chlorhexidine or its salts; benzalkonium chloride; bacitracin; neomycin; clindamycin; polymyxin; bactroban; povidone iodine; gentamicin; gentian violet; mupirocin; dicloxacillin; undecylinic acid; nitrofurazone; miconazole; a cephalosporin; cranberry seed oil; N-acetyl cysteine; berberin; copper sulfate or a combination thereof;
wherein the film forming composition provides controlled release of the antimicrobial agent onto a surface when the film forming composition is contacted with the surface.

2. The film forming composition of claim 1, in the form of a cream comprising:
(a) a zinc salt;
(b) aloe vera gel;
(c) calendula oil or extract; and
(d) rosemary oil.

3. The film forming composition of claim 2, comprising:
(a) 0.1 to 1% zinc oxide;
(b) 0.1 to 0.5% aloe vera gel;
(c) 0.1 to 1% calendula oil or extract; and
(d) 0.05 to 0.5% rosemary oil.

4. The film forming composition of claim 1, wherein the film forming polymer is hydroxyl propyl methyl cellulose stearoxy ether, chitosan or a combination thereof.

5. The film forming composition of claim 1, further comprising 0.1 to 5% flaxseed oil.

6. The film forming composition of claim 1, wherein the film forming polymer is in the form of a triple film forming polymer coating composition (FTP).

7. The film forming composition of claim 6, further comprising a solvent.

8. The film forming composition of claim 7, wherein the solvent is methanol, ethanol or tetrahydrofuran.

9. The film forming composition of claim 6, wherein the FTP comprises: 2 to 20% of a polyacetal-octanediol conjugate (PA-OCT-80); 0.1 to 1% hydroxypropyl methyl cellulose ethoxy ether; 0.1 to 1% chitosan; 0.5 to 20% decanediol; 0.5 to 5% petroleum jelly; 1 to 10% alcohol; and 10 to 70% water.

10. The film forming composition of claim 6, wherein the FTP comprises: 1 to 5% of a polyacetal-octanediol conjugate (PA-OCT-80); 2 to 10% polyurethane; 1 to 5% silicone adhesive; 1 to 3% decanediol; and a solvent; and wherein the film forming composition is chlorhexidine-free.

11. The film forming composition of claim 6, wherein the FTP comprises: 1 to 5% w/w of a polyacetal-octanediol conjugate; 2 to 10% polyurethane; 1 to 5% silicone adhesive; 1 to 3% decanediol; and a solvent.

12. The film forming composition of claim 1, comprising any of the following: 0.01 to 1% silver salt; 0.01 to 0.5% polymyxin; 0.1 to 1.5% bacitracin; 0.1 to 1% neomycin; 5 to 10% povidone iodine; 0.1 to 1% miconazole; 0.1 to 1% undecylinic acid; 0.1 to 1% zinc undecylinate; or 0.05 to 0.5% chlorhexidine.

13. The film forming composition of claim 1, wherein the botanical is an essential oil, an essential oil ingredient, a botanical extract, or a combination thereof.

14. The film forming composition of claim 12, comprising 0.1 to 5% of a botanical extract.

15. The film forming composition of claim 1, wherein the botanical is orange oil, basil oil, tea tree oil, menthol, thymol, grapefruit seed extract, pomegranate extract, red sandalwood, curcumin, witch hazel extract, Vitamin E, Vitamin C, calendula oil or extract, rosemary oil, aloe gel, flaxseed oil or a combination thereof.

16. The film forming composition of claim 4, comprising 0.01 to 0.5% red sandalwood or 0.01 to 0.5% curcumin.

17. The film forming composition of claim 1, wherein the silver salt is: silver sulfadiazine, silver oxide, silver carbonate, silver undecylinate, silversalicylate or a combination thereof.

18. The film forming composition of claim 1, further comprising: ethylhexyl glycerin, benzyl alcohol, caprylic capric triglyceride, glycerin, a combination of behentrimonium methosulfate (and) cetyl alcohol (and) Butylene Glycol (BTMSCB Emulsifier), propanediol or a combination thereof.

19. The film forming composition of claim 1, wherein the composition enhances wound healing by 30% or above within 1 week of application as compared to a placebo treatment.

20. The film forming composition of claim 1, in the form of a coating that increases the infection resistance of a medical device by 1,000 to 10,000 fold when coated on the medical device.

21. The film forming composition of claim 1, wherein the film forming polymer comprises any of Formulas (I)-(VI): 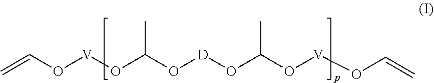
wherein V is 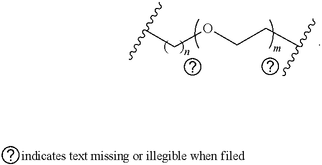
each D may be the same or different and is 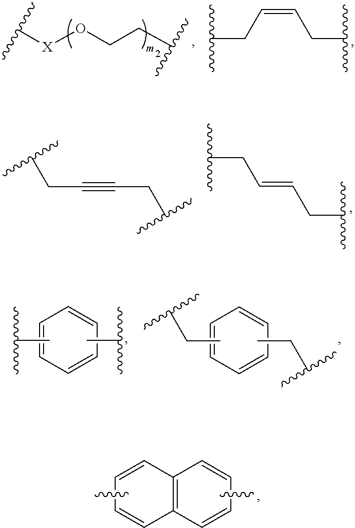
or a therapeutic agent core; each
n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20; and
p is an integer between 3 and 200;
or 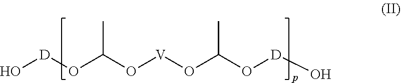
wherein,
V is 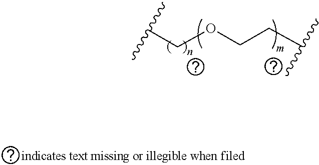
each D may be the same or different and is 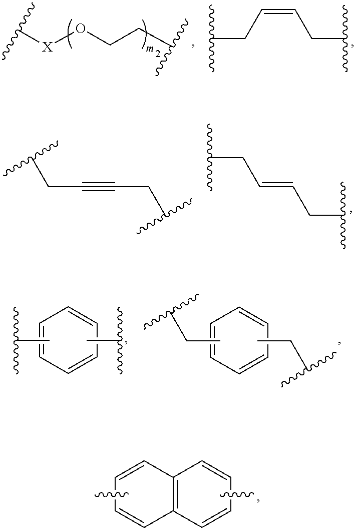
or a therapeutic agent core;
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20; and
p is an integer between 3 and 200;
or 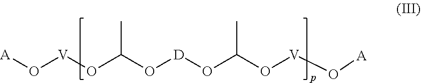
wherein,
A is 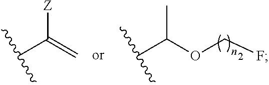
F is 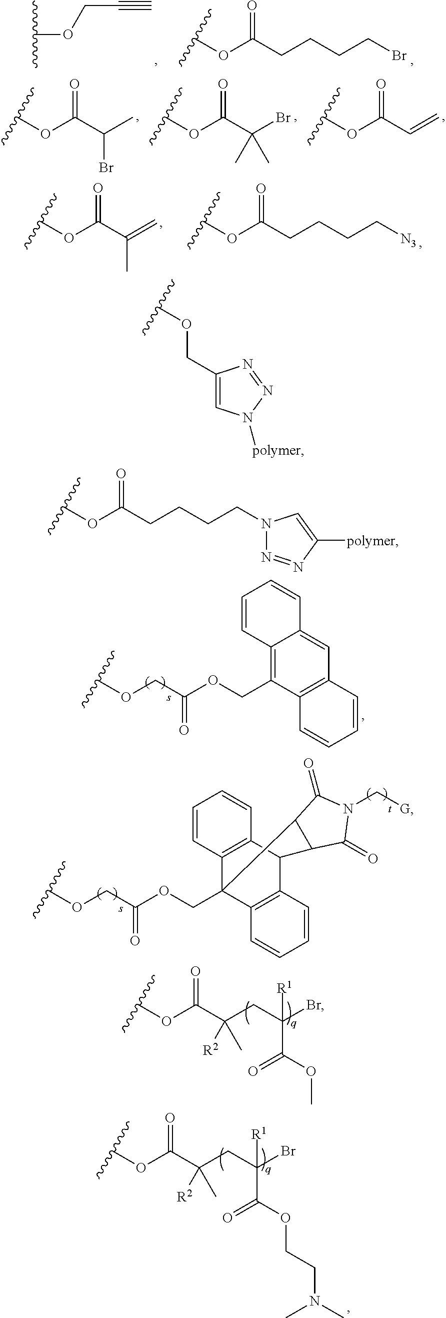 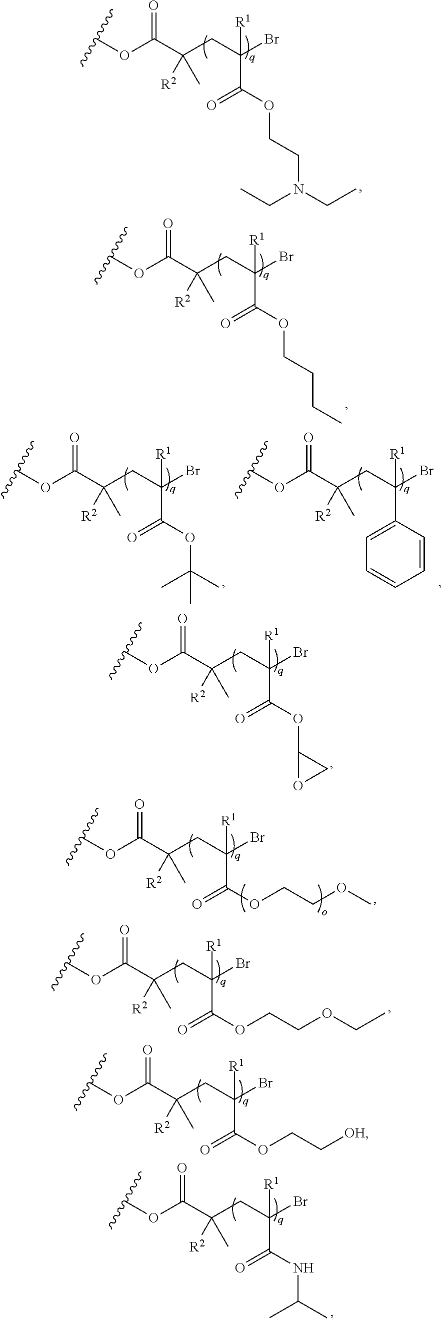
or a polymer;
Z is a polymer, aryl, hetero-aryl, or vinyl;
V is 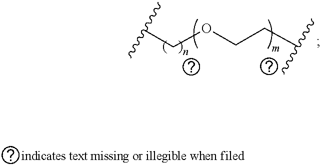
each d may be the same or different and is 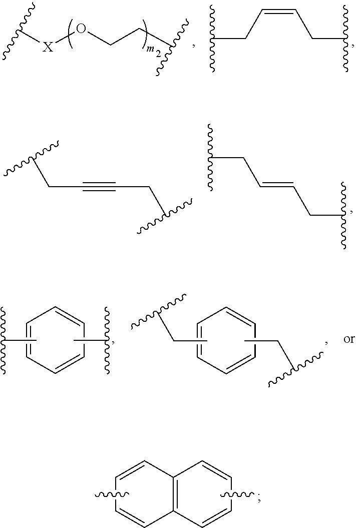
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
n₃ is an integer between 2 and 10;
p is an integer between 3 and 200;
q is an integer between 1 and 100;
s is an integer between 1 and 10;
t is an integer between 1 and 10;
u is an integer between 1 and 100;
G is a polymer, aryl, or alkyl;
R¹ is H or CH₃; and
R² is H or CH₃;
or 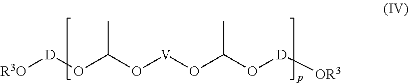
wherein,
R³ is 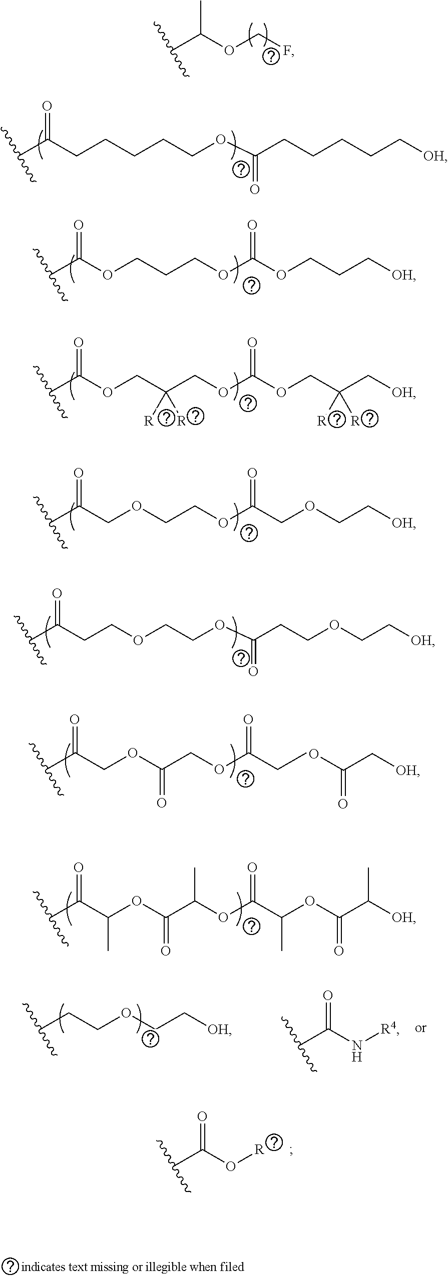
F is 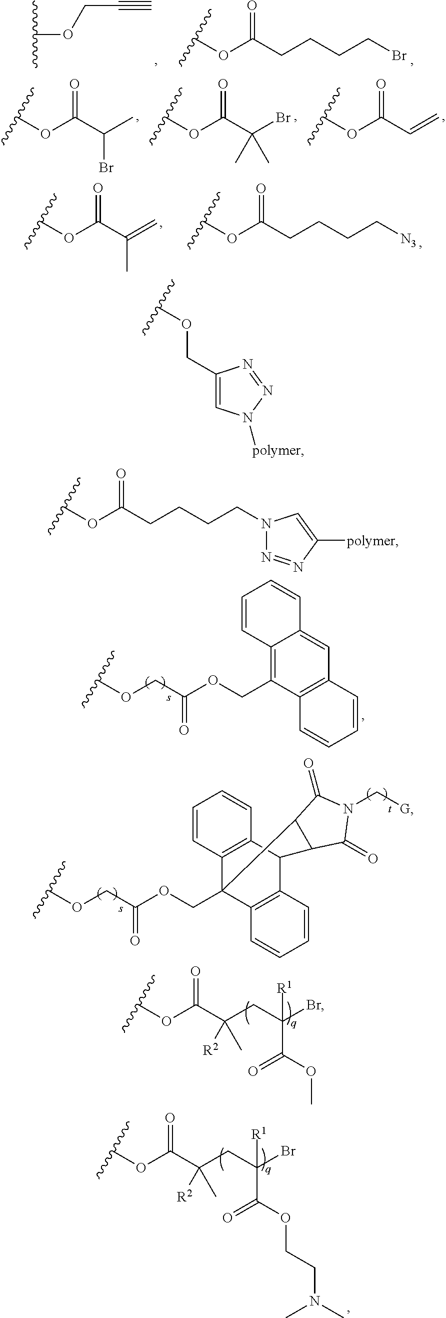 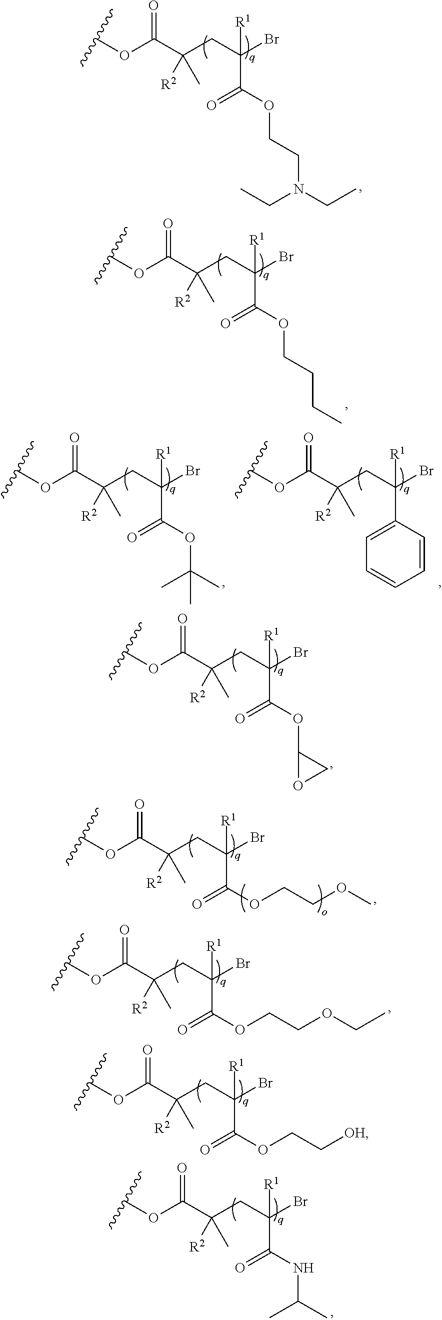
or a polymer;
V is 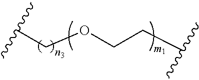
each D may be the same or different and is 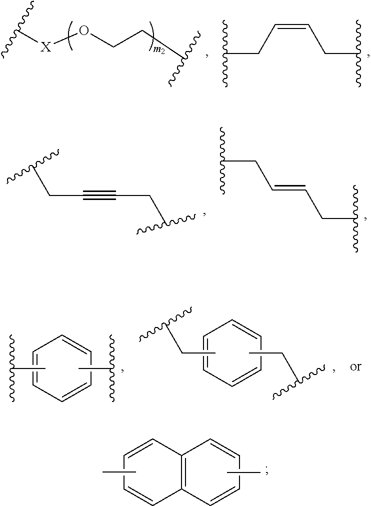
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
n₃ is an integer between 2 and 10;
R¹ is H or CH₃;
R² is H or CH₃;
R⁴ is aryl, alkyl, or a polymer;
R⁵ is aryl, alkyl, or a polymer;
R⁷ is H or halogen;
p is an integer between 3 and 200;
q is an integer between 1 and 100;
r is an integer between 0 and 100;
s is an integer between 1 and 10;
t is an integer between 1 and 10;
u is an integer between 1 and 100; and
G is a polymer, aryl, or alkyl;
or 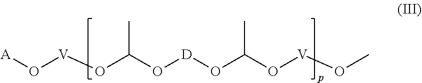
wherein
A is 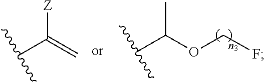
F is 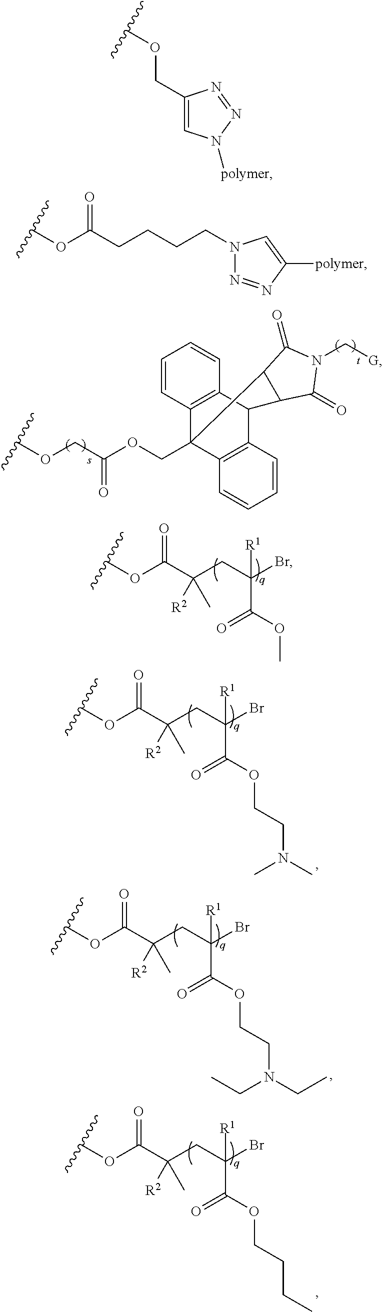 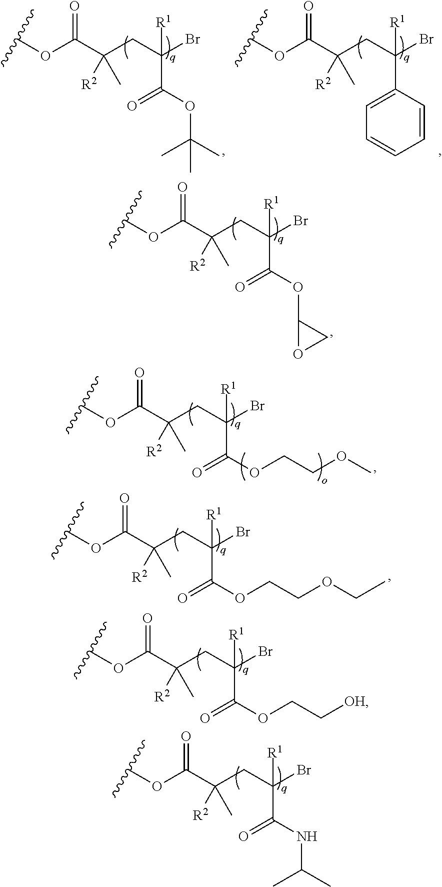
or a polymer;
V is 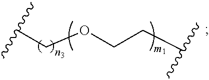
each D may be the same or different and is 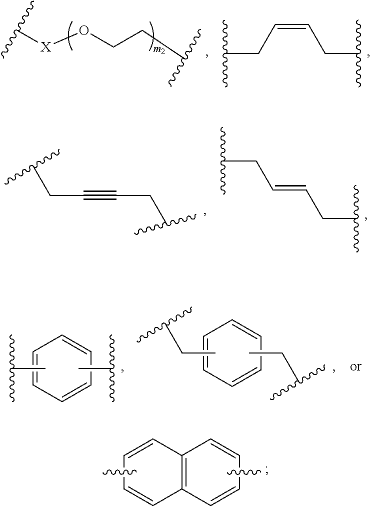
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20; n₃ is an integer between 2 and 10;
G is a polymer;
Z is a polymer;
R¹ is H or CH₃;
R² is H or CH₃;
p is an integer between 3 and 200;
q is an integer between 1 and 100;
s is an integer between 1 and 10;
t is an integer between 1 and 10;
u is an integer between 1 and 100;
or, 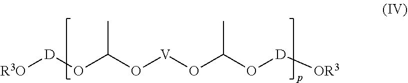
wherein,
R³ is 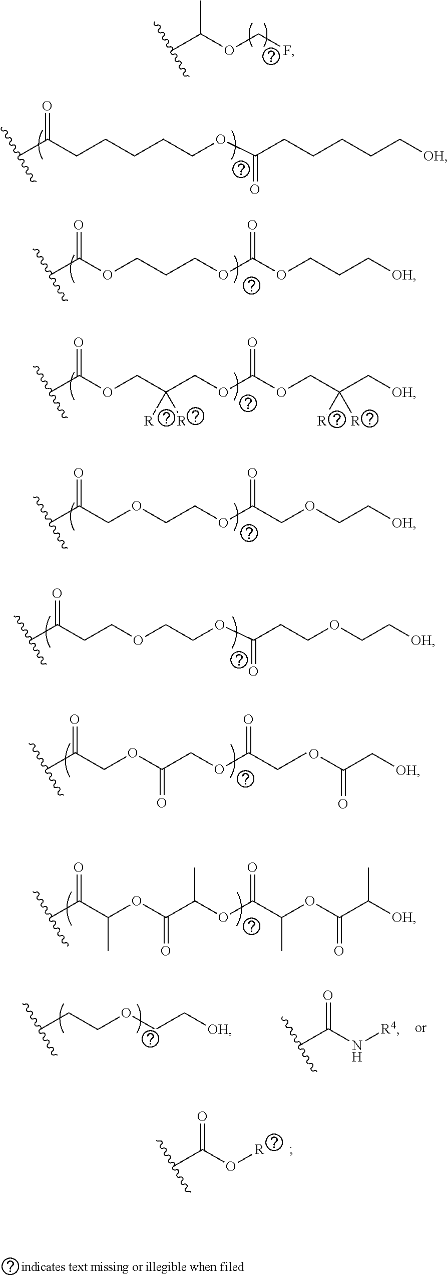
F is 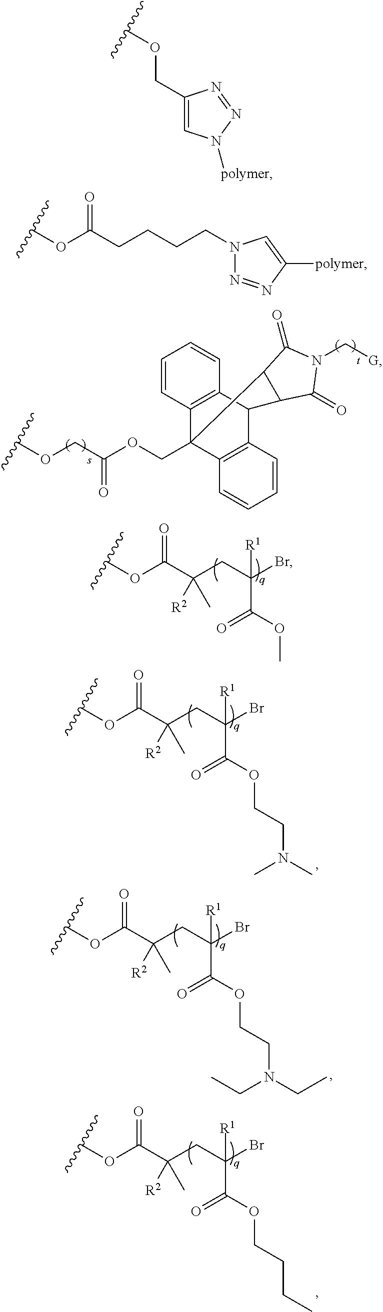 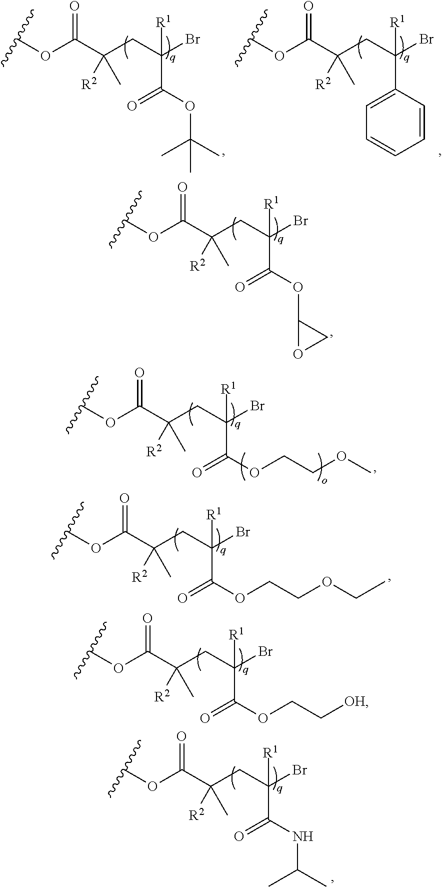
or a polymer;
V is 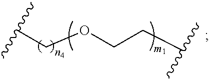
each D may be the same or different and is 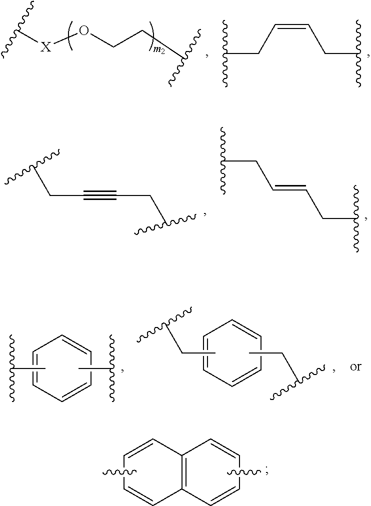
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20; n₃ is an integer between 2 and 10;
G is a polymer;
R¹ is H or CH₃;
R² is H or CH₃;
R⁴ is a polymer;
R⁵ is a polymer;
R⁷ is H or halogen;
p is an integer between 3 and 200;
q is an integer between 1 and 100;
r is an integer between 0 and 100;
s is an integer between 1 and 10;
t is an integer between 1 and 10; and
u is an integer between 1 and 100;
or 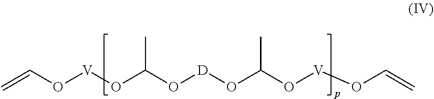
wherein,
V is 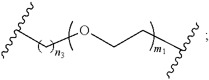
each D may be the same or different and is 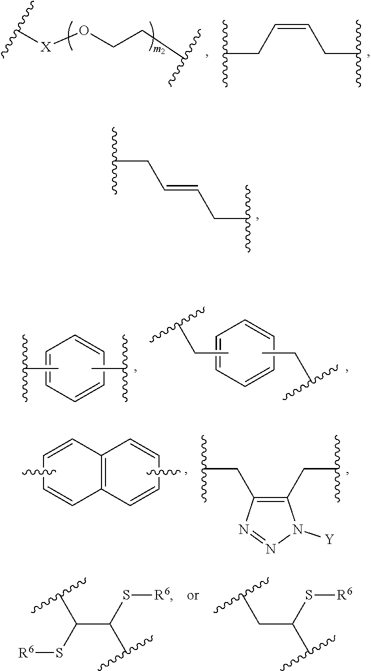
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
p is an integer between 3 and 200;
Y is a polymer or therapeutic agent; and
R⁶ is alkyl, aryl, or a polymer,
or 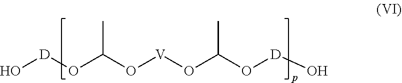
wherein,
V is 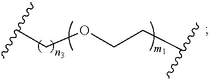
each D may be the same or different and is 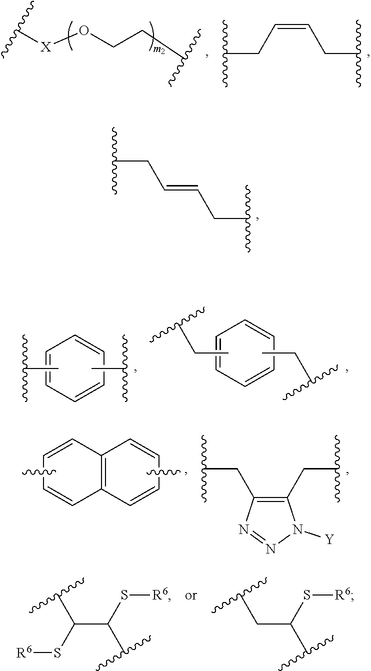
each n₁ may be the same or different and is an integer between 2 and 10;
each m₁ may be the same or different and is an integer between 0 and 20;
each X may be the same or different and is C₂-C₁₀ alkyl;
each m₂ may be the same or different and is an integer between 0 and 20;
p is an integer between 3 and 200;
Y is a polymer or therapeutic agent; and
R⁶ is alkyl, aryl, or a polymer.

22. A film forming composition comprising a mixture of:
(a) a pH-degradable polyacetal co-polymer or polyacetal-octanediol conjugate, or polyketal co-polymer or polyketal-octanediol conjugate;
(b) a hydrophilic polymer; and
(c) a hydrophobic-hydrophilic polymer.

23. A method of treating a wound comprising contacting a composition of claim 1 with the wound.

24. A chlorhexidine-free coating composition that increases the infection resistance of a medical device when coated on the medical device, the coating composition comprising: a triple film forming polymer coating composition (FTP) comprising polyacetal-octanediol conjugate (PA-OCT or PA-OCT-80); a first polyurethane composition; a second polyurethane composition; a silicone adhesive; decanediol; and a solvent wherein the solvent is methanol, ethanol or tetrahydrofuran.

25. The coating composition of claim 24 comprising 1 to 5% of the first polyurethane composition; 1 to 5% of the second polyurethane composition; 1 to 5% silicone adhesive; and 1 to 3% decanediol.

26. The coating composition of claim 24, wherein the solvent comprises 5 to 30% methanol; 5 to 30% ethanol; or 50 to 70% tetrahydrofuran.

27. The coating composition of claim 24, wherein the solvent comprises 20 to 80% methanol; 20 to 80% methanol; or 5 to 20% tetrahydrofuran.

28. The coating composition of claim 24, further comprising: a silver salt, a zinc salt, ethyl hexyl glycerin, mandelic acid, a urethane adhesive, lactic acid, N-acetyl cysteine or a combination thereof.

29. The coating composition of claim 28, wherein the silver salt is silver sulfadiazine, silver carbonate, silver oxide, silver nitrate or a combination thereof.

30. The coating composition of claim 28, comprising 0.1 to 1% silver salt; or 1 to 5% ethyl hexyl glycerin; or 0.5 to 3% mandelic acid; or 1 to 15% urethane adhesive; or 0.5 to 2% lactic acid.

31. The coating composition of claim 28, wherein the zinc salt is zinc gluconate, zinc lactate, zinc salicylate, zinc acetate, zinc citrate or a combination thereof.

32. The coating composition of claim 24, comprising 0.1 to 2% zinc salt.

33. The coating composition of claim 24, comprising 1 to 5% of the FTP.

34. A medical device coated with the coating composition of claim 24.

35. A medical or veterinary composition comprising the composition of claim 1.

36. A coating composition that increases the infection resistance of a medical device when coated on the medical device, the coating composition comprising:
(a) 1 to 5% chlorhexidine;
(b) 0.1 to 1% of a zinc salt;
(c) 0.2 to 5% of a triple film forming polymer coating composition (FTP) comprising polyacetal-octanediol conjugate (PA-OCT or PA-OCT-80);
(d) 0.2 to 5% of a first polyurethane composition;
(e) 0.2 to 5% of a second polyurethane composition;
(f) 0.2 to 5% of a silicone adhesive;
(g) 0.5 to 3% decanediol; and
(h) a solvent, wherein the solvent is methanol, ethanol or tetrahydrofuran.

37. The medical device of claim 34, wherein the medical device is a urinary catheter, a central venous catheter, a peritoneal dialysis catheter, an endotracheal tube, a hernia patch or a wound dressing.

38. The medical device of claim 34, wherein the medical device is a biomedical polymer comprising polyurethane, silicone, polyvinyl chloride (PVC), polytetrafluoroethylene (PTFE) or cotton.

39. A method of rendering the inner lumen of a medical device biofilm resistant, the method comprising; contacting the inner lumen with a composition of claim 1.

40. The method of claim 39, wherein the inner surface of the medical device is contacted with the composition for 5 to 60 seconds, and then removed from contact and dried for 24 to 48 hours.

41. The method of claim 39, wherein the biofilm resistance of the inner lumen of the medical device is 1,000 to 10,000 fold more than the biofilm resistance of the inner lumen of a medical device that has not been contacted with the composition.

42. Use of a composition of claim 1 for treatment of a human.

43. Use of a composition of claim 1 for treatment of an animal.
